SEQUENCE LISTING (470 sequences)

SEQ ID NO: 1 - protein, 4 aa, synthetic construct
GGGG

SEQ ID NO: 2 - protein, 5 aa, synthetic construct
GGGGG

SEQ ID NO: 3 - protein, 6 aa, synthetic construct
GGGGGG

SEQ ID NO: 4 - protein, 7 aa, synthetic construct
GGGGGGG

SEQ ID NO: 5 - protein, 8 aa, synthetic construct
GGGGGGGG

SEQ ID NO: 6 - protein, 5 aa, synthetic construct
  VARIANT 1..5: note = The entire sequence of amino acids 1-5 can be repeated one or more times
GGGGS

SEQ ID NO: 7 - protein, 10 aa, synthetic construct
GGGGSGGGGS

SEQ ID NO: 8 - protein, 6 aa, synthetic construct
GGGGGS

SEQ ID NO: 9 - protein, 4 aa, synthetic construct
KERE

SEQ ID NO: 10 - protein, 4 aa, synthetic construct
KQRE

SEQ ID NO: 11 - protein, 4 aa, synthetic construct
GLEW

SEQ ID NO: 12 - protein, 5 aa, synthetic construct
KEREL

SEQ ID NO: 13 - protein, 5 aa, synthetic construct
KEREF

SEQ ID NO: 14 - protein, 5 aa, synthetic construct
KQREL

SEQ ID NO: 15 - protein, 5 aa, synthetic construct
KQREF

SEQ ID NO: 16 - protein, 5 aa, synthetic construct
KEREG

SEQ ID NO: 17 - protein, 5 aa, synthetic construct
KQREW

SEQ ID NO: 18 - protein, 5 aa, synthetic construct
KQREG

SEQ ID NO: 19 - protein, 4 aa, synthetic construct
TERE

SEQ ID NO: 20 - protein, 5 aa, synthetic construct
TEREL

SEQ ID NO: 21 - protein, 4 aa, synthetic construct
TQRE

SEQ ID NO: 22 - protein, 5 aa, synthetic construct
TQREL

SEQ ID NO: 23 - protein, 4 aa, synthetic construct
KECE

SEQ ID NO: 24 - protein, 5 aa, synthetic construct
KECEL

SEQ ID NO: 25 - protein, 5 aa, synthetic construct
KECER

SEQ ID NO: 26 - protein, 4 aa, synthetic construct
KQCE

SEQ ID NO: 27 - protein, 5 aa, synthetic construct
KQCEL

SEQ ID NO: 28 - protein, 4 aa, synthetic construct
RERE

SEQ ID NO: 29 - protein, 5 aa, synthetic construct
REREG

SEQ ID NO: 30 - protein, 4 aa, synthetic construct
RQRE

SEQ ID NO: 31 - protein, 5 aa, synthetic construct
RQREL

SEQ ID NO: 32 - protein, 5 aa, synthetic construct
RQREF

SEQ ID NO: 33 - protein, 5 aa, synthetic construct
RQREW

SEQ ID NO: 34 - protein, 4 aa, synthetic construct
QERE

SEQ ID NO: 35 - protein, 5 aa, synthetic construct
QEREG

SEQ ID NO: 36 - protein, 4 aa, synthetic construct
QQRE

SEQ ID NO: 37 - protein, 5 aa, synthetic construct
QQREW

SEQ ID NO: 38 - protein, 5 aa, synthetic construct
QQREL

SEQ ID NO: 39 - protein, 5 aa, synthetic construct
QQREF

SEQ ID NO: 40 - protein, 4 aa, synthetic construct
KGRE

SEQ ID NO: 41 - protein, 5 aa, synthetic construct
KGREG

SEQ ID NO: 42 - protein, 4 aa, synthetic construct
KDRE

SEQ ID NO: 43 - protein, 5 aa, synthetic construct
KDREV

SEQ ID NO: 44 - protein, 5 aa, synthetic construct
DECKL

SEQ ID NO: 45 - protein, 5 aa, synthetic construct
NVCEL

SEQ ID NO: 46 - protein, 4 aa, synthetic construct
GVEW

SEQ ID NO: 47 - protein, 4 aa, synthetic construct
EPEW

SEQ ID NO: 48 - protein, 4 aa, synthetic construct
GLER

SEQ ID NO: 49 - protein, 4 aa, synthetic construct
DQEW

SEQ ID NO: 50 - protein, 4 aa, synthetic construct
DLEW

SEQ ID NO: 51 - protein, 4 aa, synthetic construct
GIEW

SEQ ID NO: 52 - protein, 4 aa, synthetic construct
ELEW

SEQ ID NO: 53 - protein, 4 aa, synthetic construct
GPEW

SEQ ID NO: 54 - protein, 4 aa, synthetic construct
EWLP

SEQ ID NO: 55 - protein, 4 aa, synthetic construct
GPER

SEQ ID NO: 56 - protein, 5 aa, synthetic construct
  VARIANT 1..5: note = The entire sequence of amino acids 1-5 can be repeated one to ten times
GGGGS

SEQ ID NO: 57 - protein, 15 aa, synthetic construct
GGGGSGGGGSGGGGS

SEQ ID NO: 58 - protein, 20 aa, synthetic construct
GGGGSGGGGSGGGGSGGGGS

SEQ ID NO: 59 - protein, 399 aa, synthetic construct
DVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGSEVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 60 - protein, 685 aa, synthetic construct
DVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGSEVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGLVQAGGSLRLSCVASGRTFSATNMGWFRQAPGKEREFVGIISESGRNTDYADSVKGRFTITRDNAKSTVYLQMNNLKPEDTAVYYCALGRDYFATDTRQYNYWGLGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 61 - protein, 683 aa, synthetic construct
DVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGSEVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGGSGGGGSGGGGSEVQLVESGGGWVQPGGSLRLSCSPSERLSSIDIMGWYRQTPGKEREWVATITGDDTTHYENSVKGRFTISRDTAKNMVYLQMNSLEPEDTAVYYCNAGILPREEYWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 62 - protein, 681 aa, synthetic construct
DVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGSEVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGSEVQLVESGGGLVQAGTSLRLSCAASGTFFRINDMGWYRQAPGRQRELVAYITSGGSTHYADSLKGRFTISRDNAKNTILLQMNSLKPEDTGVYYCYTDIEARLNEWRTVWGQGTLVTVSSGGGGSGGGGSGGGGSGGGGSEVQLVESGGGSVQPGGSLRLSCTASGLTLDYYAIGWFRQAPGKEREGVSCISSSSSDGSTLYADSVKGRFTISRDNDKNTVYLQLNSLKPEDTGVYYCAADILNGECYSDYFEPLSGYDFDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 63 - protein, 655 aa, synthetic construct
DVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGSEVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGSEVQLVESGGGLVQAGGALRLSCAVSRNIFSNDVIAWYRQAPGKQREWIAQVSTDGSTQYAHSVKGRFTISRDSAGNTVYLRMDSLKPEDTAVYLCNTYLPSIPWGQGTLVTVSSGGGGSGGGSEVQLVESGGGLVQAGGSLSLSCLGSGPTFSEYPIGWFRQAPGMEREFVAAISGSGGRPYFADSVKGRFSVSRDNAKNTVYLQMNSLKPEDTAVYYCAALNYYSSAGTHSTPAYWGQGTQVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 64 - skipped

SEQ ID NO: 65 - protein, 7 aa, synthetic construct
SGGSGGS

SEQ ID NO: 66 - protein, 8 aa, synthetic construct
GGGGSGGS

SEQ ID NO: 67 - protein, 9 aa, synthetic construct
GGGGSGGGS

SEQ ID NO: 68 - protein, 18 aa, synthetic construct
GGGGSGGGGSGGGGSGGS

SEQ ID NO: 69 - protein, 25 aa, synthetic construct
GGGGSGGGGSGGGGSGGGGSGGGGS

SEQ ID NO: 70 - protein, 30 aa, synthetic construct
GGGGSGGGGSGGGGSGGGGSGGGGSGGGGS

SEQ ID NO: 71 - protein, 35 aa, synthetic construct
GGGGSGGGGSGGGGSGGGGSGGGGSGGGGSGGGGS

SEQ ID NO: 72 - protein, 40 aa, synthetic construct
GGGGSGGGGSGGGGSGGGGSGGGGSGGGGSGGGGSGGGGS

SEQ ID NO: 73 - protein, 15 aa, synthetic construct
EPKSCDKTHTCPPCP

SEQ ID NO: 74 - protein, 24 aa, synthetic construct
GGGGSGGGSEPKSCDKTHTCPPCP

SEQ ID NO: 75 - protein, 12 aa, synthetic construct
EPKTPKPQPAAA

SEQ ID NO: 76 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 77 - protein, 115 aa, synthetic construct [sequence omitted]

SEQ ID NO: 78 - protein, 115 aa, synthetic construct [sequence omitted]

SEQ ID NO: 79 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 80 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 81 - protein, 115 aa, synthetic construct [sequence omitted]

SEQ ID NO: 82 - protein, 116 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 83 - protein, 115 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 84 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 85 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 86 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 87 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 88 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 89 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 90 - protein, 115 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 91 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 92-96 - skipped (5 entries)

SEQ ID NO: 97 - protein, 25 aa, synthetic construct
EVQLVESGGGLVQAGGSLRLSCVAS

SEQ ID NO: 98-103 - skipped (6 entries)

SEQ ID NO: 104 - protein, 25 aa, synthetic construct
EVQLVESGGGLVQAGDSTTISCVAS

SEQ ID NO: 105-113 - skipped (9 entries)

SEQ ID NO: 114 - protein, 10 aa, synthetic construct
GRTFSATNMG

SEQ ID NO: 115-120 - skipped (6 entries)

SEQ ID NO: 121 - protein, 10 aa, synthetic construct
GGSFATYAFG

SEQ ID NO: 122-130 - skipped (9 entries)

SEQ ID NO: 131 - protein, 14 aa, synthetic construct
WFRQAPGKEREFVG

SEQ ID NO: 132-137 - skipped (6 entries)

SEQ ID NO: 138 - protein, 14 aa, synthetic construct
WFRQAPGKEREFVA

SEQ ID NO: 139-147 - skipped (9 entries)

SEQ ID NO: 148 - protein, 10 aa, synthetic construct
IISESGRNTD

SEQ ID NO: 149-154 - skipped (6 entries)

SEQ ID NO: 155 - protein, 10 aa, synthetic construct
TISQRGSTSY

SEQ ID NO: 156-164 - skipped (9 entries)

SEQ ID NO: 165 - protein, 39 aa, synthetic construct
YADSVKGRFTITRDNAKSTVYLQMNNLKPEDTAVYYCAL

SEQ ID NO: 166-171 - skipped (6 entries)

SEQ ID NO: 172 - protein, 39 aa, synthetic construct
YSDSVEGRFTISKDNAKSTVYLQMNSLQPEDTAVYYCAA

SEQ ID NO: 173-181 - skipped (9 entries)

SEQ ID NO: 182 - protein, 14 aa, synthetic construct
GRDYFATDTRQYNY

SEQ ID NO: 183-188 - skipped (6 entries)

SEQ ID NO: 189 - protein, 14 aa, synthetic construct
RYYGVDYRSTSYDF

SEQ ID NO: 190-329 - skipped (140 entries)

SEQ ID NO: 330 - protein, 25 aa, synthetic construct
EVQLVESGGGWVQPGGSLRLSCSPS

SEQ ID NO: 331 - protein, 10 aa, synthetic construct
ERLSSIDIMG

SEQ ID NO: 332 - protein, 14 aa, synthetic construct
WYRQTPGKEREWVA

SEQ ID NO: 333 - skipped

SEQ ID NO: 334 - protein, 9 aa, synthetic construct
TITGDDTTH

SEQ ID NO: 335 - protein, 39 aa, synthetic construct
YENSVKGRFTISRDTAKNMVYLQMNSLEPEDTAVYYCNA

SEQ ID NO: 336 - protein, 8 aa, synthetic construct
GILPREEY

SEQ ID NO: 337 - protein, 25 aa, synthetic construct
EVQLVESGGGLVQAGGSLRLSCAAS

SEQ ID NO: 338 - protein, 10 aa, synthetic construct
GRSFSSYYMG

SEQ ID NO: 339 - protein, 14 aa, synthetic construct
WFRQAPGKEREFVA

SEQ ID NO: 340 - protein, 10 aa, synthetic construct
GINWSGRSTH

SEQ ID NO: 341 - protein, 39 aa, synthetic construct
YADSVKGRFTISRDNAKNTVYLQMNSLKPEDRAAYYCAA

SEQ ID NO: 342 - protein, 19 aa, synthetic construct
DSAPSTRIGVRSLWDDYDY

SEQ ID NO: 343-346 - skipped (4 entries)

SEQ ID NO: 347 - protein, 123 aa, synthetic construct
EVQLVESGGGLVQAGGSLRLSCVASGRTFSATNMGWFRQAPGKEREFVGIISESGRNTDYADSVKGRFTITRDNAKSTVYLQMNNLKPEDTAVYYCALGRDYFATDTRQYNYWGLGTLVTVSS

SEQ ID NO: 348-353 - skipped (6 entries)

SEQ ID NO: 354 - protein, 123 aa, synthetic construct
EVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSS

SEQ ID NO: 355-358 - skipped (4 entries)

SEQ ID NO: 359 - protein, 116 aa, synthetic construct
EVQLVESGGGWVQPGGSLRLSCSPSERLSSIDIMGWYRQTPGKEREWVATITGDDTTHYENSVKGRFTISRDTAKNMVYLQMNSLEPEDTAVYYCNAGILPREEYWGQGTLVTVSS

SEQ ID NO: 360 - protein, 128 aa, synthetic construct
EVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSS

SEQ ID NO: 361 - protein, 127 aa, synthetic construct
EVQLVESGGGLVQSGGSLRLSCAATGFTLDYYVIGWFRQAPGKEREGVACLSSNGDRINYADSVKGRFTISRDIAKNTVYLQMNSLKPEDTAVYHCAAGTSTTVRDMCGIMYLYDYWAQGTLVTVSS

SEQ ID NO: 362 - protein, 127 aa, synthetic construct
DVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSS

SEQ ID NO: 363 - protein, 127 aa, synthetic construct
EVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSS

SEQ ID NO: 364 - protein, 685 aa, synthetic construct
DVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQAGGSLRLSCVASGRTFSATNMGWFRQAPGKEREFVGIISESGRNTDYADSVKGRFTITRDNAKSTVYLQMNNLKPEDTAVYYCALGRDYFATDTRQYNYWGLGTLVTVSSGGGGSGGGGSGGGGSGGGGSEVQLVESGGGWVQPGGSLRLSCSPSERLSSIDIMGWYRQTPGKEREWVATITGDDTTHYENSVKGRFTISRDTAKNMVYLQMNSLEPEDTAVYYCNAGILPREEYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 365 - protein, 685 aa, synthetic construct
DVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGWVQPGGSLRLSCSPSERLSSIDIMGWYRQTPGKEREWVATITGDDTTHYENSVKGRFTISRDTAKNMVYLQMNSLEPEDTAVYYCNAGILPREEYWGQGTLVTVSSGGGGSGGGGSGGGGSGGGGSEVQLVESGGGLVQAGGSLRLSCVASGRTFSATNMGWFRQAPGKEREFVGIISESGRNTDYADSVKGRFTITRDNAKSTVYLQMNNLKPEDTAVYYCALGRDYFATDTRQYNYWGLGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 366 - protein, 402 aa, synthetic construct
DVQLVESGGGLVQAGGSLRLSCVASGRTFSATNMGWFRQAPGKEREFVGIISESGRNTDYADSVKGRFTITRDNAKSTVYLQMNNLKPEDTAVYYCALGRDYFATDTRQYNYWGLGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 367 - protein, 692 aa, synthetic construct
DVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQAGGSLRLSCVASGRTFSATNMGWFRQAPGKEREFVGIISESGRNTDYADSVKGRFTITRDNAKSTVYLQMNNLKPEDTAVYYCALGRDYFATDTRQYNYWGLGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 368 - protein, 402 aa, synthetic construct
DVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGLVQAGGSLRLSCVASGRTFSATNMGWFRQAPGKEREFVGIISESGRNTDYADSVKGRFTITRDNAKSTVYLQMNNLKPEDTAVYYCALGRDYFATDTRQYNYWGLGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 369 - protein, 692 aa, synthetic construct
DVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGLVQAGGSLRLSCVASGRTFSATNMGWFRQAPGKEREFVGIISESGRNTDYADSVKGRFTITRDNAKSTVYLQMNNLKPEDTAVYYCALGRDYFATDTRQYNYWGLGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 370 - protein, 685 aa, synthetic construct; identical to SEQ ID NO: 60

SEQ ID NO: 371 - protein, 690 aa, synthetic construct
DVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGWVQPGGSLRLSCSPSERLSSIDIMGWYRQTPGKEREWVATITGDDTTHYENSVKGRFTISRDTAKNMVYLQMNSLEPEDTAVYYCNAGILPREEYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 372 - protein, 400 aa, synthetic construct
DVQLVESGGGWVQPGGSLRLSCSPSERLSSIDIMGWYRQTPGKEREWVATITGDDTTHYENSVKGRFTISRDTAKNMVYLQMNSLEPEDTAVYYCNAGILPREEYWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 373 - protein, 690 aa, synthetic construct
DVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGWVQPGGSLRLSCSPSERLSSIDIMGWYRQTPGKEREWVATITGDDTTHYENSVKGRFTISRDTAKNMVYLQMNSLEPEDTAVYYCNAGILPREEYWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 374 - protein, 683 aa, synthetic construct; identical to SEQ ID NO: 61

SEQ ID NO: 375 - protein, 679 aa, synthetic construct
DVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGWVQPGGSLRLSCSPSERLSSIDIMGWYRQTPGKEREWVATITGDDTTHYENSVKGRFTISRDTAKNMVYLQMNSLEPEDTAVYYCNAGILPREEYWGQGTLVTVSSGGGGSGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 376 - protein, 675 aa, synthetic construct
DVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGWVQPGGSLRLSCSPSERLSSIDIMGWYRQTPGKEREWVATITGDDTTHYENSVKGRFTISRDTAKNMVYLQMNSLEPEDTAVYYCNAGILPREEYWGQGTLVTVSSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 377 - protein, 407 aa, synthetic construct
DVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 378 - protein, 697 aa, synthetic construct
DVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 379 - protein, 690 aa, synthetic construct
DVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGSEVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 380 - protein, 686 aa, synthetic construct
DVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 381 - protein, 682 aa, synthetic construct
DVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 382 - protein, 407 aa, synthetic construct
DVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 383 - protein, 697 aa, synthetic construct
DVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQPGGSLRLSCAASGRTFSGNTMGWFRQAPGKEREFVAAISSSGRNTNYADPVEVRFTISRDNAKNTVYLQMNSLQPEDTAVYYCAASSWAAAAGTIANIYDYWGQGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 384 - protein, 690 aa, synthetic construct
DVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGSEVQLVESGGGVVQPGGSLRLSCAATGFTLDYYVIGWFRQAPGKEREGVACLSSNGDRINYADSVKGRFTISRDIAKNTVYLQMNSLRPEDTALYHCAAGTSTTVRDMCGIMYLYDYWAQGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGGSGGGGSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 385 - protein, 686 aa, synthetic construct
DVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 386 - protein, 682 aa, synthetic construct
DVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGVVQPGGSLRLSCAASGRTFSGNTMGWFRQAPGKEREFVAAISSTGRNTNYADSVEGRFTISRDNSKNTVYLQMNSLRPEDTALYYCAASSWAAAAGTIANIYDYWGQGTLVTVSSGGGGSGGGGSGGGGSGGGGSEVQLVESGGGLVQAGDSTTISCVASGGSFATYAFGWFRQAPGKEREFVATISQRGSTSYYSDSVEGRFTISKDNAKSTVYLQMNSLQPEDTAVYYCAARYYGVDYRSTSYDFWGQGTLVTVSSGGGGSEVQLVESGGGLVQAGGSLRLSCAASGRSFSSYYMGWFRQAPGKEREFVAGINWSGRSTHYADSVKGRFTISRDNAKNTVYLQMNSLKPEDRAAYYCAADSAPSTRIGVRSLWDDYDYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 387-389 - skipped (3 entries)

SEQ ID NO: 390 - protein, 551 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 391 - protein, 369 aa, synthetic construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 392-418 - skipped (27 entries)

SEQ ID NO: 419 - protein, 159 aa, synthetic construct
MYRMQLLSCIALSLALVTNSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTHHHHHH

SEQ ID NO: 420 - protein, 153 aa, synthetic construct
MYRMQLLSCIALSLALVTNSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 421 - protein, 399 aa, synthetic construct; identical to SEQ ID NO: 59

SEQ ID NO: 422 - protein, 685 aa, synthetic construct; identical to SEQ ID NO: 60

SEQ ID NO: 423 - protein, 683 aa, synthetic construct; identical to SEQ ID NO: 61

SEQ ID NO: 424 - protein, 115 aa, synthetic construct
QVQLVESGGGLVQPGGSLRLSCAASGFTFSDYWMYWVRQAPGKGLEWVSEINTNGLITKYPDSVKGRFTISRDNAKNTLYLQMNSLKPEDTALYYCARSPSGFNRGQGTQVTVSS

SEQ ID NO: 425 - protein, 129 aa, synthetic construct
AVQLVESGGGLVQPGDSLRLSCAASGRTFSDYSGYTYTVGWFRQAPGKEREFVARIYWSSGNTYYADSVKGRFTISRDIAKNTVDLLMNNLEPEDTAVYYCAARDGIPTSRSVESYNYWGQGTQVTVSS

SEQ ID NO: 426 - protein, 127 aa, synthetic construct
AVQLVDSGGGLVQAGGSLRLSCAASGRTFSAHSVYTMGWFRQAPGKEREFVARIYWSSANTYYADSVKGRFTISRDNAKNTVDLLMNCLKPEDTAVYYCAARDGIPTSRSVEAYNYWGQGTQVTVSS

SEQ ID NO: 427 - protein, 123 aa, synthetic construct
QVQLVESGGGLVQAGGSLSLSCAASGRSFTGYYMGWFRQAPGKERQLLASISWRGDNTYYKESVKGRFTISRDDAKNTIYLQMNSLKPEDTAVYYCAASILPLSDDPGWNTNWGQGTQVTVSS

SEQ ID NO: 428 - protein, 123 aa, synthetic construct
EVQLVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVSS

SEQ ID NO: 429 - protein, 117 aa, synthetic construct
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 430 - protein, 116 aa, synthetic construct
EVQLVESGGGLVQEGGSLRLACAASERIWDINLLGWYRQGPGNERELVATITVGDSTSYADSVKGRFTISRDYDKNTLYLQMNSLRPEDTGLYYCKIRRTWHSELWGQGTQVTVSS

SEQ ID NO: 431 - protein, 115 aa, synthetic construct [sequence omitted]

SEQ ID NO: 432 - protein, 116 aa, synthetic construct
AVQLVDSGGGLVQPGGSLRLSCAASGFSFGSFGMSWVRQYPGKEPEWVSSINGRGDDTRYADSVKGRFSISRDNAKNTLYLQMNSLKPEDTAEYYCTIGRSVSRSRTQGTQVTVSS

SEQ ID NO: 433 - protein, 115 aa, synthetic construct [sequence omitted]

SEQ ID NO: 434 - protein, 5 aa, synthetic construct
VTVSS

SEQ ID NO: 435 - protein, 11 aa, synthetic construct
WGQGTLVTVSS

SEQ ID NO: 436 - protein, 11 aa, synthetic construct
WGLGTLVTVSS

SEQ ID NO: 437 - protein, 11 aa, synthetic construct
WGQGTLVTVSS

SEQ ID NO: 438 - protein, 11 aa, synthetic construct
WGQGTLVTVSS

SEQ ID NO: 439 - protein, 30 aa, synthetic construct
EVQLVESGGGLVQAGDSTTISCVASGGSFA

SEQ ID NO: 440 - protein, 30 aa, synthetic construct
EVQLVESGGGLVQAGGSLRLSCVASGRTFS

SEQ ID NO: 441 - protein, 30 aa, synthetic construct
EVQLVESGGGWVQPGGSLRLSCSPSERLSS

SEQ ID NO: 442 - protein, 30 aa, synthetic construct
EVQLVESGGGLVQAGGSLRLSCAASGRSFS

SEQ ID NO: 443 - protein, 5 aa, synthetic construct
TYAFG

SEQ ID NO: 444 - protein, 5 aa, synthetic construct
ATNMG

SEQ ID NO: 445 - protein, 5 aa, synthetic construct
IDIMG

SEQ ID NO: 446 - protein, 5 aa, synthetic construct
SYYMG

SEQ ID NO: 447 - protein, 14 aa, synthetic construct
WFRQAPGKEREFVA

SEQ ID NO: 448 - protein, 14 aa, synthetic construct
WFRQAPGKEREFVG

SEQ ID NO: 449 - protein, 14 aa, synthetic construct
WYRQTPGKEREWVA

SEQ ID NO: 450 - protein, 14 aa, synthetic construct
WFRQAPGKEREFVA

SEQ ID NO: 451 - protein, 17 aa, synthetic construct
TISQRGSTSYYSDSVEG

SEQ ID NO: 452 - protein, 17 aa, synthetic construct
IISESGRNTDYADSVKG

SEQ ID NO: 453 - protein, 16 aa, synthetic construct
TITGDDTTHYENSVKG

SEQ ID NO: 454 - protein, 17 aa, synthetic construct
GINWSGRSTHYADSVKG

SEQ ID NO: 455 - protein, 32 aa, synthetic construct
RFTISKDNAKSTVYLQMNSLQPEDTAVYYCAA

SEQ ID NO: 456 - protein, 32 aa, synthetic construct
RFTITRDNAKSTVYLQMNNLKPEDTAVYYCAL

SEQ ID NO: 457 - protein, 32 aa, synthetic construct
RFTISRDTAKNMVYLQMNSLEPEDTAVYYCNA

SEQ ID NO: 458 - protein, 32 aa, synthetic construct
RFTISRDNAKNTVYLQMNSLKPEDRAAYYCAA

SEQ ID NO: 459 - protein, 14 aa, synthetic construct
RYYGVDYRSTSYDF

SEQ ID NO: 460 - protein, 14 aa, synthetic construct
GRDYFATDTRQYNY

SEQ ID NO: 461 - protein, 8 aa, synthetic construct
GILPREEY

SEQ ID NO: 462 - protein, 19 aa, synthetic construct
DSAPSTRIGVRSLWDDYDY

SEQ ID NO: 463 - protein, 11 aa, synthetic construct
WGQGTLVTVSS

SEQ ID NO: 464 - protein, 11 aa, synthetic construct
WGLGTLVTVSS

SEQ ID NO: 465 - protein, 11 aa, synthetic construct
WGQGTLVTVSS

SEQ ID NO: 466 - protein, 11 aa, synthetic construct
WGQGTLVTVSS

SEQ ID NO: 467 - protein, 10 aa, synthetic construct
GFTFRSFGMS

SEQ ID NO: 468 - protein, 10 aa, synthetic construct
SISGSGSDTL

SEQ ID NO: 469 - protein, 7 aa, synthetic construct
GGSLSRS

SEQ ID NO: 470 - protein, 6 aa, synthetic construct
HHHHHH

(19) United States
(12) Patent Application Publication
(10) Pub. No.: US 2026/0274971 A1
(43) Pub. Date: Sep. 17, 2026

(54) IL-2RBETA GAMMA BASED LYSOSOMAL DEGRADER AND USES THEREOF

(71) Applicant: SANOFI, Paris (FR)

(72) Inventors: Bing LIU, Cambridge, MA (US); Xiangming LI, Winchester, MA (US); Qun ZHOU, Ashland, MA (US)

(21) Appl. No.: 19/426,841

(22) Filed: Dec. 19, 2025

(30) Foreign Application Priority Data
Dec. 20, 2024 (EP) 24307245.1

(51) Int. Cl. C07K 16/28; A61P 37/02; C07K 16/18

(52) U.S. Cl. CPC C07K 16/2866; A61P 37/02; C07K 16/18; C07K 16/2818; C07K 2317/31; C07K 2317/33; C07K 2317/52; C07K 2317/565; C07K 2317/567; C07K 2317/569; C07K 2317/622; C07K 2317/76; C07K 2317/77; C07K 2317/92; C07K 2317/94

(57) ABSTRACT

The present disclosure provides a method for degrading a target protein, comprising:
contacting an IL-2Rβγ positive cell with a multispecific binding protein, wherein the multispecific binding protein comprises: a) a first cell surface binding moiety that specifically binds to a CD122 (IL-2Rβ) subunit and/or a CD132 (IL-2Rγ) subunit, on the surface of the IL-2Rβγ positive cell, wherein the first cell surface binding moiety comprises at least one immunoglobulin domain or a fragment thereof; and b) a second binding moiety that is operatively linked to the first cell surface binding moiety and that specifically binds to the target protein, wherein specific binding of the multispecific binding protein to the IL-2Rβ subunit and/or the IL-2Rγ subunit facilitates the internalization of the target protein into the IL-2Rβγ positive cell. The present disclosure also provides multispecific binding proteins and methods of using the multispecific binding proteins to treat disease.

### RELATED APPLICATIONS

This application claims priority to European Patent Application Serial No. 24307245.1, filed on Dec. 20, 2024, the disclosure of which is hereby incorporated by reference.

### SEQUENCE LISTING

The instant application contains a Sequence Listing which has been submitted electronically in XML format and is hereby incorporated by reference in its entirety. Said XML file, created on May 18, 2026, is named 769749_SA9-390_ST26.xml and is 251,863 bytes in size.

### BACKGROUND

Over the past two decades, target protein degradation technologies have elicited great interest in expanding the landscape of druggable targets. It has also provided a unique mechanism of action for therapeutics as “event-driven” pharmacology as opposed to “occupancy-driven” associated with conventional inhibitors. For example, PROteolysis TArgeting Chimeras or PROTACs, small heterobifunctional molecules that form a ternary complex with an E3 ubiquitin ligase and a target of interest, resulting in target ubiquitination and degradation, have advanced through clinical trials. However, the therapeutic potential of PROTACs has been hampered by the poor permeability, pharmacokinetics and pharmacodynamic properties commonly seen with high molecular mass small molecules (over 1,000 Da). More recently, large molecule-based degrader technologies, such as lysosome targeting chimeras (LYTACs), have highlighted the potential of leveraging large molecules for targeted degradation of extracellular soluble and membrane-associated proteins. There is a need in the art for targeted degradation strategies, particularly those that are able to degrade targets in specific cell types.

### SUMMARY

The present disclosure provides a method for degrading a target protein, comprising contacting an IL-2Rβγ positive cell with a multispecific binding protein, wherein the multispecific binding protein comprises: a) a first cell surface binding moiety that specifically binds to a CD122 (IL-2Rβ) subunit and/or a CD132 (IL-2Rγ) subunit, on a surface of the IL-2Rβγ positive cell, wherein the first cell surface binding moiety comprises at least one immunoglobulin domain or a fragment thereof; and b) a second binding moiety that is operatively linked to the first cell surface binding moiety and that specifically binds to the target protein, wherein specific binding of the multispecific binding protein to the IL-2Rβ subunit and/or the IL-2Rγ subunit facilitates the internalization of the target protein into the IL-2Rβγ positive cell.

In one aspect the method of the disclosure comprises the IL-2Rβγ positive cell comprising either of a trimer consisting of the IL-2Rβ subunit, the IL-2Rγ subunit, and a CD25 (IL-2Rα) subunit (IL-2Rαβγ) or a dimer consisting of the IL-2Rβ subunit and the IL-2Rγ subunit (IL-2Rβγ). In one aspect, the target protein traffics to lysosomes for degradation of the target protein.

In one aspect, in the method of the disclosure the IL-2Rβγ positive cell is an immune cell, a white blood cell, or a hematopoietic cell. In one aspect, the IL-2Rβγ positive cell is a neoplastic cell. In one aspect, the IL-2Rβγ positive cell is a T-cell or a NK cell.

In one aspect, the method of the disclosure provides the first and second binding moiety form a heterodimer. In one aspect, the heterodimer comprises a fragment crystallizable (Fc) domain or variant thereof.

In one aspect, the method of the disclosure provides the first cell surface binding moiety binds to an IL-2Rβ subunit epitope. In one aspect, the first cell surface binding moiety binds to an IL-2Rγ subunit epitope. In one aspect, the first cell surface binding moiety binds to both an IL-2Rβ subunit epitope and an IL-2Rγ subunit epitope.

In one aspect, the IL-2Rβ subunit epitope and an IL-2Rγ subunit epitope are different epitopes. In one aspect, the IL-2Rβ subunit epitope and an IL-2Rγ subunit epitope are the same epitopes.

In one aspect, the method of the disclosure provides the multispecific binding protein exhibiting increased degradation of the target protein compared to a reference binding protein. In one aspect, the reference binding protein does not comprise the first cell surface binding moiety that specifically binds to the IL-2Rβ subunit, the IL-2Rγ subunit, or both IL-2Rβγ subunits but is otherwise identical to the multispecific binding protein. In one aspect, the multispecific binding protein exhibits increased degradation of the target protein by at least 2, 3, 4, 5, 10, 20, 30, 40, 50, 60, 70, 80, 90, or 100 percent compared to the reference binding protein. In one aspect, the multispecific binding protein degrades the target protein at least 2, 3, 4, 5, 10, 24, 48, or 72 hours faster compared to the reference binding protein. In one aspect, the multispecific binding protein's maximal amount of degradation (DMax) of the target protein is 55, 60, 65, 75, 80, 85, 90, 95, or 100 percent.

In one aspect, the method of the disclosure provides the first cell surface binding moiety binds an extracellular domain of the IL-2Rβ subunit, the IL-2Rγ subunit, or both IL-2Rβγ subunits. In one aspect, the first cell surface binding moiety comprises an IL-2Rβ subunit, an IL-2Rγ subunit, or both IL-2Rβγ subunits specific variable domain(s). In one aspect, the IL-2Rβ subunit, the IL-2Rγ subunit, or both IL-2Rβγ subunits specific variable domain(s) is operatively linked to a first Fc domain polypeptide. In one aspect, the first Fc domain polypeptide is an immunoglobulin G (IgG) Fc domain polypeptide.

In one aspect, the method of the disclosure provides the second binding moiety that specifically binds to the target protein comprises a target protein specific variable domain. In one aspect, the target protein specific variable domain is operatively linked to a second Fc domain polypeptide. In one aspect, the second Fc domain polypeptide is an immunoglobulin G (IgG) Fc domain polypeptide. In one aspect, the second binding moiety that specifically binds to the target protein comprises an antibody or an antigen binding fragment thereof. In one aspect, the antibody or an antigen binding fragment thereof comprises an Fc domain or a variant thereof.

In one aspect, the method of the disclosure provides the first and second IgG Fc domain polypeptides dimerize to form the multispecific binding protein. In one aspect, the first and second IgG Fc domain polypeptides dimerize by knobs-into-holes interactions, Fab arm exchange (FAE), electrostatic steering interactions, hydrophobic interactions, or any combination thereof. In one aspect, the first IgG Fc domain polypeptide comprises a knob substitution, and the second IgG Fc domain polypeptide comprises a hole substitution or wherein the first IgG Fc domain polypeptide comprises a hole substitution, and the second IgG Fc domain polypeptide comprises a knob substitution. In one aspect, the knob substitution is selected from the group consisting of arginine (R), phenylalanine (F), tyrosine (Y), and tryptophan (W). In one aspect, the hole substitution is selected from the group consisting of alanine (A), asparagine (N), aspartic acid (D), glycine (G), serine (S), threonine (T), and valine (V).

In one aspect, the method of the disclosure provides the first cell surface binding moiety and the second binding moiety of the multispecific binding moiety each independently comprises a VH, a Fab, a Fab′, a F(ab′)2, a variable fragment (Fv), a disulfide-stabilized Fv (dsFv), a single-chain Fv (scFv), a diabody, a triabody, an immunoglobulin single variable domain (ISVD), or an AFFIBODY®. In one aspect, the scFV is a linear scFV or a tandem scFV. In one aspect, the multispecific binding protein further comprises a Fc domain or a variant thereof. In one aspect, the ISVD is a VHH, such as humanized VHH, a camelized VH, a domain antibody (dAb) or a VNAR.

In one aspect, the method of the disclosure provides a multispecific binding protein comprise at least three immunoglobulin single variable domains (ISVDs),
wherein the at least three ISVDs comprise: a first ISVD that specifically binds to IL-2Rβ, a second ISVD that specifically binds IL-2Rγ, and a third ISVD that specifically binds to the target protein.

In one aspect, the method of the disclosure provides the target protein is a membrane-associated target protein, a soluble target protein, or both. In one aspect, the target protein is expressed on the surface of the same or a different IL-2Rβγ positive cell. In one aspect, the target protein is expressed on the surface of a neoplastic cell and/or an immune cell. In one aspect, the target protein is expressed on a T-cell. In one aspect, the T-cell is an activated T cell or a regulatory T (Treg) cell. In one aspect, the target protein is an immune checkpoint protein, a cancer antigen, and/or an immunomodulatory protein.

In one aspect, the method of the disclosure provides the immune checkpoint protein is a programmed death 1 (PD1), a programmed death-ligand 1 (PD-L1), or a protein that blocks the interaction between PD1 and PD-L1. In another aspect, the method of the disclosure provides the target protein is selected from the group consisting of: an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein. In certain aspects, the target protein is associated with a disease selected from the group consisting of: a neoplastic disorder, an autoimmune disease, an inflammatory disorder, an infectious disease, and a neurodegenerative disorder.

In one aspect, the method of the disclosure provides a multispecific binding protein comprises at least three ISVDs, wherein the at least three ISVDs comprise: a first ISVD that specifically binds to IL-2Rβ, a second ISVD that specifically binds IL-2Rγ, and a third ISVD that blocks PD1/PD-L1 interaction. In one aspect, the multispecific binding protein exhibits pH-dependent binding to the IL-2Rβ subunit, the IL-2Rγ subunit, the IL-2Rβγ dimer, or the IL-2Rαβγ trimer on the IL-2Rβγ positive cell. In one aspect, the multispecific binding protein exhibits pH-dependent binding to the target protein. In one aspect, the multispecific binding protein exhibits reduced binding at acidic pH.

In one aspect, the method of the disclosure provides the first cell surface binding moiety of the multispecific binding protein binds to the IL-2Rβ subunit, the IL-2Rγ subunit, the IL-2Rβγ dimer, or the IL-2Rαβγ trimer on the IL-2Rβγ positive cell with an affinity from about 100 pM to about 1 μM. In certain aspects, the second binding moiety of the multispecific binding protein binds to the target protein with an affinity from about 100 pM to about 1 μM.

In one aspect, the method of the disclosure provides a first ISVD comprises four framework regions (FR1-FR4) and three complementarity determining regions (CDR1-CDR3), according to Abm numbering, wherein: (i) the amino acid sequence of CDR1 is at least about 90% identical to an amino acid sequence selected from the group consisting of SEQ ID NOs: 114 and 338; or (ii) the amino acid sequence of CDR2 is at least about 90% identical an amino acid sequence selected from the group consisting of SEQ ID NOs:148 and 340; and (iii) the amino acid sequence of CDR3 is at least about 90% identical to an amino acid sequence selected from the group consisting of SEQ ID NOs: 182 and 342.

In certain aspects, the method of the disclosure provides (i) an amino acid sequence of CDR1 comprising an amino acid sequence selected from the group consisting of SEQ ID NOs: 114 and 338; (ii) an amino acid sequence of CDR2 comprising an amino acid sequence selected from the group consisting of SEQ ID NOs: 148 and 340; and (iii) an amino acid sequence of CDR3 comprising an amino acid sequence selected from the group consisting of SEQ ID NOs: 182 and 342.

In certain aspects, the method of the disclosure provides the CDR1, CDR2, and CDR3 amino acid sequences comprising: (i) the CDR1 corresponding to the amino acid sequence of SEQ ID NO: 114, a CDR2 corresponding to the amino acid sequence of SEQ ID NO: 148, and a CDR3 corresponding to the amino acid sequence of SEQ ID NO: 182; or (ii) the CDR1 corresponding to the amino acid sequence of SEQ ID NO: 338, a CDR2 corresponding to the amino acid sequence of SEQ ID NO: 340, and a CDR3 corresponding to the amino acid sequence of SEQ ID NO: 342.

In certain aspects, the method of the disclosure provides the second ISVD comprising four framework regions (FR1-FR4) and three complementarity determining regions (CDR1-CDR3), according to Abm numbering, wherein: (i) the amino acid sequence of CDR1 is at least about 90% identical to an amino acid sequence selected from the group consisting of SEQ ID NOs: 121 and 331; (ii) the amino acid sequence of CDR2 is at least about 90% identical to an amino acid sequence selected from the group consisting of SEQ ID NOs: 155 and 334; and (iii) the amino acid sequence of CDR3 is at least about 90% identical to an amino acid sequence selected from the group consisting of SEQ ID NOs: 189 and 336.

In certain aspects, the method of the disclosure provides (i) an amino acid sequence of CDR1 comprises an amino acid sequence selected from the group consisting of SEQ ID NOs: 121 and 331; (ii) an amino acid sequence of CDR2 comprises an amino acid sequence selected from the group consisting of SEQ ID NOs: 155 and 334; and (iii) an amino acid sequence of CDR3 comprises an amino acid sequence selected from the group consisting of SEQ ID NOs: 189 and 336.

In certain aspects, the method of the disclosure provides the CDR1, CDR2, and CDR3 amino acid sequences comprising: (i) the CDR1 corresponding to the amino acid sequence of SEQ ID NO: 121, a CDR2 corresponding to the amino acid sequence of SEQ ID NO: 155, and a CDR3 corresponding to the amino acid sequence of SEQ ID NO: 189; or (ii) the CDR1 corresponding to the amino acid sequence of SEQ ID NO: 331, a CDR2 corresponding to the amino acid sequence of SEQ ID NO: 334, and a CDR3 corresponding to the amino acid sequence of SEQ ID NO: 336.

In certain aspects, the method of the disclosure provides the first ISVD that specifically binds to IL-2Rβ comprising a sequence that has at least about 85%, about 90%, about 95%, or 100% identity to a sequence selected from the group consisting of SEQ ID Nos. 347 or 360.

In certain aspects, the method of the disclosure provides the second ISVD that specifically binds to IL-2Rγ comprising a sequence that has at least about 85%, about 90%, about 95%, or 100% identity to a sequence selected from the group consisting of SEQ ID Nos. 354 or 359.

In certain aspects, the method of the disclosure provides the third ISVD that blocks PD1/PD-L1 interaction comprising a sequence that has at least about 85%, about 90%, about 95%, or 100% identity to SEQ ID NO: 361. In some aspects, the third ISVD that blocks PD1/PD-L1 interaction comprises a sequence that has at least about 85%, about 90%, about 95%, or 100% identity to SEQ ID NO: 362. In some aspects, the third ISVD that blocks PD1/PD-L1 interaction comprises a sequence that has at least about 85%, about 90%, about 95%, or 100% identity to SEQ ID NO: 363.

In certain aspects, the method of the disclosure provides a multivalent ISVD construct comprising a sequence that has at least about 85%, about 90%, about 95%, or 100% identity to anyone of SEQ ID NOs: 364-386.

In certain aspects, the method of the disclosure provides the multispecific binding protein comprises a human serum albumin binding domain. In certain aspects, the human serum albumin domain comprises a sequence selected from the group consisting of SEQ ID Nos. 77-91 and 424-433.

In certain aspects, the method of the disclosure provides the multispecific binding protein comprises an amino acid sequence set forth in SEQ ID NO: 60-63. The multispecific binding protein comprises an amino acid sequence that has at least about 85%, about 90%, about 95%, or 100% identity to SEQ ID NO: 422 or 423. In certain aspects, the multispecific binding protein comprises one or more mutations or glycan modifications to modulate Fc mediated effector function. In some aspects, the multispecific binding protein comprises one or more mutations to modulate serum half-life.

In one aspect, the disclosure provides a multispecific binding protein comprising: a) a first cell surface binding moiety that specifically binds to a CD122 (IL-2Rβ) subunit and/or a CD132 (IL-2Rγ) subunit, on the surface of an IL-2Rβγ positive cell, wherein the first cell surface binding moiety comprises an immunoglobulin domain; and b) a second binding moiety that is operatively linked to the first cell surface binding moiety and that specifically binds to a target protein, wherein specific binding of the multispecific binding protein to the IL-2Rβ subunit and/or the IL-2Rγ subunit facilitates internalization of the target protein bound to the multispecific binding protein into the IL-2Rβγ positive cell.

In one aspect, the disclosure provides the IL-2Rβγ positive cell comprises either of a trimer consisting of the IL-2Rβ subunit, the IL-2Rγ subunit, and an IL-2Rα subunit (IL-2Rαβγ) or a dimer consisting of the IL-2Rβ subunit and the IL-2Rγ subunit (IL-2Rβγ).

In one aspect, the target protein traffics to lysosomes for degradation of the target protein.

In one aspect, the disclosure provides the IL-2Rβγ positive cell is an immune cell, a white blood cell, or a hematopoietic cell. In another aspect, the IL-2Rβγ positive cell is a neoplastic cell. In certain aspects, the IL-2Rβγ positive cell is a T-cell or a NK cell.

In one aspect, the disclosure provides, the first and second binding moiety form a heterodimer. In another aspect, the heterodimer comprises a fragment crystallizable (Fc) domain or variant thereof.

In another aspect, the disclosure provides a multispecific binding protein comprising a first cell surface binding moiety binds to an IL-2Rβ subunit epitope. In one aspect, the first cell surface binding moiety binds to an IL-2Rγ subunit epitope. In one aspect, the first cell surface binding moiety binds to both an IL-2Rβ epitope and an IL-2Rγ subunit epitope. In one aspect, the IL-2Rβ epitope and the IL-2Rγ subunit epitope are different epitopes. In one aspect, the IL-2Rβ epitope and the IL-2Rγ subunit epitope are the same epitopes.

In another aspect, the disclosure provides a multispecific binding protein wherein the first cell surface binding moiety binds an extracellular domain of the IL-2Rβ subunit, the IL-2Rγ subunit, or both the IL-2Rβγ subunits. In one aspect, the first cell surface binding moiety comprises an IL-2Rβ subunit, an IL-2Rγ subunit, or both IL-2Rβγ subunits specific variable domain(s). In another aspect, the IL-2Rβ subunit, the IL-2Rγ subunit, or both IL-2Rβγ subunits specific variable domain is operatively linked to a first Fc domain polypeptide. In one aspect, the first Fc domain polypeptide is an immunoglobulin G (IgG) Fc domain polypeptide.

In one aspect, the disclosure provides a multispecific binding protein comprising the second binding moiety that specifically binds to the target protein comprises a target protein specific variable domain. In one aspect, the target protein specific variable domain is operatively linked to a second Fc domain polypeptide. In one aspect, the second Fc domain polypeptide is an immunoglobulin G (IgG) Fc domain polypeptide. In one aspect, the second binding moiety that specifically binds to the target protein comprises an antibody or an antigen binding fragment thereof.

In one aspect, the disclosure provides a multispecific binding protein comprising a first and second IgG Fc domain polypeptides dimerize to form the multispecific binding protein. In one aspect, the first and second IgG Fc domain polypeptides dimerize by knobs-into-holes interactions, Fab arm exchange (FAE), electrostatic steering interactions, hydrophobic interactions, or any combination thereof. In one aspect, the first IgG Fc domain polypeptide comprises a knob substitution, and the second IgG Fc domain polypeptide comprises a hole substitution or wherein the first IgG Fc domain polypeptide comprises a hole substitution, and the second IgG Fc domain polypeptide comprises a knob substitution. In one aspect, the knob substitution is selected from the group consisting of arginine (R), phenylalanine (F), tyrosine (Y), and tryptophan (W). In one aspect, the hole substitution is selected from the group consisting of alanine (A), asparagine (N), aspartic acid (D), glycine (G), serine (S), threonine (T), and valine (V).

In one aspect, the disclosure provides a multispecific binding protein comprising the first cell surface binding moiety and the second binding moieties of the multispecific binding moiety each independently comprises a VH, a Fab, a Fab′, a F(ab′)2, a variable fragment (Fv), a disulfide-stabilized Fv (dsFv), a single-chain Fv (scFv), a diabody, a triabody, an immunoglobulin single variable domain (ISVD), or an AFFIBODY®. In one aspect, the scFV is a linear scFV or a tandem scFV. In one aspect, the ISVD is a VHH, humanized VHH, a camelized VH, a domain antibody (dAb), or a VNAR.

In one aspect, a multispecific binding protein comprises at least three ISVDs, wherein the at least three ISVDs comprise: a first ISVD that specifically binds to IL-2Rβ, a second ISVD that specifically binds IL-2Rγ, and a third ISVD that specifically binds to the target protein. In one aspect, the multispecific binding protein further comprises a Fc domain or a variant thereof.

In one aspect, the target protein is a membrane-associated target protein, a soluble target protein, or both. In one aspect, the target protein is expressed on the surface of the same or a different IL-2Rβγ positive cell. In one aspect, the target protein is expressed on the surface of a neoplastic cell and/or an immune cell. In one aspect, the target protein is expressed on a T-cell. In one aspect, the T-cell is an activated T cell or a regulatory T (Treg) cell. In one aspect, the target protein is an immune checkpoint protein, a cancer antigen, and/or an immunomodulatory protein. In one aspect, the immune checkpoint protein is PD1, PD-L1, or a protein that blocks the interaction between PD1 and PD-L1. In one aspect, the target protein is selected from the group consisting of: an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein. In one aspect, the target protein is associated with a disease selected from the group consisting of: a neoplastic disorder, an autoimmune disease, an inflammatory disorder, an infectious disease, and a neurodegenerative disorder.

In one aspect, a multispecific binding protein of the disclosure comprises at least three ISVDs, wherein the at least three ISVDs comprise: a first ISVD that specifically binds to IL-2Rβ, a second ISVD that specifically binds IL-2Rγ, and a third ISVD that blocks PD1/PD-L1 interaction.

In one aspect, a multispecific binding protein of the disclosure comprises a combination of the first and the second ISVD has an agonistic effect on IL-2Rβγ. In one aspect, the two subunits of the IL-2Rβ and IL-2Rγ comprise amino acid sequences corresponding to amino acid sequences of SEQ ID NO: 390 and SEQ ID NO: 391, respectively.

In one aspect, a multispecific binding protein of the disclosure comprises a third ISVD that specifically binds to PD-1.

In one aspect, a multispecific binding protein of the disclosure comprises a first ISVD comprising four framework regions (FR1-FR4) and three complementarity determining regions (CDR1-CDR3), according to Abm numbering, wherein: (i) the amino acid sequence of CDR1 is at least about 90% identical to an amino acid sequence selected from the group consisting of SEQ ID NOs: 114 and 338; or (ii) the amino acid sequence of CDR2 is at least about 90% identical to an amino acid sequence selected from the group consisting of SEQ ID NOs:148 and 340; and (iii) the amino acid sequence of CDR3 is at least about 90% identical to an amino acid sequence selected from the group consisting of SEQ ID NOs: 182 and 342.

In one aspect, a multispecific binding protein of the disclosure comprises: (i) an amino acid sequence of CDR1 comprising an amino acid sequence selected from the group consisting of SEQ ID NOs: 114 and 338; (ii) an amino acid sequence of CDR2 comprising an amino acid sequence selected from the group consisting of SEQ ID NOs: 148 and 340; and (iii) an amino acid sequence of CDR3 comprising an amino acid sequence selected from the group consisting of SEQ ID NOs: 182 and 342.

In one aspect, a multispecific binding protein of the disclosure comprises the CDR1, CDR2, and CDR3 amino acid sequences comprising: (i) the CDR1 corresponding to the amino acid sequence of SEQ ID NO: 114, a CDR2 corresponding to the amino acid sequence of SEQ ID NO: 148, and a CDR3 corresponding to the amino acid sequence of SEQ ID NO: 182; or (ii) the CDR1 corresponding to the amino acid sequence of SEQ ID NO: 338, a CDR2 corresponding to the amino acid sequence of SEQ ID NO: 340, and a CDR3 corresponding to the amino acid sequence of SEQ ID NO: 342.

In one aspect, a multispecific binding protein of the disclosure comprises a second ISVD comprising four framework regions (FR1-FR4) and three complementarity determining regions (CDR1-CDR3), according to Abm numbering, wherein: (i) the amino acid sequence of CDR1 is at least about 90% identical to an amino acid sequence selected from the group consisting of SEQ ID NOs: 121 and 331; (ii) the amino acid sequence of CDR2 is at least about 90% identical to an amino acid sequence selected from the group consisting of SEQ ID NOs: 155 and 334; and (iii) the amino acid sequence of CDR3 is at least about 90% identical to an amino acid sequence selected from the group consisting of SEQ ID NOs: 189 and 336.

In one aspect, a multispecific binding protein of the disclosure comprises (i) an amino acid sequence of CDR1 comprising an amino acid sequence selected from the group consisting of SEQ ID NOs: 121 and 331; (ii) an amino acid sequence of CDR2 comprising an amino acid sequence selected from the group consisting of SEQ ID NOs: 155 and 334; and (iii) an amino acid sequence of CDR3 comprising an amino acid sequence selected from the group consisting of SEQ ID NOs: 189 and 336.

In one aspect, a multispecific binding protein of the disclosure comprises a CDR1, CDR2, and CDR3 amino acid sequence comprising: (i) the CDR1 corresponding to the amino acid sequence of SEQ ID NO: 121, a CDR2 corresponding to the amino acid sequence of SEQ ID NO: 155, and a CDR3 corresponding to the amino acid sequence of SEQ ID NO: 189; or (ii) the CDR1 corresponding to the amino acid sequence of SEQ ID NO: 331, a CDR2 corresponding to the amino acid sequence of SEQ ID NO: 334, and a CDR3 corresponding to the amino acid sequence of SEQ ID NO: 336.

In one aspect, a multispecific binding protein of the disclosure comprises the first ISVD that specifically binds to IL-2Rβ and comprises a sequence that has at least about 85%, about 90%, about 95%, or 100% identity to a sequence selected from the group consisting of SEQ ID Nos. 347 or 360.

In one aspect, a multispecific binding protein of the disclosure comprises the second ISVD that specifically binds to IL-2Rγ and comprises a sequence that has at least about 85%, about 90%, about 95%, or 100% identity to a sequence selected from the group consisting of SEQ ID Nos. 354 or 359.

In one aspect, a multispecific binding protein of the disclosure comprises the third ISVD that blocks PD1/PD-L1 interaction and comprises a sequence that has at least about 85%, about 90%, about 95%, or 100% identity to SEQ ID NO: 361. In some aspects, the third ISVD that blocks PD1/PD-L1 interaction comprises a sequence that has at least about 85%, about 90%, about 95%, or 100% identity to SEQ ID NO: 362.

In some aspects, the third ISVD that blocks PD1/PD-L1 interaction comprises a sequence that has at least about 85%, about 90%, about 95%, or 100% identity to SEQ ID NO: 363.

In certain aspects, a multispecific binding protein of the disclosure comprises a sequence that has at least about 85%, about 90%, about 95%, or 100% identity to any one of SEQ ID NOs: 364-386.

In one aspect, a multispecific binding protein of the disclosure comprises a human serum albumin binding domain. In one aspect, the human serum albumin domain comprises a sequence selected from the group consisting of SEQ ID Nos. 77-91 and 424-434.

In one aspect, a multispecific binding protein of the disclosure comprises an amino acid sequence set forth in SEQ ID NO: 60-63. In one aspect, a multispecific binding protein of the disclosure comprises an amino acid sequence that has at least about 85%, about 90%, about 95%, or 100% identity to SEQ ID NO: 422 or 423.

In one aspect, a multispecific binding protein of the disclosure comprises the relative order of the first, the second, and the third ISVD within the multispecific binding protein from the N-(N) to C-(C) terminal domain are selected from the following orders:
(i): (N)-first ISVD-second ISVD-third ISVD-(C);
(ii): (N)-second ISVD-first ISVD-third ISVD-(C);
(iii): (N)-third ISVD-first ISVD-second ISVD-(C);
(iv): (N)-third ISVD-second ISVD-first ISVD-(C);
(v): (N)-first ISVD-third ISVD-second ISVD-(C); or
(vi): (N)-second ISVD-third ISVD-first ISVD-(C).

In one aspect, said multispecific binding protein further comprises one or more binding moieties operatively linked to the first, second, or third ISVD. In one aspect, the operative linker is a peptide linker. In one aspect, the peptide linker is at 90% identical to a peptide linker encoded by an amino acid sequence set forth in SEQ ID NOs: 1-8, 56-58 or 64-76.

In one aspect, a multispecific binding protein of the disclosure comprises one or more binding moieties (or units) that increases half-life compared to a reference multispecific binding protein without said one or more binding moieties. In one aspect, the one or more binding moieties facilitates binding to a FcRn. In one aspect, the binding unit that facilitates binding to a FcRn is selected from a group consisting of: a Fc constant domain, a modified Fc domain, a serum albumin, a serum albumin binding moiety/unit, a serum immunoglobulin, or a variant thereof. In one aspect, the serum albumin is a human serum albumin or the serum immunoglobulin is an IgG. In one aspect, the binding unit is an ISVD that binds to the human serum albumin. In one aspect, the ISVD that binds to human serum albumin is located proximal to the N- or C-terminal end of the multispecific binding protein. In one aspect, an amino acid sequence encoding the ISVD that binds to human serum albumin exhibits a sequence identity of more than 90% with an amino acid sequence selected from the group consisting of SEQ ID NOs: 77-91 or 424-433. In one aspect, the amino acid sequence is selected from the group consisting of SEQ ID NOs: 77, 78, 81, 83, and 90.

In one aspect, a multispecific binding protein of the disclosure exhibits pH-dependent binding to IL-2Rβγ on the IL-2Rβγ positive cell. In one aspect, the multispecific binding protein exhibits pH-dependent binding to the target protein. In one aspect, the multispecific binding protein exhibits reduced binding at acidic pH. In one aspect, the first cell surface binding moiety of the multispecific binding protein binds to IL-2Rβγ on the IL-2Rβγ positive cell with an affinity from about 100 pM to about 1 μM. In one aspect, the second binding moiety of the multispecific binding protein binds to the target protein with an affinity from about 100 pM to about 1 μM. In one aspect, the multispecific binding protein comprises one or more mutations or glycan modifications to modulate Fc mediated effector function. In one aspect, the multispecific binding protein comprises one or more mutations to modulate serum half-life.

In one aspect, a multispecific binding protein of the disclosure comprises an amino acid sequence that has at least about 85%, about 90%, about 95%, or 100% identity SEQ ID NO: 60-63. In one aspect, a multispecific binding protein of the disclosure comprises an amino acid sequence that has at least about 85%, about 90%, about 95%, or 100% identity to SEQ ID NO: 422 or 423.

In one aspect, a pharmaceutical composition comprises a multispecific binding protein of the disclosure and a pharmaceutically acceptable carrier or diluent. In one aspect, a nucleic acid molecule encodes a multispecific binding protein of the disclosure. In one aspect, a vector comprises said nucleic acid molecule. In one aspect, a cell comprises said nucleic acid molecule or said vector.

In one aspect, a method of depleting a target protein comprises administering to a subject an effective amount of a multispecific binding protein of the disclosure or the pharmaceutical composition comprising a multispecific binding protein of the disclosure. In one aspect, the multispecific binding protein is internalized by the IL-2Rβγ positive cell. In one aspect, an amount of multispecific binding protein internalized by the IL-2Rβγ positive cell is greater than an amount of a reference binding protein that does not comprise the first cell surface binding moiety. In one aspect, the target protein is selectively depleted from a target tissue or circulation of the subject. In one aspect, administering the multispecific binding protein results in at least about 10%, 20, 30%, 40%, 50%, 75%, or 90% depletion of the target protein from the target tissue or circulation of the subject.

In one aspect, the disclosure provides a method of treating a disease comprising administering an effective amount of a multispecific binding protein of the disclosure or a pharmaceutical composition comprising a multispecific binding protein of the disclosure. In some aspects, the disease is selected from a group consisting of: a neoplastic disorder, an autoimmune disease, an inflammatory disorder, an infectious disease, or a neurodegenerative disorder. In some aspects, said composition is used in a treatment of a checkpoint refractory solid tumor.

In one aspect, the disclosure provides a method for producing a multispecific binding protein of the disclosure, said method at least comprising the steps of: a) expressing, in a suitable host cell or host organism or in another suitable expression system, a nucleic acid expressing the multispecific binding protein; optionally followed by: b) isolating and/or purifying the multispecific binding protein.

In one aspect, the disclosure provides an immunoglobulin single variable domain (ISVD) specifically binding human IL2Rgamma, that essentially consists of 4 framework regions (FR1 to FR4, respectively) and 3 complementarity determining regions (CDR1 to CDR3, respectively), in which: CDR1 (AbM numbering) has an amino acid sequence selected from: a) the amino acid sequence of SEQ ID NO: 121; b) amino acid sequences that have at least 80% amino acid identity with the amino acid sequence of SEQ ID NO: 121; and c) amino acid sequences that have 3, 2, or 1 amino acid difference with the amino acid sequences of SEQ ID NO: 121; and CDR2 (AbM numbering) has an amino acid sequence selected from: d) the amino acid sequence of SEQ ID NO: 155; e) amino acid sequences that have at least 80% amino acid identity with the amino acid sequence of SEQ ID NO: 155; f) amino acid sequences that have 3, 2, or 1 amino acid difference with the amino acid sequence of SEQ ID NO: 155; and CDR3 (AbM numbering) has an amino acid sequence selected from: g) the amino acid sequence of SEQ ID NO: 189; h) amino acid sequences that have at least 80% amino acid identity with the amino acid sequence of SEQ ID NO: 189; i) amino acid sequences that have 3, 2, or 1 amino acid difference with the amino acid sequence of SEQ ID NO: 189.

In certain embodiments, the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 354.

In certain embodiments, CDR1 consists of the amino acid sequence of SEQ ID NO: 121; CDR2 consists of the amino acid sequences of SEQ ID NO: 155; and CDR3 consists of the amino acid sequence of SEQ ID NO: 189.

In one aspect, the disclosure provides an immunoglobulin single variable domain (ISVD) specifically binding human IL2Rbeta, that essentially consists of 4 framework regions (FR1 to FR4, respectively) and 3 complementarity determining regions (CDR1 to CDR3, respectively), in which: CDR1 (AbM numbering) has an amino acid sequence selected from: a) the amino acid sequence of SEQ ID NO: 114; b) amino acid sequences that have at least 80% amino acid identity with the amino acid sequence of SEQ ID NO: 114; and c) amino acid sequences that have 3, 2, or 1 amino acid difference with the amino acid sequences of SEQ ID NO: 114; and CDR2 (AbM numbering) has an amino acid sequence selected from: d) the amino acid sequence of SEQ ID NO: 148; e) amino acid sequences that have at least 80% amino acid identity with the amino acid sequence of SEQ ID NO: 148; f) amino acid sequences that have 3, 2, or 1 amino acid difference with the amino acid sequence of SEQ ID NO: 148; and CDR3 (AbM numbering) has an amino acid sequence selected from: g) the amino acid sequence of SEQ ID NO: 182; h) amino acid sequences that have at least 80% amino acid identity with the amino acid sequence of SEQ ID NO: 182; g) amino acid sequences that have 3, 2, or 1 amino acid difference with the amino acid sequence of SEQ ID NO: 182.

In certain embodiments, the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NOs: 347.

In certain embodiments, CDR1 consists of the amino acid sequence of SEQ ID NO: 114; CDR2 consists of the amino acid sequences of SEQ ID NO: 148; and CDR3 consists of the amino acid sequence of SEQ ID NO: 182.

In one aspect, the disclosure provides an immunoglobulin single variable domain (ISVD) specifically binding human IL2Rgamma, that essentially consists of 4 framework regions (FR1 to FR4, respectively) and 3 complementarity determining regions (CDR1 to CDR3, respectively), in which: CDR1 (AbM numbering) has an amino acid sequence selected from: a) the amino acid sequence of SEQ ID NO: 331; b) amino acid sequences that have at least 80% amino acid identity with the amino acid sequence of SEQ ID NO: 331; and c) amino acid sequences that have 3, 2, or 1 amino acid difference with the amino acid sequences of SEQ ID NO: 331; and CDR2 (AbM numbering) has an amino acid sequence selected from: d) the amino acid sequence of SEQ ID NO: 334; e) amino acid sequences that have at least 80% amino acid identity with the amino acid sequence of SEQ ID NO: 334; f) amino acid sequences that have 3, 2, or 1 amino acid difference with the amino acid sequence of SEQ ID NO: 334; and CDR3 (AbM numbering) has an amino acid sequence selected from: g) the amino acid sequence of SEQ ID NO: 336; h) amino acid sequences that have at least 80% amino acid identity with the amino acid sequence of SEQ ID NO: 336; i) amino acid sequences that have 3, 2, or 1 amino acid difference with the amino acid sequence of SEQ ID NO: 336.

In certain embodiments, the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 359.

In certain embodiments, CDR1 consists of the amino acid sequence of SEQ ID NO: 331; CDR2 consists of the amino acid sequences of SEQ ID NO: 334; and CDR3 consists of the amino acid sequence of SEQ ID NO: 336.

In one aspect, the disclosure provides an immunoglobulin single variable domain (ISVD) specifically binding human IL2Rbeta, that essentially consists of 4 framework regions (FR1 to FR4, respectively) and 3 complementarity determining regions (CDR1 to CDR3, respectively), in which: CDR1 (AbM numbering) has an amino acid sequence selected from: a) the amino acid sequence of SEQ ID NO: 338; b) amino acid sequences that have at least 80% amino acid identity with the amino acid sequence of SEQ ID NO: 338; and c) amino acid sequences that have 3, 2, or 1 amino acid difference with the amino acid sequences of SEQ ID NO: 338; and CDR2 (AbM numbering) has an amino acid sequence selected from: d) the amino acid sequence of SEQ ID NO: 340; e) amino acid sequences that have at least 80% amino acid identity with the amino acid sequence of SEQ ID NO: 340; f) amino acid sequences that have 3, 2, or 1 amino acid difference with the amino acid sequence of SEQ ID NO: 340; and CDR3 (AbM numbering) has an amino acid sequence selected from: g) the amino acid sequence of SEQ ID NO: 342; h) amino acid sequences that have at least 80% amino acid identity with the amino acid sequence of SEQ ID NO: 342; i) amino acid sequences that have 3, 2, or 1 amino acid difference with the amino acid sequence of SEQ ID NO: 342.

In certain embodiments, the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 360.

In certain embodiments, CDR1 consists of the amino acid sequence of SEQ ID NO: 338; CDR2 consists of the amino acid sequences of SEQ ID NO: 340; and CDR3 consists of the amino acid sequence of SEQ ID NO: 342.

In one aspect, the disclosure provides an immunoglobulin single variable domain (ISVD) specifically binding human IL2Rgamma, that essentially consists of 4 framework regions (FR1 to FR4, respectively) and 3 complementarity determining regions (CDR1 to CDR3, respectively), in which: CDR1 (Kabat numbering) has an amino acid sequence selected from: a) the amino acid sequence of SEQ ID NO:443; b) amino acid sequences that have at least 80% amino acid identity with the amino acid sequence of SEQ ID NO: 443; c) amino acid sequences that have 3, 2, or 1 amino acid difference with the amino acid sequences of SEQ ID NO: 443; and CDR2 (Kabat numbering) has an amino acid sequence selected from: d) the amino acid sequence of SEQ ID NO: 451; e) amino acid sequences that have at least 80% amino acid identity with the amino acid sequence of SEQ ID NO: 451; f) amino acid sequences that have 3, 2, or 1 amino acid difference with the amino acid sequence of SEQ ID NO: 451; and CDR3 (Kabat numbering) has an amino acid sequence selected from: g) the amino acid sequence of SEQ ID NO: 459; h) amino acid sequences that have at least 80% amino acid identity with the amino acid sequence of SEQ ID NO: 459; i) amino acid sequences that have 3, 2, or 1 amino acid difference with the amino acid sequence of SEQ ID NO: 459.

In certain embodiments, the amino acid sequences of the CDRs (Kabat numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 354.

In certain embodiments, CDR1 consists of the amino acid sequence of SEQ ID NO: 443; CDR2 consists of the amino acid sequences of SEQ ID NO: 451; and CDR3 consists of the amino acid sequence of SEQ ID NO: 459.

In one aspect, the disclosure provides an immunoglobulin single variable domain (ISVD) specifically binding human IL2Rbeta, that essentially consists of 4 framework regions (FR1 to FR4, respectively) and 3 complementarity determining regions (CDR1 to CDR3, respectively), in which: CDR1 (Kabat numbering) has an amino acid sequence selected from: a) the amino acid sequence of SEQ ID NO: 444; b) amino acid sequences that have at least 80% amino acid identity with the amino acid sequence of SEQ ID NO: 444; c) amino acid sequences that have 3, 2, or 1 amino acid difference with the amino acid sequences of SEQ ID NO: 444; and CDR2 (Kabat numbering) has an amino acid sequence selected from: d) the amino acid sequence of SEQ ID NO: 452; e) amino acid sequences that have at least 80% amino acid identity with the amino acid sequence of SEQ ID NO: 452; f) amino acid sequences that have 3, 2, or 1 amino acid difference with the amino acid sequence of SEQ ID NO: 452; and CDR3 (Kabat numbering) has an amino acid sequence selected from: g) the amino acid sequence of SEQ ID NO: 460; h) amino acid sequences that have at least 80% amino acid identity with the amino acid sequence of SEQ ID NO: 460; i) amino acid sequences that have 3, 2, or 1 amino acid difference with the amino acid sequence of SEQ ID NO: 460.

In certain embodiments, the amino acid sequences of the CDRs (Kabat numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 347.

In certain embodiments, CDR1 consists of the amino acid sequence of SEQ ID NO: 444; CDR2 consists of the amino acid sequences of SEQ ID NO: 452; and CDR3 consists of the amino acid sequence of SEQ ID NO: 460.

In one aspect, the disclosure provides an immunoglobulin single variable domain (ISVD) specifically binding human IL2Rgamma, that essentially consists of 4 framework regions (FR1 to FR4, respectively) and 3 complementarity determining regions (CDR1 to CDR3, respectively), in which: CDR1 (Kabat numbering) has an amino acid sequence selected from: a) the amino acid sequence of SEQ ID NO: 445; b) amino acid sequences that have at least 80% amino acid identity with the amino acid sequence of SEQ ID NO: 445; c) amino acid sequences that have 3, 2, or 1 amino acid difference with the amino acid sequences of SEQ ID NO: 445; and CDR2 (Kabat numbering) has an amino acid sequence selected from: d) the amino acid sequence of SEQ ID NO: 453; e) amino acid sequences that have at least 80% amino acid identity with the amino acid sequence of SEQ ID NO: 453; f) amino acid sequences that have 3, 2, or 1 amino acid difference with the amino acid sequence of SEQ ID NO: 453; and CDR3 (Kabat numbering) has an amino acid sequence selected from: g) the amino acid sequence of SEQ ID NO: 461; h) amino acid sequences that have at least 80% amino acid identity with the amino acid sequence of SEQ ID NO: 461; i) amino acid sequences that have 3, 2, or 1 amino acid difference with the amino acid sequence of SEQ ID NO: 461.

In certain embodiments, the amino acid sequences of the CDRs (Kabat numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 359.

In certain embodiments, CDR1 consists of the amino acid sequence of SEQ ID NO: 445; CDR2 consists of the amino acid sequences of SEQ ID NO: 453; and CDR3 consists of the amino acid sequence of SEQ ID NO: 461.

In one aspect, the disclosure provides an immunoglobulin single variable domain (ISVD) specifically binding human IL2Rbeta, that essentially consists of 4 framework regions (FR1 to FR4, respectively) and 3 complementarity determining regions (CDR1 to CDR3, respectively), in which: CDR1 (Kabat numbering) has an amino acid sequence selected from: a) the amino acid sequence of SEQ ID NO: 446; b) amino acid sequences that have at least 80% amino acid identity with the amino acid sequence of SEQ ID NO: 446; c) amino acid sequences that have 3, 2, or 1 amino acid difference with the amino acid sequences of SEQ ID NO: 446; and CDR2 (Kabat numbering) has an amino acid sequence selected from: d) the amino acid sequence of SEQ ID NO: 454; e) amino acid sequences that have at least 80% amino acid identity with the amino acid sequence of SEQ ID NO: 454; f) amino acid sequences that have 3, 2, or 1 amino acid difference with the amino acid sequence of SEQ ID NO: 454; and CDR3 (Kabat numbering) has an amino acid sequence selected from: g) the amino acid sequence of SEQ ID NO: 462; h) amino acid sequences that have at least 80% amino acid identity with the amino acid sequence of SEQ ID NO: 462; i) amino acid sequences that have 3, 2, or 1 amino acid difference with the amino acid sequence of SEQ ID NO: 462.

In certain embodiments, the amino acid sequences of the CDRs (Kabat numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 360.

In certain embodiments, CDR1 consists of the amino acid sequence of SEQ ID NO: 446; CDR2 consists of the amino acid sequences of SEQ ID NO: 454; and CDR3 consists of the amino acid sequence of SEQ ID NO: 462.

In certain embodiments, the amino acid sequences of the CDRs (Chothia definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360.

In certain embodiments, the amino acid sequences of the CDRs (IMGT definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360.

In certain embodiments, the amino acid sequence i) has 80% amino acid sequence identity with one of the amino acid sequences of SEQ ID NOs: 354, 347, 359, and 360, in which for the purposes of determining the degree of amino acid identity, the amino acid residues that form the CDR sequences are disregarded; and in which: ii) preferably one or more of the amino acid residues at positions 11, 37, 44, 45, 47, 83, 84, 103, 104 and 108 according to the Kabat numbering are chosen from the Hallmark residues mentioned in Table 1.

In certain embodiments, the ISVD consists essentially of a heavy chain variable domain sequence that is derived from a conventional four-chain antibody or that consists essentially of a heavy chain variable domain sequence that is derived from heavy chain antibody.

In certain embodiments, the ISVD consists essentially of a VHH, a humanized VHH, a camelized VH, a domain antibody, a single domain antibody, or a dAb.

In certain embodiments, the amino acid sequence is chosen from the group consisting of SEQ ID NOs: 354, 347, 359, and 360 or from the group of amino acid sequences that have more than 80%, preferably more than 90%, more preferably more than 95%, such as 99% or more amino acid sequence identity with at least one amino acid sequence selected from SEQ ID NOs: 354, 347, 359, and 360.

In certain embodiments, the ISVD specifically binds to human and cyno IL2R-gamma and/or IL2R-beta and does not bind to IL21R or other members of the IL2R family.

In certain embodiments, the ISVD antagonizes an activity of IL2RBeta and/or IL2Rgamma.

In one aspect, the disclosure provides a polypeptide or construct that comprises or essentially consists of one or more ISVDs described herein, and optionally further comprises one or more other groups, residues, moieties or binding units, optionally linked via one or more linkers.

In certain embodiments, said one or more other groups, residues, moieties or binding units are amino acid sequences.

In certain embodiments, said one or more linkers are one or more amino acid sequences.

In certain embodiments, said one or more other groups, residues, moieties or binding units are immunoglobulin sequences.

In certain embodiments, said one or more other groups, residues, moieties or binding units are ISVDs.

In certain embodiments, said one or more other groups, residues, moieties or binding units are chosen from the group consisting of VHHs, humanized VHHs, camelized VHs, domain antibodies, single domain antibodies and dAbs.

In certain embodiments, the polypeptide or construct is a multivalent construct.

In certain embodiments, the polypeptide or construct is a multispecific construct.

In certain embodiments, said one or more other groups, residues, moieties or binding units provide the polypeptide or construct with increased half-life, compared to the ISVD without the one or more other groups, residues, moieties or binding units.

In certain embodiments, said one or more other groups, residues, moieties or binding units that provide the polypeptide or construct with increased half-life is chosen from the group consisting of a polyethylene glycol molecule (PEG), serum proteins or fragments thereof, binding units that specifically bind to serum proteins, an Fc portion, and small proteins or peptides that specifically bind to serum proteins.

In certain embodiments, said one or more other groups, residues, moieties or binding units that provide the polypeptide or construct with increased half-life is chosen from the group consisting of human serum albumin or fragments thereof.

In certain embodiments, said one or more other groups, residues, moieties or binding units that provides the polypeptide or construct with increased half-life are chosen from the group consisting of binding units that specifically bind to serum albumin (such as human serum albumin) or a serum immunoglobulin (such as IgG).

In certain embodiments, said one or more other groups, residues, moieties or binding units that provides the polypeptide or construct with increased half-life are chosen from the group consisting of VHHs, humanized VHHs, camelized VHs, domain antibodies, single domain antibodies, or dAbs that specifically bind to serum albumin (such as human serum albumin) or a serum immunoglobulin (such as IgG).

In certain embodiments, said one or more other groups, residues, moieties or binding units that provides the polypeptide or construct with increased half-life is an ISVD that specifically binds human serum albumin.

In certain embodiments, said ISVD that specifically binds human serum albumin essentially consists of 4 framework regions (FR1 to FR4, respectively) and 3 complementarity determining regions (CDR1 to CDR3 respectively), in which: CDR1 (AbM numbering) has an amino acid sequence selected from: a) the amino acid sequence of SEQ ID NO: GFTFRSFGMS (SEQ ID NO: 467); b) amino acid sequences that have at least 80% amino acid identity with the amino acid sequence of SEQ ID NO: GFTFRSFGMS (SEQ ID NO: 467); and c) amino acid sequences that have 3, 2, or 1 amino acid difference with the amino acid sequences of SEQ ID NO: GFTFRSFGMS (SEQ ID NO: 467); and CDR2 (AbM numbering) has an amino acid sequence selected from: d) the amino acid sequence of SEQ ID NO: SISGSGSDTL (SEQ ID NO: 468); e) amino acid sequences that have at least 80% amino acid identity with the amino acid sequence of SEQ ID NO: SISGSGSDTL (SEQ ID NO: 468); f) amino acid sequences that have 3, 2, or 1 amino acid difference with the amino acid sequence of SEQ ID NO: SISGSGSDTL (SEQ ID NO: 468); and CDR3 (AbM numbering) has an amino acid sequence selected from: g) the amino acid sequence of SEQ ID NOs: GGSLSRS (SEQ ID NO: 469); h) amino acid sequences that have at least 80% amino acid identity with the amino acid sequence of SEQ ID NO: GGSLSRS (SEQ ID NO: 469); i) amino acid sequences that have 3, 2, or 1 amino acid difference with the amino acid sequence of SEQ ID NO: GGSLSRS (SEQ ID NO: 469).

In certain embodiments, CDR1 consists of the amino acid sequence of SEQ ID NO: GFTFRSFGMS (SEQ ID NO: 467), CDR2 consists of the amino acid sequence of SEQ ID NO: SISGSGSDTL (SEQ ID NO: 468), and CDR3 consists of the amino acid sequence of SEQ ID NO: GGSLSRS (SEQ ID NO: 469).

In certain embodiments, said ISVD that specifically binds human serum albumin is selected from the group consisting of ALB8 (SEQ ID NO: 77), ALB23 (SEQ ID NO: 78), and ALB23002 (SEQ ID NO: 90).

In certain embodiments, said linker is chosen from the group consisting of SEQ ID NOs: 1-8, 56-58, 64-73.

In certain embodiments, the polypeptide or construct further comprises a C-terminal extension.

In certain embodiments, said C-terminal extension is a C-terminal extension (X)ₙ, in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).

In one aspect, the disclosure provides a nucleic acid that encodes an ISVD described herein, or a polypeptide described herein.

In certain embodiments, the nucleic acid is in the form of a genetic construct.

In one aspect, the disclosure provides a non-human host or host cell that expresses, or that under suitable circumstances is capable of expressing, an ISVD described herein, or a polypeptide described herein; and/or that comprises the nucleic acid described herein.

In one aspect, the disclosure provides a method for producing an ISVD described herein, or a polypeptide described herein, at least comprising the steps of: a) expressing, in a suitable (non-human) host cell or host organism or in another suitable expression system, a nucleic acid described herein; optionally followed by: b) isolating and/or purifying the ISVD described herein, or the polypeptide described herein.

In one aspect, the disclosure provides a method for producing an ISVD described herein, or a polypeptide described herein, said method at least comprising the steps of: a) cultivating and/or maintaining a non-human host or host cell described herein under conditions that are such that said non-human host or host cell expresses and/or produces at least one ISVD described herein, or at least one polypeptide described herein; optionally followed by: b) isolating and/or purifying the ISVD a described herein, or polypeptide described herein.

In one aspect, the disclosure provides a composition comprising at least one ISVD described herein, at least one polypeptide or construct described herein, or at least one nucleic acid described herein.

In certain embodiments, the composition described herein is a pharmaceutical composition.

In certain embodiments, the composition described herein is a pharmaceutical composition, that further comprises at least one pharmaceutically acceptable carrier, diluent or excipient and/or adjuvant, and that optionally comprises one or more further pharmaceutically active polypeptides and/or compounds.

In certain embodiments, the ISVD described herein, the polypeptide or construct described herein, or the composition described herein is for use as a medicament.

In certain embodiments, the ISVD described herein, the polypeptide or construct described herein, or the composition described herein, is for use in the diagnosis, prevention and/or treatment of at least one disease and/or disorder.

In certain embodiments, the ISVD described herein, the polypeptide or construct described herein, or the composition described herein, is for use in the diagnosis, prevention and/or treatment of at least one disease and/or disorder that is associated with IL2R beta and/or IL2R-gamma, with its biological or pharmacological activity, and/or with the biological pathways or signalling in which IL2R beta and/or IL2R-gamma is involved.

In certain embodiments, the ISVD according described herein, the polypeptide or construct described herein, or the composition described herein, is for use in the diagnosis, prevention and/or treatment of a disease or disorder.

In one aspect, the disclosure provides a method for the diagnosis, prevention and/or treatment of at least one disease and/or disorder, comprising the administration, to a subject in need thereof, of a pharmaceutically active amount of an ISVD described herein, a polypeptide or construct described herein, or a composition described herein.

In certain embodiments, the method described herein is for the diagnosis, prevention and/or treatment of at least one disease or disorder that is associated with IL2R beta and/or IL2R-gamma, with its biological or pharmacological activity, and/or with the biological pathways or signalling in which IL2R beta and/or IL2R-gamma is involved, said method comprising administering, to a subject in need thereof, a pharmaceutically active amount of at least one ISVD described herein, a polypeptide or construct described herein, or composition described herein.

In certain embodiments, the method described herein is for the diagnosis, prevention and/or treatment of cancer, said method comprising administering, to a subject in need thereof, a pharmaceutically active amount of at least one ISVD described herein, a polypeptide or construct described herein, or a composition described herein.

### BRIEF DESCRIPTION OF THE DRAWINGS

The foregoing and other features and advantages of the present application will be more fully understood from the following detailed description of illustrative aspects taken in conjunction with the accompanying drawings.

FIG. 1 is a schematic drawing of steps in IL-2Rβγ-mediated internalization and endocytic trafficking routes for a multispecific binding protein of the disclosure (e.g., PD-1/IL-2Rβγ bispecific ISVD construct), and a target protein (e.g., a PD-1). One skilled in the art will appreciate that the other multispecific binding protein formats (e.g., an antibody or an immunoadhesin molecule), other target proteins, and additional endocytic trafficking steps are possible.

FIG. 2 is a schematic of an exemplary PD-1/IL-2Rβγ ISVD construct of the disclosure.

FIG. 3 is a schematic of the presence of either the IL-2Rβ and the IL-2Rγ subunits in different compositions to form the receptors for the six members of the IL-2Rγ subunit family (IL-2, IL-4, IL-7, IL-9, IL-15, and IL-21). Notably, both IL-2Rβ and the IL-2Rγ subunits can be present for IL-2 and IL-15 binding. The subsequent signaling cascades impacting different T cell outcomes are also summarized. FIG. 3 is a graphic from Front. Immunol. (2019), vol (10) 263: 1-18.

FIG. 4 are flow cytometry plots gating on the population of PD-1 positive cells after incubation of HEK-IL2 cells for 24-hours either with controls or with 125 nM of ISVD construct 1, ISVD construct 2, ISVD construct 3, or ISVD construct 4.

FIG. 5 is a gel image of a western blot demonstrating the cellular degradation of PD-1 by an exemplary PD-1/IL-2Rβγ ISVD construct described herein. A Human Embryonic Kidney cell line was artificially transfected with a human IL-2 (i.e., HEK-IL2 cells). HEK-IL2 cells expressing PD-1 were incubated with 125 nM of either one of four PD-1/IL-2Rβγ bispecific ISVD constructs (gel lanes 3-4; ISVD construct 1, ISVD construct 2), a PD-1 ISVD construct control (lane 2) or left untreated (lane 1) for 24-hours followed by a wash. Cell lysates were probed with an anti-PD-1 antibody. The level of PD-1 in the cell lysate samples was quantified and normalized to GAPDH control displayed under each sample.

FIG. 6 are flow cytometry plots gating on the population of PD-1 positive cells during a 72-hour time-course experiment after incubation of ISVD construct 1 with HEK-IL2 cells at 0, 2, 24, 28, and 72-hour intervals.

FIG. 7 shows flow cytometry plots displaying the degradation of PD-1 after 24-hours of incubation with four different PD-1/IL-2Rβγ ISVD construct s titrated at increasing concentrations (0-5 nM) in activated T cells analyzed by FACs.

FIGS. 8A-8B show a graph of dose dependent STAT5 activation in Ba/F3 human (FIG. 8A) or cynomolgus (FIG. 8B) IL-2Rβγ pSTAT5 reporter assay after incubation with escalating concentrations of ISVD constructs or a reference molecule. The luminescence is plotted against the concentration of a multivalent ISVD construct or a reference molecule.

FIG. 9 shows a graph of dose dependent STAT5 activation in Ba/F3 human IL-21R STAT5 reporter assay after incubation with escalating concentrations of ISVD constructs or a reference molecule. The luminescence is plotted against the concentration of multivalent ISVD construct or reference molecule.

### DETAILED DESCRIPTION

The present disclosure is directed to, inter alia, a method for degrading a target protein, comprising: contacting an IL-2Rβγ positive cell with a multispecific binding protein, wherein the multispecific binding protein comprises: a) a first cell surface binding moiety that specifically binds to a CD122 (IL-2Rβ) subunit and/or a CD132 (IL-2Rγ) subunit, on the surface of the IL-2Rβγ positive cell, wherein the first cell surface binding moiety comprises at least one immunoglobulin domain or a fragment thereof; and b) a second binding moiety that is operatively linked to the first cell surface binding moiety and that specifically binds to the target protein, wherein specific binding of the multispecific binding protein to the IL-2Rβ subunit and/or the IL-2Rγ subunit facilitates the internalization of the target protein into the IL-2Rβγ positive cell.

### Definitions

It is to be understood that the methods described in this disclosure are not limited to particular methods and experimental conditions such that methods and conditions can vary. It is also to be understood that the terminology used herein is for the purpose of describing particular embodiments only and is not intended to be limiting.

The experiments described herein, unless otherwise indicated, use conventional molecular and cellular biological and immunological techniques within the skill of the art. Such techniques are well known to the skilled worker and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2^{nd} edition). Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. Any single term, single element, single phrase, group of terms, group of phrases, or group of elements described herein can each be specifically excluded from the claims.

Generally, nomenclature used in connection with cell culture, molecular biology, immunology, microbiology, genetics, protein biology, and chemistry described herein is well-known and commonly used in the art. The nomenclatures used in connection with, and the laboratory procedures and techniques of, analytical chemistry, synthetic organic chemistry, and medicinal and pharmaceutical chemistry described herein are those well-known and commonly used in the art.

For the disclosure to be more readily understood, select terms are defined below.

### Polypeptide and Isolated Polypeptide

The term “polypeptide” refers to any polymeric chain of amino acids and encompasses native or artificial proteins, polypeptide analogs or variants of a protein sequence, or fragments thereof, unless otherwise contradicted by context. A polypeptide can be monomeric or polymeric. For a polypeptide (e.g., a polypeptide encoding a first cell surface binding moiety that specifically binds to an IL-2Rβ subunit, an IL-2Rγ subunit, or both IL-2Rβγ subunits on the surface of an IL-2Rβγ positive cell and/or a second binding moiety that is operatively linked to the first cell surface binding moiety and that specifically binds to a target protein), a fragment of a polypeptide optionally contains at least one contiguous or nonlinear paratope of a polypeptide. A fragment polypeptide can be about 25, 50, 75, 100, 150, 200, 250, 300, 350, 400 or more amino acids in length while retaining the capacity to bind to an IL-2Rβ subunit, an IL-2Rγ subunit, or both IL-2Rβγ subunits and a target protein. The precise boundaries of the at least one paratope or paratope fragment can be confirmed using ordinary skill in the art. A polypeptide fragment comprises at least about 5 contiguous amino acids, at least about 10 contiguous amino acids, at least about 15 contiguous amino acids, or at least about 20 contiguous amino acids, at least about 50 contiguous amino acids, at least about 100 contiguous amino acids, at least about 150 contiguous amino acids, at least about 200 contiguous amino acids, at least about 250 contiguous amino acids, at least about 300 contiguous amino acids, at least about 400 contiguous amino acids for example.

In certain aspects, a first cell surface binding moiety polypeptide and a second binding moiety polypeptide are “isolated polypeptides.” The term “isolated polypeptide” refers to a protein or polypeptide that by virtue of its origin or source of derivation is not associated with naturally associated components that accompany it in its native state; is substantially free of other proteins from the same species. An isolated recombinant polypeptide is expressed by a cell from a different species. In some aspects, an isolated polypeptide does not occur in nature. A protein or polypeptide that is chemically synthesized or synthesized in a cellular system can be different from the cell from which it naturally originates and therefore will be “isolated” from its naturally associated components. A protein or polypeptide can also be rendered substantially free of naturally associated components by isolation using protein purification techniques.

### Binding Protein or Binding Polypeptide

As used herein, the term “binding protein,” “binding polypeptide,” or “multispecific binding polypeptide or protein” refer to a protein or polypeptide (e.g., an antibody or specific binding fragment thereof) that contains at least one binding site which is responsible for selectively binding to a target antigen of interest (e.g., a human antigen). Exemplary binding sites include an antibody variable domain, a ligand binding site of a receptor, or a receptor binding site of a ligand. In certain aspects, the binding proteins or binding polypeptides comprise multiple (e.g., two, three, four, or more) binding sites. In certain aspects, the binding protein or binding polypeptide is not a therapeutic enzyme.

Native Residue Vs. Modified Binding Polypeptide

As used herein, the term “native residue” refers to an amino acid residue that occurs naturally at a particular amino acid position of a binding polypeptide (e.g., an antibody or fragment thereof) and which has not been modified, introduced, or altered by the hand of man. As used herein, the term “altered binding protein,” “altered binding polypeptide,” “modified binding protein” or “modified binding polypeptide” shall refer to binding polypeptides and/or binding proteins (e.g., an antibody or fragment thereof) comprising at least one amino acid substitution, deletion and/or addition relative to the native (i.e., wild-type) amino acid sequence, and/or a mutation that results in altered glycosylation (e.g., hyperglycosylation, hypoglycosylation and/or aglycosylation) at one or more amino acid positions relative to the native (i.e., wild-type) amino acid sequence.

### Ligand and Antigen

The term “ligand” refers to any substance capable of binding, or of being bound, to another substance. The term “antigen” or “target antigen” as used herein refers to a molecule or a portion of a molecule that is capable of being bound by the binding site of a binding polypeptide e.g., any substance to which an antibody can be generated. A target antigen may have one or more epitopes.

Although “antigen” is commonly used in reference to an antibody binding substrate, and “ligand” is often used when referring to receptor binding substrates, these terms are not distinguishing, one from the other, and encompass a wide range of overlapping chemical entities. For the avoidance of doubt, antigen and ligand are used interchangeably throughout herein.

Examples of antigens/ligands can be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and any combination thereof.

### Immunoglobulin Domain

The term immunoglobulin domain as used herein can refer to an immunoglobulin A, an immunoglobulin D, an immunoglobulin E, an immunoglobulin G, or an immunoglobulin M. The immunoglobulin domain can be an immunoglobulin heavy chain region or fragment thereof. In some instances, the immunoglobulin domain is from an antibody (e.g., a mammalian antibody, a recombinant antibody, a chimeric antibody, an engineered antibody, a human antibody, a humanized antibody) or an antigen binding fragment thereof. As used herein, the term “antibody” refers to such assemblies (e.g., intact antibody molecules, antibody fragments, or variants thereof) which have significant known specific immunoreactive activity to an antigen of interest (e.g., an IL-2, IL-2R, or IL-2Rβγ associated antigen or a target protein associated antigen). Antibodies and immunoglobulins comprise light and heavy chains, with or without an interchain covalent linkage between them. Basic immunoglobulin structures in vertebrate systems are relatively well understood.

As will be discussed in more detail below, the generic term “antibody” comprises five distinct classes of antibody that can be distinguished biochemically. While all five classes of antibodies are clearly within the scope of the current disclosure, the following discussion will generally be directed to the IgG class of immunoglobulin molecules. With regard to IgG, immunoglobulins comprise two identical light chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Light chains of immunoglobulin are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin isotype subclasses (e.g., IgG1, IgG2, IgG3, IgG4, IgA1, etc.) confer functional specialization. Modified versions of each of these classes and isotypes are readily discernable to the skilled artisan in view of the instant disclosure and, accordingly, are within the scope of the current disclosure.

Both the light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarity determining regions” or “CDRs” interspersed among “framework regions” or “FRs,” as defined herein.

### Constant and Variable Domains

The regions of an immunoglobulin heavy or light chain can be defined as “constant” (C) region or “variable” (V) regions, based on the relative lack of sequence variation within the regions of various class members in the case of a “constant region”, or the significant variation within the regions of various class members in the case of a “variable regions.” The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like. The subunit structures and three-dimensional configurations of the constant regions of the various immunoglobulin classes are well known.

The constant and variable regions of immunoglobulin heavy and light chains are folded into domains. The term “domain” refers to a globular region of a heavy or light chain comprising peptide loops (e.g., comprising 3 to 4 peptide loops) stabilized, for example, by β-pleated sheet and/or intrachain disulfide bond. Constant region domains on the light chain of an immunoglobulin are referred to interchangeably as “light chain constant region domains”, “CL regions” or “CL domains.” Constant domains on the heavy chain (e.g., hinge, CH1, CH2 or CH3 domains) are referred to interchangeably as “heavy chain constant region domains”, “CH” region domains or “CH domains”. Variable domains on the light chain are referred to interchangeably as “light chain variable region domains”, “V_{L} region domains or “V_{L} domains.” Variable domains on the heavy chain are referred to interchangeably as “heavy chain variable region domains”, “V_{H} region domains” or “V_{H} domains.”

By convention the numbering of the variable constant region domains increases as they become more distal from the antigen binding site or amino-terminus of the immunoglobulin or antibody. The N-terminus of each heavy and light immunoglobulin chain is a variable region and at the C-terminus is a constant region; the CH3 and CL domains actually comprise the carboxy-terminus of the heavy and light chain, respectively. Accordingly, the domains of a light chain immunoglobulin are arranged in a V_{L}-CL orientation, while the domains of the heavy chain are arranged in the V_{H}-CH1-hinge-CH2-CH3 orientation.

In some aspects, a IL-2Rβγ positive cell can be contacted with a multispecific binding protein, wherein the multispecific binding protein comprises: a) a first cell surface binding moiety that specifically binds to a CD122 (IL-2Rβ) subunit and/or a CD132 (IL-2Rγ) subunit, on the surface of the IL-2Rβγ positive cell, wherein the first cell surface binding moiety comprises at least one immunoglobulin domain or a fragment thereof; and b) a second binding moiety that is operatively linked to the first cell surface binding moiety and that specifically binds to the target protein, wherein specific binding of the multispecific binding protein to the IL-2Rβ subunit and the IL-2Rγ subunit facilitates the internalization of the target protein into the IL-2Rβγ positive cell.

In some aspects, a multispecific binding protein comprising a first cell surface binding moiety binds to an IL-2Rβ subunit, an IL-2Rγ subunit, or both IL-2Rβγ subunits on the surface of the IL-2Rβγ positive cell, or binds an extracellular domain of the IL-2Rβ subunit, the IL-2Rγ subunit, or both IL-2Rβγ subunits. In some aspects, a first binding moiety that specifically binds to IL-2Rβ, IL-2Rγ, or IL-2Rβγ is an IL-2Rβ, or an IL-2Rγ, or an IL-2Rβγ antibody or an antigen binding fragment thereof. In some aspects, a variable domain(s) specific for IL-2Rβ, IL-2Rγ, or IL-2Rβγ is operatively linked to a first Fc domain polypeptide. In some aspects, a second binding moiety that binds to the target protein comprises an antibody or an antigen binding fragment thereof. In some aspects, the antibody or the antigen binding fragment thereof specific for a target protein comprises a target protein specific variable domain. In some aspects, a target protein specific variable domain for a target protein is operatively linked to a second Fc domain polypeptide. In some aspects, the second binding moiety that specifically binds to the target protein comprises an antibody or an antigen binding fragment thereof.

V_{H} Domain and V_{L} Domain

As used herein, the term “V_{H} domain” includes the amino terminal variable domain of an immunoglobulin heavy chain, and the term “V_{L} domain” includes the amino terminal variable domain of an immunoglobulin light chain.

As used herein, the term “CH1 domain” includes the first (most amino terminal) constant region domain of an immunoglobulin heavy chain that extends, e.g., from about positions 114-223 in the Kabat numbering system (EU positions 118-215). The CH1 domain is adjacent to the V_{H} domain and amino terminal to the hinge region of an immunoglobulin heavy chain molecule and does not form a part of the Fc region of an immunoglobulin heavy chain.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998, 161:4083).

### CH2 Domain

As used herein, the term “CH2 domain” includes the portion of a heavy chain immunoglobulin molecule that extends, e.g., from about positions 244-360 in the Kabat numbering system (EU positions 231-340). The CH2 domain is unique in that it is not closely paired with another domain. Rather, two N-linked branched carbohydrate chains are interposed between the two CH2 domains of an intact native IgG molecule. In certain aspects, a multispecific binding protein of the current disclosure comprises a CH2 domain derived from an IgG1 molecule (e.g., a human IgG1 molecule).

### CH3 Domain

As used herein, the term “CH3 domain” includes the portion of a heavy chain immunoglobulin molecule that extends approximately 110 residues from N-terminus of the CH2 domain, e.g., from about positions 361-476 of the Kabat numbering system (EU positions 341-445). The CH3 domain typically forms the C-terminal portion of the antibody. In some immunoglobulins, however, additional domains may extend from CH3 domain to form the C-terminal portion of the molecule (e.g., the CH4 domain in the μ chain of IgM and the ε chain of IgE). In certain aspects, a multispecific binding protein of the current disclosure comprises a CH3 domain derived from an IgG1 molecule (e.g., a human IgG1 molecule).

### CL Domain

As used herein, the term “CL domain” includes the constant region domain of an immunoglobulin light chain that extends, e.g., from about Kabat position 107A-216. The CL domain is adjacent to the V_{L} domain. In certain aspects, a multispecific binding protein of the current disclosure comprises a CL domain derived from a kappa light chain (e.g., a human kappa light chain).

As indicated above, the variable regions of an antibody allow it to selectively recognize and specifically bind epitopes on antigens. That is, the V_{L} domain and V_{H} domain of an antibody combine to form the variable region (Fv) that defines a three dimensional antigen binding site. This quaternary antibody structure forms the antigen binding site present at the end of each arm of the Y. More specifically, the antigen binding site is defined by three complementary determining regions (CDRs) on each of the heavy and light chain variable regions. As used herein, the term “antigen binding site” includes a site that specifically binds (immunoreacts with) an antigen (e.g., a cell surface or soluble antigen). The antigen binding site includes an immunoglobulin heavy chain and light chain variable region and the binding site formed by these variable regions determines the specificity of the antibody. An antigen binding site is formed by variable regions that vary from one antibody to another. The altered antibodies of the current disclosure comprise at least one antigen binding site.

In certain aspects, a multispecific binding protein of the current disclosure comprise at least two different antigen binding domains that provide for the association of the binding polypeptide with the selected antigen or target. In some aspects, a first cell surface binding moiety binds to IL-2Rβγ on the surface of an IL-2Rβγ positive cell and a second binding moiety binds to a target protein and is linked to the first binding moiety.

In certain aspects, the antigen binding domains are not derived from the same immunoglobulin molecule. In this regard, the variable region may be derived from any type of animal that can be induced to mount a humoral response and generate immunoglobulins against the desired antigen. As such, the variable region of a multispecific binding protein may be, for example, of mammalian origin e.g., may be human, murine, rat, goat, sheep, non-human primate (such as cynomolgus monkeys, macaques, etc.), lupine, or camelid (e.g., from camels, llamas and related species).

In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to the immunoreactive antigen epitope.

### CDR and FR

As used herein, the term “complementarity determining region” or “CDR” refers to sequences of amino acids within antibody variable regions, which confer antigen specificity and binding affinity. In general, there are three CDRs in each heavy chain variable region (HCDR1, HCDR2, HCDR3) and three CDRs in each light chain variable region (LCDR1, LCDR2, LCDR3). “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (AHo numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

A “CDR” or “complementarity determining region,” or individual specified CDRs (e.g., “HCDR1,” “HCDR2,” “HCDR3”), of a given antibody or region thereof, such as a variable region thereof, should be understood to encompass a (or the specific) complementarity determining region as defined by any of the known schemes. Likewise, an “FR” or “framework region,” or individual specified FRs (e.g., “FR-H1,” “FR-H2”) of a given antibody or region thereof, such as a variable region thereof, should be understood to encompass a (or the specific) framework region as defined by any of the known schemes. In some instances, the scheme for identification of a particular CDR or FR is specified, such as the CDR as defined by the IMGT, Kabat, Chothia, AbM, or Contact method. In other cases, the particular amino acid sequence of a CDR or FR is given. However, alternative CDRs defined by other schemes are also encompassed by the present disclosure, such as those determined by abYsis Key Annotation (Website: abysis.org/abysis/sequence_input/key_annotation/key_annotation.cgi).

In the present application, unless indicated otherwise, CDR sequences of immunoglobulin single variable domains (ISVDs) were determined according to the AbM numbering as described in Kontermann and Dubel (Eds. 2010, Antibody Engineering, vol 2, Springer Verlag Heidelberg Berlin, Martin, Chapter 3, pp. 33-51). According to this method, FR1 of an ISVD comprises the amino acid residues at positions 1-25, CDR1 of an ISVD comprises the amino acid residues at positions 26-35, FR2 of an ISVD comprises the amino acids at positions 36-49, CDR2 of an ISVD comprises the amino acid residues at positions 50-58, FR3 of an ISVD comprises the amino acid residues at positions 59-94, CDR3 of an ISVD comprises the amino acid residues at positions 95-102, and FR4 of an ISVD comprises the amino acid residues at positions 103-113.

Determination of CDR regions may also be done according to different methods, such as according to Kabat definition, Chothia definition, or IMGT definition (see Kontermann and Dubel, Eds. 2010, Antibody Engineering, vol 2, Springer Verlag Heidelberg Berlin, Martin, Chapter 3, pp. 33-51). According to the Kabat CDR definition, FR1 of an ISVD comprises the amino acid residues at positions 1-30, CDR1 of an ISVD comprises the amino acid residues at positions 31-35, FR2 of an ISVD comprises the amino acids at positions 36-49, CDR2 of an ISVD comprises the amino acid residues at positions 50-65, FR3 of an ISVD comprises the amino acid residues at positions 66-94, CDR3 of an ISVD comprises the amino acid residues at positions 95-102, and FR4 of an ISVD comprises the amino acid residues at positions 103-113.

### Antibody Variant

Exemplary multispecific binding proteins can comprise antibody variants and/or specific antibody binding fragments variants. As used herein, the term “antibody variant” and “antibody specific binding fragment variant” includes synthetic and engineered forms of antibodies which are altered such that they are not naturally occurring, e.g., antibodies that comprise at least two heavy chain portions but not two complete heavy chains (such as, domain deleted antibodies or minibodies) and multispecific forms of antibodies altered to bind to two or more different antigens or to different epitopes on a single antigen); heavy chain molecules joined to scFv molecules and the like.

### Antigen Binding Fragments

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen binding fragments thereof. Other engineered molecules, such as domain specific binding proteins, single domain binding proteins, domain deleted binding proteins, chimeric binding proteins, CDR grafted binding proteins, diabodies, triabodies, tetrabodies, minibodies, immunoglobulin single variable domains (ISVDs) (e.g., monovalent ISVDs, bivalent ISVDs, etc.), small modular immunopharmaceuticals (SMIPs), and shark variable IgNAR domains, are also encompassed within the expression “antigen binding fragment,” as used herein. The term “multispecific antibody” denotes a binding fragment or derivative thereof that combines the antigen-binding sites of two or more antibodies within a single molecule. The terms “antigen binding portion”, “antigen binding fragment”, “binding protein” or “binding moiety” and the like, as used herein, include any naturally occurring, enzymatically obtainable, synthetic, or genetically engineered polypeptide or glycoprotein that specifically binds to at least one target antigen to form a complex.

In certain aspects, a binding moiety can refer to one or more fragments of an IL-2Rβ subunit, an IL-2Rγ subunit, or both IL-2Rβγ subunits multispecific binding protein that retain the ability to specifically bind to an IL-2Rβ subunit, an IL-2Rγ subunit, or both IL-2Rβγ subunits on the surface of an IL-2Rβγ positive cell and/or a second target protein or a target protein.

In certain aspects, the term “antigen binding fragment” refers to a polypeptide fragment of a multispecific binding protein. Antigen binding fragments of a multispecific binding protein, or a binding fragment or derivative thereof can be derived, e.g., from full multispecific binding protein molecules using any suitable standard techniques such as proteolytic digestion or recombinant genetic engineering techniques involving the manipulation and expression of DNA encoding multispecific binding protein, or a binding fragment or derivative thereof variable and (optionally) constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add, or delete amino acids, etc.

### Multispecific

As used herein, a “multispecific” binding protein is a binding protein that specifically binds two or more types of antigens.

A multispecific binding protein that binds two antigens, and/or two different epitopes of different antigens, is also referred to herein as a “bispecific” binding protein (e.g., a multispecific binding protein that binds an IL-2Rβ subunit epitope or an IL-2Rγ subunit epitope and the target protein of interest).

A multispecific binding protein that binds three antigens, and/or three different epitopes, is also referred to herein as a “trispecific” binding protein (e.g., multispecific binding protein that binds both an IL-2Rβ subunit epitope and an IL-2Rγ subunit epitope and the target protein of interest).

Thus, the multispecific binding protein is able to bind two or more different targets simultaneously, for example, an IL-2Rβ subunit and/or an IL-2Rγ subunit and a target protein of interest. Genetic engineering can be used to design, modify, and produce the multispecific binding protein, or a binding fragment or derivative thereof with a desired set of binding properties and effector functions.

In some aspects, a multispecific binding protein of the disclosure comprises a first cell surface binding moiety that binds to an IL-2Rβ subunit epitope. In some aspects, a multispecific binding protein of the disclosure comprises a first cell surface binding moiety that binds to an IL-2Rγ subunit epitope.

In some aspects, a multispecific binding protein of the disclosure comprises a first cell surface binding moiety that binds to both an IL-2Rβ epitope and an IL-2Rγ subunit epitope. In some aspects, the IL-2Rβ epitope and the IL-2Rγ epitope are different epitopes. In some aspects, the IL-2Rβ epitope and the IL-2Rγ epitope are the same epitope.

An IL-2Rβ, IL-2Rγ, or IL-2Rβγ targeting multispecific binding protein of this disclosure, in some aspects, comprises a first cell surface binding moiety that specifically binds to an IL-2Rβ subunit, an IL-2Rγ subunit, or both IL-2Rβγ subunits on the surface of an IL-2Rβγ positive cell and a second binding moiety that is operatively linked to the first cell surface binding moiety and specifically binds to a target protein. In some aspects, a first cell surface binding moiety and a second binding moiety of the multispecific binding moiety are each independently selected from a group consisting of an antibody or an antigen-binding fragment thereof such as, a V_{H}, a Fab, a Fab′, a F(ab′)2, a variable fragment (Fv), a disulfide-stabilized Fv (dsFv), a single-chain Fv (scFv), a tandem di-scFv, a tandem tri-scFv, a minibody, a diabody, a triabody, a tetrabody, an immunoglobulin single variable domain (ISVD), such as, a V_{HH} (including humanized V_{HH}), a camelized V_{H}, a single domain antibody, a domain antibody, or a dAb, or an AFFIBODY®.

In some aspects, a multispecific binding protein of the disclosure comprises a V_{H}, a Fab, a Fab′, a F(ab′)2, a variable fragment (Fv), a disulfide-stabilized Fv (dsFv), a single-chain Fv (scFv), a diabody, a triabody, an immunoglobulin single variable domain (ISVD), or an AFFIBODY®. In some aspects, the multispecific binding protein further comprises a Fc domain or a variant thereof. In one aspect, the ISVD is a V_{HH}, humanized V_{HH}, a camelized V_{H}, a domain antibody (dAb) or a VNAR. In some aspects, the scFV is a linear scFV or a tandem scFV.

Techniques for making multispecific binding proteins (e.g., multispecific antibodies) include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al., EMBO J. 10 (1991) 3655-3659), and “knob-in-hole” engineering (see, e.g., U.S. Pat. No. 5,731,168). In some embodiments, the bispecific antibodies are prepared using a macro-assembly technique using two half-antibodies, e.g., as described in Spiess, C., Merchant, M., Huang, A. et al. Bispecific antibodies with natural architecture produced by co-culture of bacteria expressing two distinct half-antibodies. Nat Biotechnol 31, 753-758 (2013).

Multispecific antibodies may also be made by engineering electrostatic steering effects for making binding protein Fc-heterodimeric molecules (WO 2009/089004); cross-linking two or more antibodies or fragments (see, e.g., U.S. Pat. No. 4,676,980, and Brennan, M. et al., Science 229 (1985) 81-83); using leucine zippers to produce bi-specific antibodies (see, e.g., Kostelny, S. A. et al., J. Immunol. 148 (1992) 1547-1553); using “diabody” technology for making multispecific binding protein fragments (see, e.g., Holliger, P. et al., Proc. Natl. Acad. Sci. USA 90 (1993) 6444-6448); and using single-chain Fv (scFv) dimers (see, e.g., Gruber, M et al., J. Immunol. 152 (1994) 5368-5374); and preparing trispecific binding proteins as described (see e.g., in Tutt, A. et al., J. Immunol. 147 (1991) 60-69).

A wide variety of recombinant multispecific binding protein formats have been developed, e.g., by fusion an IgG binding protein format and single chain domains (see Kontermann R E, mAbs 4:2, (2012) 1-16). Multispecific binding proteins wherein the variable domains V_{L} and V_{H} or the constant domains CL and CH1 are replaced by each other are described in WO2009080251 and WO2009080252.

In one aspect, a first and a second IgG Fc domain polypeptide of a multispecific binding protein can dimerize. In some aspects, a multispecific binding protein of the disclosure dimerizes by knobs-into-holes interactions, Fab arm exchange (FAE), electrostatic steering interactions, hydrophobic interactions, or any combination thereof.

### Multispecific Binding Protein

### Binding Moiety

The term “binding moiety” is used herein in the broadest sense to encompass any chemical entity capable of specific binding to a target, such as an antigen (e.g., IL-2Rβγ). The terms “binding moiety” and “binding domain” can be used interchangeably herein.

In some aspects, a method for degrading a target protein, comprises: contacting an IL-2Rβγ positive cell with a multispecific binding protein, wherein the multispecific binding protein comprises: a) a first cell surface binding moiety that specifically binds to a CD122 (IL-2Rβ) subunit and/or a CD132 (IL-2Rγ) subunit, on the surface of the IL-2Rβγ positive cell, wherein the first cell surface binding moiety comprises at least one immunoglobulin domain or a fragment thereof; and b) a second binding moiety that is operatively linked to the first cell surface binding moiety and that specifically binds to the target protein, wherein specific binding of the multispecific binding protein to the IL-2Rβ subunit and/or the IL-2Rγ subunit facilitates the internalization of the target protein into the IL-2Rβγ positive cell.

Examples of a binding moiety of the multispecific binding protein described herein can comprise an antibody, a V_{H}, a Fab, a Fab′, a F(ab′)2, a variable fragment (Fv), a disulfide-stabilized Fv (dsFv), a single-chain Fv (scFv), a diabody, a triabody, an immunoglobulin single variable domain (ISVD) (including a VHH, such as a humanized VHH, a camelized VH, a domain antibody (dAb) or a VNAR), or an AFFIBODY®. Other binding moiety examples include a Fab fragment, a F(ab′)2 fragment, an Fv fragment a fragment containing a complementarity determining region (CDR), an isolated CDR, or other suitable fragment.

In certain aspects, a first cell surface binding moiety and a second binding moiety of a multispecific binding protein each independently comprise an antigen binding fragment that comprises at least one variable domain of an antibody or an antigen binding fragment. At least one constant domain can optionally be covalently linked to one or both the first and second binding moieties. The variable domain(s) can be of any size or amino acid composition and will generally comprise at least one CDR, which is adjacent to or in frame with one or more framework sequences. In antigen binding fragments having a V_{H} domain associated with a V_{L} domain, the V_{H} and V_{L} domains can be situated relative to one another in any suitable arrangement. For example, the variable region can be dimeric and contain V_{H} V_{H}, V_{H}V_{L} or V_{L} V_{L} dimers. Alternatively, the antigen binding fragment can contain a monomeric V_{H} or V_{L} domain.

Non limiting, exemplary configurations of variable and constant domains that can be found within an antigen binding fragment include: (i) V_{H} CH1; (ii) V_{H} CH2; (iii) V_{H} CH3; (iv) V_{H} CH1 CH2; (v) V_{H} CH1 CH2 CH3; (vi) V_{H} CH2 CH3; (vii) V_{H} CL; (viii) V_{L} CH1; (ix) V_{L} CH2; (X) V_{L} CH3; (xi) V_{L} CH1 CH2; (xii) V_{L} CH1 CH2 CH3; (xiii) V_{L} CH2 CH3; and (xiv) V_{L} CL. In any configuration of variable and constant domains, including any of the exemplary configurations listed above, the variable and constant domains can be either directly linked to one another or can be linked by a full or partial hinge or linker region. A hinge region can comprise of at least 2 (e.g., 5, 10, 15, 20, 40, 60 or more) amino acids, which result in a flexible or semi flexible linkage between adjacent variable and/or constant domains in a single polypeptide molecule. Moreover, an antigen binding fragment of an IL-2Rβ subunit, an IL-2Rγ subunit, or both IL-2Rβγ subunits targeting multispecific binding protein of this disclosure can comprise a homo dimer or hetero dimer (or other multimer) of any of the variable and constant domain configurations listed above in non-covalent association with one another and/or with one or more monomeric V_{H} or V_{L} domain (e.g., by disulfide bond(s)).

### Fab, F(ab′)2, Fab′

The term “Fab” denotes a binding protein or a binding fragment thereof having a molecular weight of about 50,000 Da and antigen binding activity, in which about a half of the N-terminal side of H chain and the entire L chain, among fragments obtained by treating IgG with a protease, papain, are bound together through a disulfide bond.

In certain aspects, an IL-2Rβ subunit, an IL-2Rγ subunit, or both IL-2Rβγ subunits targeting multispecific binding protein comprises a first cell surface binding moiety comprising a Fab specific for an IL-2Rβ subunit, an IL-2Rγ subunit, or both IL-2Rβγ subunits. In certain aspects, an IL-2Rβ subunit, an IL-2Rγ subunit, or both IL-2Rβγ subunits targeting multispecific binding protein comprises a second binding moiety comprising a Fab that specifically binds to the target protein. In certain aspects, the first and the second binding moiety of an IL-2Rβ subunit, an IL-2Rγ subunit, or both IL-2Rβγ subunits targeting multispecific binding protein, is each independently a Fab.

The term F(ab′)2 refers to a binding protein or a binding fragment thereof having a molecular weight of about 100,000 Da and antigen binding activity, which is slightly larger than a Fab bound via a disulfide bond of the hinge region, among fragments obtained by treating IgG with a protease, pepsin.

In certain aspects, an IL-2Rβ subunit, an IL-2Rγ subunit, or both IL-2Rβγ subunits targeting multispecific binding protein comprises a first cell surface binding moiety comprising a F(ab′)2 specific for an IL-2Rβ subunit, an IL-2Rγ subunit, or both IL-2Rβγ subunits. In certain aspects, an IL-2Rβ subunit, an IL-2Rγ subunit, or both IL-2Rβγ subunits targeting multispecific binding protein comprises a second binding moiety comprising a F(ab′)2 that specifically binds to a target protein. In certain aspects, the first and the second binding moiety of an IL-2Rβ subunit, an IL-2Rγ subunit, or both IL-2Rβγ subunits targeting multispecific binding protein, is each independently a F(ab′)2.

The term Fab′ refers to a binding protein or a binding fragment having a molecular weight of about 50,000 Da and antigen binding activity, which is obtained by cutting a disulfide bond of the hinge region of the F(ab′)2.

In certain aspects, an IL-2Rβ subunit, an IL-2Rγ subunit, or both IL-2Rβγ subunits targeting multispecific binding protein comprises a first cell surface binding moiety comprising a Fab′ that specifically binds an IL-2Rβ subunit, an IL-2Rγ subunit, or both IL-2Rβγ subunits. In certain aspects, an IL-2Rβ subunit, an IL-2Rγ subunit, or both IL-2Rβγ subunits targeting multispecific binding protein comprises a second binding moiety comprising a Fab′ that specifically binds to a target protein. In certain aspects, the first and the second binding moiety of an IL-2Rβ subunit, an IL-2Rγ subunit, or both IL-2Rβγ subunits targeting multispecific binding protein is each independently a Fab′.

scFV

A single chain Fv (“scFv”) polypeptide is a covalently linked V_{H}: V_{L} heterodimer that is usually expressed from a gene fusion including V_{H} and V_{L} encoding genes linked by a peptide-encoding linker. A human scFv fragment includes CDRs that are held in appropriate conformation by, e.g., using gene recombination techniques. Divalent and multivalent multi-specific binding proteins, or binding fragments or derivatives thereof can form either spontaneously by association of monovalent scFvs, or can be generated by coupling monovalent scFvs by a peptide linker, such as divalent sc(Fv)2. A “dsFv” is a V_{H}: V_{L} heterodimer stabilized by a disulfide bond. “(dsFv)2” denotes two dsFv coupled by a peptide linker.

In certain aspects, an IL-2Rβ subunit, an IL-2Rγ subunit, or both IL-2Rβγ subunits targeting multispecific binding protein comprises a first cell surface binding moiety comprising a scFv that specifically binds an IL-2Rβ subunit, an IL-2Rγ subunit, or both IL-2Rβγ subunits. In certain aspects, an IL-2Rβ subunit, an IL-2Rγ subunit, or both IL-2Rβγ subunits targeting multispecific binding protein comprises a second binding moiety that comprises a scFv that specifically binds to the target protein. In certain aspects, the first and the second binding moiety of an IL-2Rβ subunit, an IL-2Rγ subunit, or both IL-2Rβγ subunits targeting multispecific binding protein is each independently a scFV. In some aspect, the scFV is a linear scFV or a tandem scFV.

### Immunoglobulin Single Variable Domain

The term “immunoglobulin single variable domain” (ISVD), interchangeably used with “single variable domain”, defines a genus of immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)2, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e., a total of 6 CDRs will be involved in antigen binding site formation. In contrast, ISVDs are capable of specifically binding to an epitope of the antigen without pairing with an additional immunoglobulin variable domain. The binding site of an ISVD is formed by a single V_{H}, a single V_{HH} or single V_{L} domain.

As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy-chain ISVD, such as a VHH, including a humanized VHH, a VH, including a camelized VH and a human VH. In one embodiment, it is a VHH, a camelized VH or humanized VHH.

For example, the immunoglobulin single variable domain may be a single domain antibody (or an amino acid sequence that is suitable for use as a single domain antibody), a “dAb” or dAb (or an amino acid sequence that is suitable for use as a dAb) or a Nanobody® ISVD (as defined herein, and including but not limited to a VHH); other single variable domains, or any suitable fragment of any one thereof.

Camelid heavy-chain antibodies are functional antibodies devoid of light chains and CH1 domains which are naturally produced by the Camelidae species. The antigen recognition site is formed by a single domain called V_{HH}. Sharks and other cartilaginous fish also naturally produce heavy-chain antibodies that recognize antigens with their single domain variable regions called VNAR.

The terms “V_{HH} domains”, “V_{HH}S”, “V_{HH} antibody fragments”, and “V_{HH} antibodies”, are described as the antigen binding immunoglobulin variable domain of “heavy chain antibodies” (i.e., “antibodies devoid of light chains”; see Hamers-Casterman et al. (1993), Nature, vol. 363: 446-448). The term “V_{HH} domain” has been chosen to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}S see Muyldermans (2001), J. Biotechnol., vol. 74(4): 277-302.

In particular, the immunoglobulin single variable domain may be a NANOBODY® ISVD (such as a VHH, including a humanized VHH or camelized VH) or a suitable fragment thereof. [Note: NANOBODY® and NANOBODIES® are registered trademarks of Ablynx N.V.]

A “humanized VHH” comprises an amino acid sequence that corresponds to the amino acid sequence of a naturally occurring VHH domain, but that has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g. indicated above). This can be performed in a manner known per se, which will be clear to the skilled person, for example on the basis of the prior art (e.g., WO 2008/020079). Again, it should be noted that such humanized VHHs can be obtained in any suitable manner known per se and thus are not strictly limited to polypeptides that have been obtained using a polypeptide that comprises a naturally occurring VHH domain as a starting material.

A “camelized VH” comprises an amino acid sequence that corresponds to the amino acid sequence of a naturally occurring VH domain, but that has been “camelized”, i.e. by replacing one or more amino acid residues in the amino acid sequence of a naturally occurring VH domain from a conventional 4-chain antibody by one or more of the amino acid residues that occur at the corresponding position(s) in a VHH domain of a (camelid) heavy chain antibody. This can be performed in a manner known per se, which will be clear to the skilled person, for example on the basis of the description in the prior art (e.g., Davies and Riechman (1994 and 1996), supra). Such “camelizing” substitutions are inserted at amino acid positions that form and/or are present at the VH-VL interface, and/or at the so-called Camelidae hallmark residues, as defined herein (see for example WO 94/04678 and Davies and Riechmann (1994 and 1996), supra). In one embodiment, the VH sequence that is used as a starting material or starting point for generating or designing the camelized VH is a VH sequence from a mammal, such as the VH sequence of a human being, such as a VH3 sequence. However, it should be noted that such camelized VH can be obtained in any suitable manner known per se and thus are not strictly limited to polypeptides that have been obtained using a polypeptide that comprises a naturally occurring VH domain as a starting material.

In certain aspects, an IL-2Rβ subunit, an IL-2Rγ subunit, or both IL-2Rβγ subunits targeting multispecific binding protein comprises a first cell surface binding moiety comprising an ISVD that specifically binds an IL-2Rβ subunit, an IL-2Rγ subunit, or both IL-2Rβγ subunits. In certain aspects, an IL-2Rβ subunit, an IL-2Rγ subunit, or both IL-2Rβγ subunits targeting multispecific binding protein comprises a second binding moiety that comprises an ISVD that specifically binds to the target protein. In certain aspects, the first and the second binding moiety of the multispecific binding protein is each independently an ISVD.

In certain aspects, a multispecific binding protein comprises a first cell surface binding moiety comprises a V_{HH} that specifically binds an IL-2Rβ subunit, an IL-2Rγ subunit, or both IL-2Rβγ subunits. In certain aspects, a multispecific binding protein comprises a second binding moiety comprising a V_{HH} that specifically binds to the target protein. In certain aspects, the first and the second binding moiety of the multispecific binding protein is each independently a V_{HH}. In some aspects, a V_{HH} is humanized V_{HH}.

In certain aspects, a multispecific binding protein comprises a first cell surface binding moiety comprising a VNAR that specifically binds an IL-2Rβ subunit, an IL-2Rγ subunit, or both IL-2Rβγ subunits. In certain aspects, a multispecific binding protein comprises a second binding moiety that comprises a VNAR that specifically binds to the target protein. In certain aspects, the first and the second binding moiety of the multispecific binding protein is each independently a VNAR.

In certain aspects, a multispecific binding protein comprises a first cell surface binding moiety comprising a domain antibody (dAb) or camelized VH that specifically binds an IL-2Rβ subunit, an IL-2Rγ subunit, or both IL-2Rβγ subunits. In certain aspects, an IL-2Rβ subunit, an IL-2Rγ subunit, or both IL-2Rβγ subunits targeting multispecific binding protein comprises a second binding moiety that comprises a domain antibody (dAb) or camelized VH that specifically binds to the target protein. In certain aspects, the first and the second binding moiety of an IL-2Rβ subunit, an IL-2Rγ subunit, or both IL-2Rβγ subunits targeting multispecific binding protein is each independently a domain antibody (dAb) or camelized VH.

In some aspects, the antigen-binding fragments of the disclosure are immunoglobulin single variable domains, such as a domain antibody, a “dAb”, a V_{HH} (including a humanized V_{HH}), a camelized V_{H}, other single variable domains, or any suitable fragment of any one thereof. In particular, antigen-binding fragments of the disclosure may be a V_{HH} or a fragment thereof.

In certain aspects, methods of the disclosure comprise contacting an IL-2Rβγ positive cell with a bispecific ISVD construct, wherein the bispecific ISVD construct comprises: a) a first ISVD that specifically binds to an IL-2Rβ subunit, an IL-2Rγ subunit, or both IL-2Rβγ subunits on the surface of an IL-2Rβγ positive cell; and b) a second ISVD that specifically binds to a target protein of interest, such that the bispecific ISVD construct binds to the membrane-bound IL-2Rβ subunit, IL-2Rγ subunit, or both IL-2Rβγ subunits on the surface of the IL-2Rβγ positive cell and to the target protein.

In certain aspects, methods of the disclosure comprise contacting an IL-2Rβγ positive cell with a bispecific NANOBODY® ISVD (such as a V_{HH}, including a humanized V_{H} or a camelized V_{H}), wherein the bispecific NANOBODY® ISVD comprises: a) a first NANOBODY® ISVD (such as a V_{HH}, including a humanized V_{H} or a camelized V_{H}) that specifically binds to an IL-2Rβ subunit, an IL-2Rγ subunit, or both IL-2Rβγ subunits on the surface of the IL-2Rβγ positive cell; and b) a second NANOBODY® ISVD (such as a V_{HH}, including a humanized V_{H} or a camelized V_{H}) that specifically binds to the target protein of interest, such that the bispecific NANOBODY® ISVD binds to the an IL-2Rβ subunit, an IL-2Rγ subunit, or both IL-2Rβγ subunits on the surface of the IL-2Rβγ positive cell and to the target protein.

ISVDs of the so-called “V_{H}3 class” (i.e., ISVDs with a high degree of sequence homology to human germline sequences of the V_{H}3 class such as DP-47, DP-51 or DP-29), can be used herein. Furthermore, any type of ISVD directed against an IL-2Rβ subunit, an IL-2Rγ subunit, or both IL-2Rβγ subunits on the surface of an IL-2Rβγ positive cell and/or target protein, including for example, ISVDs belonging to the so-called “V_{H}4 class” (i.e., ISVDs with a high degree of sequence homology to human germline sequences of the V_{H}4 class such as DP-78), as for example described in WO 2007/118 670 A1, can be used.

ISVDs (in particular V_{HH} sequences and partially humanized V_{HH}S) can in particular be characterized by the presence of one or more “Hallmark residues” (as described herein in Table 1 and in subsequent paragraphs describing NANOBODY® immunoglobulin single variable domains) such that the ISVD is a NANOBODY® ISVD.

Thus, generally, a NANOBODY® ISVD (in particular a V_{HH}, including (partially or fully) humanized V_{HH} and camelized V_{H}) can be defined as an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively, and in which one or more of the Hallmark residues are as further defined in Table 1.

In particular, a NANOBODY® ISVD (in particular a V_{HH}, including (partially) humanized V_{HH} and camelized V_{H}) can be an amino acid sequence with the (general) structure:

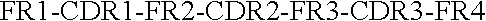

in which FR1 to FR4 refer to framework regions 1 to 4, respectively, and in which CDR1 to CDR3 refer to the complementarity determining regions 1 to 3, respectively, and in which the framework sequences are as further defined herein.

In particular, an ISVD can be an amino acid sequence with the (general) structure:

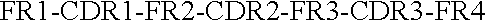

in which FR1 to FR4 refer to framework regions 1 to 4, respectively, and in which CDR1 to CDR3 refer to the complementarity determining regions 1 to 3, respectively.

ISVDs can specifically bind to (as defined herein) and/or are directed against an IL-2Rβ subunit, an IL-2Rγ subunit, or both IL-2Rβγ subunits on the surface of an IL-2Rβγ positive cell and/or the target protein. Also useful are suitable fragments of these ISVDs and polypeptides that comprise, consist, or essentially consist of one or more of such ISVDs and/or suitable fragments of the ISVDs. The term “immunoglobulin single variable domain (ISVD)” encompasses a V_{HH}, a humanized V_{HH} or a camelized V_{H} (such as a camelized human V_{H}) or generally a sequence optimized V_{HH} (such as e.g., optimized for chemical stability and/or solubility, maximum overlap with known human framework regions and maximum expression), as well as domain antibodies such as dAb and sdAbs.

Generally, NANOBODY® immunoglobulin single variable domains (ISVDs) (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a NANOBODY® ISVD can be defined as an immunoglobulin sequence with the (general) structure

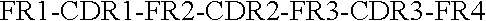

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a NANOBODY® ISVD can be an immunoglobulin sequence with the (general) structure

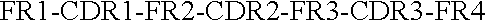

in which FR1 to FR4 refer to framework regions 1 to 4, respectively, and in which CDR1 to CDR3 refer to the complementarity determining regions 1 to 3, respectively, and in which the framework sequences are as further defined herein.

In particular, a NANOBODY® ISVD can be an immunoglobulin sequence with the (general) structure

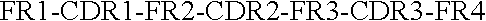

in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table 1 below.

TABLE 1
Hallmark Residues in Nanobody ® ISVDs.

| Position | Human V_{H}3 | Hallmark Residues |
|---|---|---|
| 11 | L, | L, S, V, M, W, F, T, Q, E, A, R, G, K, Y, N, P, I; preferably L |
| | predominantly L | |
| | V; | |
| 37 | V, I, F; usually V | F⁽¹⁾, Y, V, L, A, H, S, I, W, C, N, G, D, T, P, preferably F⁽¹⁾ or |
| | | Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾;most preferably G⁽²⁾ or Q⁽³⁾ |
| 45⁽⁸⁾ | L | L⁽²⁾, R⁽³⁾, P, H, F, G, Q, S, E, T, Y, C, I, D, V; preferably L⁽²⁾ or |
| | | R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, N, |
| | | D; preferably W⁽²⁾, L⁽¹⁾ or F⁽¹⁾ |
| 83 | R or K; | R, K⁽⁵⁾, T, E⁽⁵⁾, Q, N, S, I, V, G, M, L, A, D, Y, H; preferably K |
| | usually R | or R; most preferably K |
| 84 | A, T, D; | P⁽⁵⁾, S, H, L, A, V, I, T, F, D, R, Y, N, Q, G, E; preferably P |
| | predominantly A | |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; | Q, L⁽⁷⁾, R, P, E, K, S, T, M, A, H; preferably Q or L⁽⁷⁾ |
| | predominantly L | |

Notes:
⁽¹⁾In particular, but not exclusively, in combination with KERE (SEQ ID NO: 9) or KQRE (SEQ ID NO: 10) at positions 43-46.
⁽²⁾Usually as GLEW (SEQ ID NO: 11) at positions 44-47.
⁽³⁾Usually as KERE (SEQ ID NO: 9) or KQRE (SEQ ID NO: 10) at positions 43-46, e.g., as KEREL (SEQ ID NO: 12), KEREF (SEQ ID NO: 13), KQREL (SEQ ID NO: 14), KQREF (SEQ ID NO: 15), KEREG (SEQ ID NO: 16), KQREW (SEQ ID NO: 17)or KQREG (SEQ ID NO: 18) at positions 43-47. Alternatively, also sequences such as TERE (SEQ ID NO: 19) (for example TEREL (SEQ ID NO: 20)), TQRE (SEQ ID NO: 21) (for example TQREL (SEQ ID NO: 22)), KECE (SEQ ID NO: 23) (for example KECEL (SEQ ID NO: 24) or KECER (SEQ ID NO: 25)), KQCE (SEQ ID NO: 26) (for example KQCEL (SEQ ID NO: 27)), RERE (SEQ ID NO: 28) (for example REREG (SEQ ID NO: 29)), RQRE (SEQ ID NO: 30) (for example RQREL (SEQ ID NO: 31), RQREF (SEQ ID NO: 32) or RQREW (SEQ ID NO: 33) ), QERE (SEQ ID NO: 34) (for example QEREG (SEQ ID NO: 35)), QQRE (SEQ ID NO: 36), (for example QQREW (SEQ ID NO: 37), QQREL (SEQ ID NO: 38) or QQREF (SEQ ID NO: 39)), KGRE (SEQ ID NO: 40)(for example KGREG (SEQ ID NO: 41)), KDRE (SEQ ID NO: 42) (for example KDREV (SEQ ID NO: 43)) are possible. Some other possible, but less preferred sequences include for example DECKL (SEQ ID NO: 44) and NVCEL (SEQ ID NO: 45).
⁽⁴⁾With both GLEW (SEQ ID NO: 11) at positions 44-47 and KERE (SEQ ID NO: 9) or KQRE (SEQ ID NO: 10) at positions 43-46.
⁽⁵⁾Often as KP or EP at positions 83-84 of naturally occurring VHH domains.
⁽⁶⁾In particular, but not exclusively, in combination with GLEW (SEQ ID NO: 11) at positions 44-47.
⁽⁷⁾With the proviso that when positions 44-47 are GLEW (SEQ ID NO: 11), position 108 is always Q in (non-humanized) VHH sequences that also contain a W at 103.
⁽⁸⁾The GLEW (SEQ ID NO: 11) group also contains GLEW (SEQ ID NO: 11)-like sequences at positions 44-47, such as for example GVEW (SEQ ID NO: 46), EPEW (SEQ ID NO: 47), GLER (SEQ ID NO: 48), DQEW (SEQ ID NO: 49), DLEW (SEQ ID NO: 50), GIEW (SEQ ID NO: 51), ELEW (SEQ ID NO: 52), GPEW (SEQ ID NO: 53), EWLP (SEQ ID NO: 54), GPER (SEQ ID NO: 55), GLER (SEQ ID NO: 48) and ELEW (SEQ ID NO: 52).

The framework sequences are (a suitable combination of) immunoglobulin framework sequences or framework sequences that have been derived from immunoglobulin framework sequences (for example, by humanization or camelization). For example, the framework sequences can be framework sequences derived from a light chain variable domain (e.g., a V_{L}-sequence) and/or from a heavy chain variable domain (e.g., a V_{H}-sequence or V_{HH} sequence). In certain aspects, the framework sequences are either framework sequences that have been derived from a V_{HH}-sequence (in which said framework sequences may optionally have been partially or fully humanized) or are conventional V_{H} sequences that have been camelized.

In certain aspects, the framework sequences present in the ISVD sequences used in the methods described herein may contain one or more of hallmark residues (as defined in Table 1), such that the ISVD sequence is a NANOBODY® ISVD, such as e.g., a V_{HH}, including a humanized V_{HH} or camelized V_{H}.

In certain aspects, a first cell surface binding moiety and a second binding moiety of a multispecific binding protein is each independently an immunoglobulin single variable domain (ISVD). Examples of immunoglobulin single variable domains (ISVDs) include variable domains obtained from heavy chain antibodies (V_{HH}S), variable domains obtained from antibodies naturally devoid of light chains (V_{HH}s), ISVDs derived from conventional four-chain antibodies, engineered ISVDs. ISVDs may be derived from any species including, but not limited to mouse, human, camel, llama, goat, rabbit, bovine. ISVDs may be naturally occurring ISVDs present in a heavy chain antibody devoid of light chains. Camelidae species, for example camel, dromedary, llama, alpaca and guanaco, produce heavy chain antibodies naturally devoid of light chain. Camelid heavy chain antibodies also lack the CH1 domain.

In certain aspects, methods of the disclosure comprise contacting an IL-2Rβγ positive cell with a bispecific ISVD construct, wherein the bispecific ISVD construct comprises: a) a first ISVD that specifically binds to an IL-2Rβ subunit, an IL-2Rγ subunit, or both IL-2Rβγ subunits on the surface of the IL-2Rβγ positive cell; and b) a second ISVD that specifically binds to a target protein of interest, such that the bispecific ISVD construct binds to an IL-2Rβ subunit, an IL-2Rγ subunit, or both IL-2Rβγ subunits on the surface of the IL-2Rβγ positive cell and to the target protein.

In certain aspects, methods of the disclosure comprise contacting an IL-2Rβγ positive cell with a bispecific NANOBODY® ISVD (such as a V_{HH}, including a humanized V_{H} or a camelized V_{H}), wherein the bispecific NANOBODY® ISVD comprises: a) a first NANOBODY® ISVD (such as a V_{HH}, including a humanized V_{H} or a camelized V_{H}) that specifically binds to an IL-2Rβ subunit, an IL-2Rγ subunit, or both IL-2Rβγ subunits on the surface of the IL-2Rβγ positive cell; and b) a second NANOBODY® ISVD (such as a V_{HH}, including a humanized V_{H} or a camelized V_{H}) that specifically binds to the target protein of interest, such that the bispecific NANOBODY® ISVD binds to the an IL-2Rβ subunit, an IL-2Rγ subunit, or both IL-2Rβγ subunits on the surface of the IL-2Rβγ positive cell and to the target protein.

The SEQ ID NOs for the CDR sequences referred to in Table 4 are based on the CDR definition according to the AbM definition. It is noted that the CDR sequences defined according to the Kabat definition, Chotia definition or IMGT definition can likewise be used. Accordingly, the ISVDs provided by the present technology, specifically binding to IL2Rγ or IL2Rβ as described above using the AbM definition, can be also described using the Kabat definition, Chotia definition or IMGT definition.

In one embodiment, the ISVD comprise CDRs (Kabat definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360.

In one embodiment, the ISVD comprise CDRs (Chothia definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360.

In one embodiment, the ISVD comprise CDRs (IMGT definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360.

### ISVD Kabat Numbering

The total number of amino acid residues in each of the CDRs for V_{H} domains and for V_{HH} domains may vary and may not correspond to the total number of amino acid residues indicated by the Kabat numbering (e.g., one or more positions according to the Kabat numbering may not be occupied in the actual sequence, or the actual sequence may contain more amino acid residues than the number allowed for by the Kabat numbering). Consequently, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In Vivo Half-Life and ISVD that Increase In Vivo Half-Life

In certain aspects, a multispecific binding protein further comprises one or more groups, residues, moieties or binding units that provide the multispecific binding protein with enhanced therapeutic efficacy. For example, therapeutic efficacy can be enhanced by increasing the in vivo half-life (referred to also as “half-life”) of the multispecific binding protein described herein. In certain aspects, a multispecific binding protein further comprising one or more binding moieties operatively linked to the multispecific binding protein by a linker region, e.g., a linker encoded by any one of the amino acid sequences set forth in Table 3.

The term “in vivo half-life extension” means that the multispecific binding protein has an increased half-life in a mammal, such as a human subject, after administration. Half-life can be expressed, e.g., as t½beta. A multispecific binding protein half-life can be extended e.g., by a polyethylene glycol molecule, an Fc domain, a modified Fc domain, serum proteins or fragments thereof, serum binding proteins (e.g., an IgG), or binding moieties that can bind to serum proteins (e.g., an ISVD that binds a serum protein). A multispecific binding protein half-life can be extended e.g., by one or more binding units that facilitates binding to a FcRn.

In certain aspects, the binding unit that facilitates binding to a FcRn is selected from a group consisting of an Fc constant domain, a modified Fc domain, a serum albumin, a serum immunoglobulin, or a variant thereof. In certain aspects, the serum albumin is a human serum albumin or the serum immunoglobulin is an IgG.

In certain aspects, the binding unit is an ISVD that binds to human serum albumin e.g., an ISVD that binds to a human serum albumin described in WO 2004/041865, WO 2006/122787, WO 2006/122787, WO2012/175400, WO 2012/175741, WO 2015/173325, WO 2017/080850, WO 2017/085172, WO 2018/104444, WO 2018/134235, or WO 2018/134234, which are incorporated by reference herein in their entirety.

In certain aspects, an ISVD that binds to a human serum albumin is linked to the first or second binding moieties of the multispecific binding protein described herein. In certain aspects, the ISVD that binds to a human serum albumin extends the half-life of the multispecific binding protein relative to a reference multispecific binding protein that does not have said ISVD.

In certain aspects, an amino acid sequence encoding an ISVD that binds to human serum albumin exhibits a sequence identity of more than 85%, 90%, 95%, or 99% with an amino acid sequence selected from the group consisting of SEQ ID NOs: 77-91 or 424-433. In certain aspects, the amino acid sequence is selected from the group consisting of SEQ ID NOs: 77, 78, 81, 83 or 90. An amino acid sequence encoding the ISVD that binds to human serum albumin can exhibit a sequence identity of more than 85, 90%, 95%, or 99% with an amino acid sequence set forth in SEQ ID NO: 90.

In certain aspects, an ISVD binding to human serum albumin is linked to the C-terminal end of the multispecific binding protein so that the C-terminal end of the human serum albumin is an exposed C-terminal end. In certain aspects, the C-terminal end of the ISVD binding to human serum albumin comprises a C-terminal extender. In certain aspects, the C-terminal extender is a C-terminal alanine (A) or glycine (G) extension. In certain aspects, C-terminal alanine (A) or glycine (G) extension comprises 1-3 alanine or glycine residues, respectively.

In certain aspects, an ISVD capable of binding to human serum albumin is not linked to the C-terminal end of the multispecific binding protein and the C-terminal end of the human serum albumin is not an exposed C-terminal end. In certain aspects, the C-terminal sequence “VTVSS” (SEQ ID NO: 434) is directly followed by a linker region, e.g., a linker region of Table 3.

TABLE 2
Serum albumin binding ISVD sequences

| SEQ ID NO/ | |
|---|---|
| Description | Amino acid sequence |
| SEQ ID NO: 77 | EVQLVESGGGLVQPGNSLRLSCAASGFTFSSFGMSWVRQAPGKGLEWVSSISGSGS |
| Alb8 | DTLYADSVKGRFTISRDNAKTTLYLQMNSLRPEDTAVYYCTIGGSLSRSSQGTLVT |
| | VSS |
| SEQ ID NO: 78 | EVQLLESGGGLVQPGGSLRLSCAASGFTFRSFGMSWVRQAPGKGPEWVSSISGSGS |
| Alb23 | DTLYADSVKGRFTISRDNSKNTLYLQMNSLRPEDTAVYYCTIGGSLSRSSQGTLVT |
| | VSS |
| SEQ ID NO: 79 | EVQLVESGGGVVQPGNSLRLSCAASGFTFSSFGMSWVRQAPGKGLEWVSSISGSGS |
| Alb129 | DTLYADSVKGRFTISRDNAKTTLYLQMNSLRPEDTATYYCTIGGSLSRSSQGTLVT |
| | VSSA |
| SEQ ID NO: 80 | EVQLVESGGGVVQPGGSLRLSCAASGFTFRSFGMSWVRQAPGKGPEWVSSISGSGS |
| Alb132 | DTLYADSVKGRFTISRDNSKNTLYLQMNSLRPEDTATYYCTIGGSLSRSSQGTLVT |
| | VSSA |
| SEQ ID NO: 81 | EVQLVESGGGLVQPGNSLRLSCAASGFTFSSFGMSWVRQAPGKGLEWVSSISGSGS |
| Alb11 | DTLYADSVKGRFTISRDNAKTTLYLQMNSLRPEDTAVYYCTIGGSLSRSSQGTLVT |
| | VSS |
| SEQ ID NO: 82 | EVQLVESGGGLVQPGNSLRLSCAASGFTFSSFGMSWVRQAPGKGLEWVSSISGSGS |
| Alb11 (S112K) -A | DTLYADSVKGRFTISRDNAKTTLYLQMNSLRPEDTAVYYCTIGGSLSRSSQGTLVK |
| | VSSA |
| SEQ ID NO: 83 | EVQLVESGGGVVQPGNSLRLSCAASGFTFSSFGMSWVRQAPGKGLEWVSSISGSGS |
| Alb82 | DTLYADSVKGRFTISRDNAKTTLYLQMNSLRPEDTALYYCTIGGSLSRSSQGTLVT |
| | VSS |
| SEQ ID NO: 84 | EVQLVESGGGVVQPGNSLRLSCAASGFTFSSFGMSWVRQAPGKGLEWVSSISGSGS |
| Alb82-A | DTLYADSVKGRFTISRDNAKTTLYLQMNSLRPEDTALYYCTIGGSLSRSSQGTLVT |
| | VSSA |
| SEQ ID NO: 85 | EVQLVESGGGVVQPGNSLRLSCAASGFTFSSFGMSWVRQAPGKGLEWVSSISGSGS |
| Alb82-AA | DTLYADSVKGRFTISRDNAKTTLYLQMNSLRPEDTALYYCTIGGSLSRSSQGTLVT |
| | VSSAA |
| SEQ ID NO: 86 | EVQLVESGGGVVQPGNSLRLSCAASGFTFSSFGMSWVRQAPGKGLEWVSSISGSGS |
| Alb82-AAA | DTLYADSVKGRFTISRDNAKTTLYLQMNSLRPEDTALYYCTIGGSLSRSSQGTLVT |
| | VSSAAA |
| SEQ ID NO: 87 | EVQLVESGGGVVQPGNSLRLSCAASGFTFSSFGMSWVRQAPGKGLEWVSSISGSGS |
| Alb82-G | DTLYADSVKGRFTISRDNAKTTLYLQMNSLRPEDTALYYCTIGGSLSRSSQGTLVT |
| | VSSG |
| SEQ ID NO: 88 | EVQLVESGGGVVQPGNSLRLSCAASGFTFSSFGMSWVRQAPGKGLEWVSSISGSGS |
| Alb82-GG | DTLYADSVKGRFTISRDNAKTTLYLQMNSLRPEDTALYYCTIGGSLSRSSQGTLVT |
| | VSSGG |
| SEQ ID NO: 89 | EVQLVESGGGVVQPGNSLRLSCAASGFTFSSFGMSWVRQAPGKGLEWVSSISGSGS |
| Alb82-GGG | DTLYADSVKGRFTISRDNAKTTLYLQMNSLRPEDTALYYCTIGGSLSRSSQGTLVT |
| | VSSGGG |
| SEQ ID NO: 90 | EVQLVESGGGVVQPGGSLRLSCAASGFTFRSFGMSWVRQAPGKGPEWVSSISGSGS |
| Alb23002 | DTLYADSVKGRFTISRDNSKNTLYLQMNSLRPEDTALYYCTIGGSLSRSSQGTLVT |
| | VSS |
| SEQ ID NO: 91 | EVQLVESGGGVVQPGGSLRLSCAASGFTFRSFGMSWVRQAPGKGPEWVSSISGSGS |
| Alb223 | DTLYADSVKGRFTISRDNSKNTLYLQMNSLRPEDTALYYCTIGGSLSRSSQGTLVT |
| | VSSA |
| SEQ ID NO: 424 | QVQLVESGGGLVQPGGSLRLSCAASGFTFSDYWMYWVRQAPGKGLEWVSEINTNGL |
| Alb1 | ITKYPDSVKGRFTISRDNAKNTLYLQMNSLKPEDTALYYCARSPSGENRGQGTQVT |
| | VSS |
| SEQ ID NO: 425 | AVQLVESGGGLVQPGDSLRLSCAASGRTFSDYSGYTYTVGWFRQAPGKEREFVARI |
| Alb3 | YWSSGNTYYADSVKGRFTISRDIAKNTVDLLMNNLEPEDTAVYYCAARDGIPTSRS |
| | VESYNYWGQGTQVTVSS |
| SEQ ID NO: 426 | AVQLVDSGGGLVQAGGSLRLSCAASGRTFSAHSVYTMGWFRQAPGKEREFVARIYW |
| Alb4 | SSANTYYADSVKGRFTISRDNAKNTVDLLMNCLKPEDTAVYYCAARDGIPTSRSVE |
| | AYNYWGQGTQVTVSS |
| SEQ ID NO: 427 | QVQLVESGGGLVQAGGSLSLSCAASGRSFTGYYMGWFRQAPGKERQLLASISWRGD |
| Alb5 | NTYYKESVKGRFTISRDDAKNTIYLQMNSLKPEDTAVYYCAASILPLSDDPGWNTN |
| | WGQGTQVTVSS |
| SEQ ID NO: 428 | EVQLVESGGGLVQAGGSLSLSCSASGRSLSNYYMGWFRQAPGKERELLGNISWRGY |
| Alb6 | NIYYKDSVKGRFTISRDDAKNTIYLQMNRLKPEDTAVYYCAASILPLSDDPGWNTY |
| | WGQGTQVTVSS |
| SEQ ID NO: 429 | AVQLVESGGGLVQGGGSLRLACAASERIFDLNLMGWYRQGPGNERELVATCITVGD |
| Alb7 | STNYADSVKGRFTISMDYTKQTVYLHMNSLRPEDTGLYYCKIRRTWHSELWGQGTQ |
| | VTVSS |
| SEQ ID NO: 430 | EVQLVESGGGLVQEGGSLRLACAASERIWDINLLGWYRQGPGNERELVATITVGDS |
| Alb8 | TSYADSVKGRFTISRDYDKNTLYLQMNSLRPEDTGLYYCKIRRTWHSELWGQGTQV |
| | TVSS |
| SEQ ID NO: 431 | AVQLVESGGGLVQPGNSLRLSCAASGFTFRSFGMSWVRQAPGKEPEWVSSISGSGS |
| Alb9 | DTLYADSVKGRFTISRDNAKTTLYLQMNSLKPEDTAVYYCTIGGSLSRSSQGTQVT |
| | VSS |
| SEQ ID NO: 432 | AVQLVDSGGGLVQPGGSLRLSCAASGFSFGSFGMSWVRQYPGKEPEWVSSINGRGD |
| Alb10 | DTRYADSVKGRFSISRDNAKNTLYLQMNSLKPEDTAEYYCTIGRSVSRSRTQGTQV |
| | TVSS |
| SEQ ID NO: 433 | EVQLLESGGGLVQPGGSLRLSCAASGFTFRSFGMSWVRQAPGKGPEWVSSISGSGS |
| Alb 23 | DTLYADSVKGRFTISRDNSKNTLYLQMNSLRPEDTAVYYCTIGGSLSRSSQGTLVT |
| | VSS |
| SEQ ID NO: 434 | VTVSS |
| C-terminal end | |

### Relative Order of an ISVD in a Multispecific Binding Protein

The terms “first ISVD”, “second ISVD”, “third ISVD” as used herein primarily provide a reference point to more clearly define the binding properties of a given ISVD present in a multispecific binding protein. For example, a multispecific binding protein described herein can comprise: a first ISVD that specifically binds to IL-2Rβ, a second ISVD that specifically binds IL-2Rγ, and a third ISVD that blocks PD1/PD-L1 interaction. This nomenclature does not provide any limitation with respect to the relative N- to C-position of the ISVDs within a multispecific binding protein.

For example, the “first ISVD”, “second ISVD”, or “third ISVD” may be located at the N-terminal end, C-terminal end, or at any location between the N- and C-terminal ends of the amino acid sequence encoding the multispecific binding protein.

In certain aspects, the relative order of the first, the second, and the third ISVD within the multispecific binding protein from the N-(N) to C-(C) terminal domain are selected from the following orders:
(i): (N)-first ISVD-second ISVD-third ISVD-(C);
(ii): (N)-second ISVD-first ISVD-third ISVD-(C);
(iii): (N)-third ISVD-first ISVD-second ISVD-(C);
(iv): (N)-third ISVD-second ISVD-first ISVD-(C);
(v): (N)-first ISVD-third ISVD-second ISVD-(C); or
(vi): (N)-second ISVD-third ISVD-first ISVD-(C).

In certain aspects, the multispecific binding protein further comprises one or more binding moieties that enhance the therapeutic efficacy of the multispecific protein e.g., by increasing the in vivo half-life. In certain aspects, the one or more binding units comprise an ISVD that binds to a human serum albumin. In certain aspects, the ISVD binds to a human serum albumin.

In certain aspects, the ISVD can be placed in any order in the multispecific binding protein, such as before, after, or between the first ISVD, the second ISVD, or the third ISVD.

In certain aspects, an ISVD binding to human serum albumin is linked to the C-terminal end of the multispecific binding protein so that the C-terminal end of the human serum albumin is an exposed C-terminal end. In certain aspects, the C-terminal end of the ISVD binding to human serum albumin comprises a C-terminal extender. In certain aspects, the C-terminal extender is a C-terminal alanine (A) or glycine (G) extension. In certain aspects, C-terminal alanine (A) or glycine (G) extension comprises 1-3 alanine or glycine residues, respectively.

In certain aspects, an ISVD binding to human serum albumin is not linked to the C-terminal end of the multispecific binding protein and the C-terminal end of the human serum albumin is not an exposed C-terminal end. In certain aspects, the C-terminal sequence “VTVSS” (SEQ ID NO: 434) is directly followed by a linker region, e.g., a linker region of Table 3.

### Linker Region

In certain aspects, a multispecific binding protein of the disclosure comprises a linker, e.g., a polypeptide or a peptide linker. In certain aspects, a polypeptide linker can comprise or consist of a glycine or glycine-serine linker or another suitable linker. In an aspect, a linker can link a variable and constant domain in a multispecific binding protein of the disclosure. In another aspect, a linker can link a scFv polypeptide.

Examples of suitable linkers include a single glycine (Gly) residue; a diglycine peptide (Gly-Gly); a tripeptide (Gly-Gly-Gly); a peptide with four glycine residues (Gly-Gly-Gly-Gly) (SEQ ID NO: 1); a peptide with five glycine residues (Gly-Gly-Gly-Gly-Gly) (SEQ ID NO: 2); a peptide with six glycine residues (Gly-Gly-Gly-Gly-Gly-Gly) (SEQ ID NO: 3); a peptide with seven glycine residues (Gly-Gly-Gly-Gly-Gly-Gly-Gly) (SEQ ID NO: 4); a peptide with eight glycine residues (Gly-Gly-Gly-Gly-Gly-Gly-Gly-Gly) (SEQ ID NO: 5). Other combinations of amino acid residues can be used such as the peptide Gly-Gly-Gly-Gly-Ser (SEQ ID NO: 6) and the peptide Gly-Gly-Gly-Gly-Ser-Gly-Gly-Gly-Gly-Ser (SEQ ID NO: 7).

In certain aspects, a glycine serine linker comprises an amino acid sequence of the formula (Gly4Ser)n (SEQ ID NO: 56), wherein n is a positive integer (e.g., 1, 2, 3, 4, 5, 6, 7, 8, 9 or 10). In certain aspects, the glycine-serine linker is Gly4Ser (G4S) (SEQ ID NO: 6). In certain aspects, the glycine linker is (G4S)2 (SEQ ID NO: 7), (G4S)3 (SEQ ID NO: 57), or (G4S)4 (SEQ ID NO: 58). In certain aspects, the linker region comprises a poly-Glycine-Serine (G4S) (SEQ ID NO: 6) linker.

TABLE 3
Linker Amino Acid Sequence

| SEQ ID NO / | |
|---|---|
| Description | Amino acid sequence |
| SEQ ID NO: 1 | GGGG |
| SEQ ID NO: 2 | GGGGG |
| SEQ ID NO: 3 | GGGGGG |
| SEQ ID NO: 4 | GGGGGGG |
| SEQ ID NO: 5 | GGGGGGGG |
| SEQ ID NO: 6 | GGGGS |
| 5GS linker | |
| SEQ ID NO: 7 | GGGGS GGGGS |
| 10GS linker | |
| SEQ ID NO: 8 | GGGGGS |
| SEQ ID NO: 56 | (GGGGS) _{N} |
| SEQ ID NO: 57 | GGGGS GGGGS GGGGS |
| 15GS linker | |
| SEQ ID NO: 58 | GGGGS GGGGS GGGGS GGGGS |
| 20GS linker | |
| SEQ ID NO: 64 | AAA |
| 3A linker | |
| SEQ ID NO: 65 | SGGS GGS |
| 7GS linker | |
| SEQ ID NO: 66 | GGGGS GGS |
| 8GS linker | |
| SEQ ID NO: 67 | GGGGS GGGS |
| 9GS linker | |
| SEQ ID NO: 68 | GGGGS GGGGS GGGGS GGS |
| 18GS linker | |
| SEQ ID NO: 69 | GGGGS GGGGS GGGGS GGGGS GGGGS |
| 25GS linker | |
| SEQ ID NO: 70 | GGGGS GGGGS GGGGS GGGGS GGGGS |
| 30GS linker | GGGGS |
| SEQ ID NO: 71 | GGGGS GGGGS GGGGS GGGGS GGGGS |
| 35GS linker | GGGGS GGGGS |
| SEQ ID NO: 72 | GGGGS GGGGS GGGGS GGGGS GGGGS |
| 40GS linker | GGGGS GGGGS GGGGS |
| SEQ ID NO: 73 | EPKSCDKTHTCPPCP |
| G1 hinge | |
| SEQ ID NO: 74 | GGGGSGGGSEPKSCDKTHTCPPCP |
| 9GS (SEQ ID NO: | |
| 67) -G1 hinge | |
| SEQ ID NO: 75 | EPKTPKPQPAAA |
| Llama upper | |
| long hinge | |
| region | |
| SEQ ID NO: 76 | ELKTPLGDTTHTCPRCPEPKSCDTPPPCPRCPE |
| G3 hinge | PKSCDTPPPCPRCPEPKSCDTPPPCPRCP |

In certain aspects, the linker comprises an amino acid sequence which is at least 75, 80, 85, 90, 93, 95, 99% or more identical to the amino acid sequence of SEQ ID NO: 8.

In certain aspects, the linker comprises at least a portion of a hinge region (e.g., derived from an IgG1, IgG2, IgG3, or IgG4 molecule) and a series of glycine-serine amino acid residues.

In certain aspects, a multispecific binding protein comprises a second binding moiety which is operatively linked to a first cell surface binding moiety.

In certain aspects, a multispecific binding protein comprises an IL-2Rβ subunit, an IL-2Rγ subunit, or both IL-2Rβγ subunits specific variable domain(s) and is operatively linked to a first Fc domain polypeptide.

In certain aspects, a multispecific binding protein further comprises one or more binding moieties operatively linked to the first cell surface binding moiety or the second binding moiety that specifically binds to the target protein. In certain aspects, the operative linker is a peptide linker. In certain aspects, the peptide linker is at 90% identical to a peptide linker encoded by an amino acid sequence set forth in SEQ ID NOs: 1-8, 56-58 or 64-76.

In certain aspects, a multispecific binding protein further comprising one or more binding moieties (or units) that provide the multispecific binding protein with increased half-life, compared to a reference multispecific binding protein without said binding moieties. In certain aspects, the one or more binding units facilitates binding to a FcRn. In certain aspects, the binding unit that facilitates binding to a FcRn is selected from a group consisting of: a Fc constant domain, a modified Fc domain, a serum albumin, a serum immunoglobulin, or a variant thereof. In certain aspects, the serum albumin is a human serum albumin or the serum immunoglobulin is an IgG.

In certain aspects, a multispecific binding protein comprising a further binding unit is an ISVD that binds to the human serum albumin. In certain aspects, the ISVD that binds to human serum albumin is located proximal to the N- or C-terminal end of the multispecific binding protein. In certain aspects, an amino acid sequence encoding the ISVD that binds to human serum albumin exhibits a sequence identity of more than 90% with an amino acid sequence selected from the group consisting of SEQ ID NOs: 77-91 or 424-433. In certain aspects, the amino acid sequence is selected from the group consisting of SEQ ID NOs: 77, 78, 81, 83, 90. In certain aspects, a first ISVD, a second ISVD, a third ISVD, or further ISVD are linked to one another or another element in a multispecific binding protein described herein using a 20GS linker (SEQ ID NO: 58).

### Fc Domain

In some aspects, an IL-2Rβ subunit, an IL-2Rγ subunit, or both IL-2Rβγ subunits targeting multispecific binding protein can comprise a Fc domain. As used herein, the term “Fc domain” or “Fc region” (used interchangeably) is defined as the portion of a heavy chain constant region beginning in the hinge region just upstream of the papain cleavage site (i.e., residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The terms “Fc variant,” “modified Fc,” “engineered Fc” are interchangeably used herein refer to a molecule or sequence that is modified from a native Fc but still comprises a binding site for the salvage receptor, FcRn (neonatal Fc receptor). Exemplary Fc variants, and their interaction with the salvage receptor, are known in the art. A modified Fc domain can comprise a molecule or sequence that is humanized from a non-human native Fc. Furthermore, a native Fc comprises regions that can be removed because they provide structural features or biological activity that are not required for the multispecific binding protein compositions. Thus, the term “modified Fc domain” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

“Effector function” as used herein is meant a biochemical event that results from the interaction of an antibody Fc region with an Fc receptor or ligand. A “functional Fc region” possesses an “effector function” of a native sequence Fc region. Exemplary “effector functions” include antibody-dependent cell-mediated cytotoxicity (ADCC) or antibody-dependent cell-mediated phagocytosis (ADCP).

The term “EU index” refers to the EU numbering convention for the constant regions of an antibody, as described in Edelman, G M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. Unless otherwise stated, all antibody Fc region numbering employed herein corresponds to the EU numbering scheme, as described in Edelman et al. (Proc. Natl. Acad. Sci. 63(1): 78-85. 1969).

The Fc domain of an IL-2Rβ subunit, an IL-2Rγ subunit, or both IL-2Rβγ subunits targeting multispecific binding protein of the disclosure may be engineered to promote heterodimerization over homodimerization. For example, the heavy chain constant region of the first heavy-light chain pair may comprise a different amino acid sequence from the heavy chain constant region of the second heavy-light chain pair, wherein the different amino acid sequences are engineered to promote heterodimerization of the heavy chain constant regions. Examples include knobs-into-holes mutations or charge pair mutations. Alternatively, the heavy chain constant region of the first heavy-light chain pair may be identical to the heavy chain constant region of the second heavy-light chain pair, in which case it is expected that both homodimers and heterodimers will assemble, and these will be subsequently separated using one or more purification steps in the antibody manufacturing process to isolate the desired heterodimer comprising one anti-IL-2Rβγ arm and one anti-target protein arm.

In certain aspects, a first and/or second Fc constant domain is derived from an immunoglobulin class selected from a group consisting of: IgM, IgG, IgD, IgA, IgE. Chimeric Fc domains comprising portions of Fc domains from different species or Ig classes can also be employed. In certain aspects, a Fc constant domain is an IgG Fc domain, an IgG1 Fc domain, or an IgG4 Fc domain.

In certain aspects, methods for degrading a target protein are provided. The methods comprise contacting an IL-2Rβγ positive cell with a multispecific binding protein, wherein the multispecific binding protein comprises: a) a first cell surface binding moiety comprising an antibody or an antigen binding fragment linked to a first Fc domain polypeptide or variant thereof that binds to an IL-2Rβ subunit, an IL-2Rγ subunit, or both IL-2Rβγ subunits on the surface of the IL-2Rβγ positive cell; and b) a second binding moiety that specifically binds a target protein and comprises a second Fc domain polypeptide. In certain aspects the second binding moiety comprises an antibody or an antigen binding fragment linked to the second Fc domain polypeptide or a variant thereof. In certain aspects the first and/or second Fc domain can be modified.

In certain aspects, the first Fc domain can be engineered to pair or heterodimerize with the second Fc domain. For example, a multispecific binding protein of the disclosure may comprise a first polypeptide comprising a fusion of an IL-2Rβ subunit, an IL-2Rγ subunit, or both IL-2Rβγ subunits or an antigen binding fragment thereof and an Fc domain and a second polypeptide comprising a binding specificity (e.g., a VHH, Fab, or scFv) for a target protein of interest, wherein the second polypeptide comprises a second Fc domain capable of dimerizing with the first Fc domain. In certain aspects the first and/or second Fc domain can be modified.

### STY Mutation

Exemplary aspects of a modified glycan linked to a Fc domain via an engineered glycosylation site present on the Fc domain are described in WO2014043361A1 which is incorporated by reference in its entirety.

In certain aspects, a multispecific binding protein of the disclosure comprises a Fc domain that has an engineered N-linked glycosylation site. In certain aspects, a multispecific binding protein of the disclosure comprises one or more mutations or glycan modifications to modulate Fc mediated effector function. In certain aspects, a multispecific binding protein can comprise one or more mutations to modulate serum half-life.

In the case of a human IgG1 Fc domain, mutation of the wild-type amino acid at Kabat position 298 to an asparagine and Kabat position 300 to a serine or threonine results in the formation of an engineered N-linked glycosylation site (i.e, the N-X-T/S sequon, where X is any amino acid except proline). However, in the case of Fc domains of other species and/or Ig classes or isotypes, the skilled artisan will appreciate that it can be necessary to mutate Kabat position 299 of the Fc domain if a proline residue is present to recreate an N-X-T/S sequon.

In certain aspects, a multispecific binding protein comprises a constant domain comprising a Fc domain having an asparagine residue at amino acid position 298, according to EU numbering; and a serine or threonine residue at amino acid position 300, according to EU numbering. In certain aspects, a Fc domain further comprises an alanine residue at amino acid position 299, according to the EU numbering. In certain aspects, a Fc domain further comprises a glutamine residue at amino acid position 297, according to EU numbering.

In certain aspects, the at least one modified glycan is linked to an asparagine residue at amino acid position 298, according to EU numbering. In certain aspects, the at least one modified glycan is linked through a side chain of the asparagine residue through a β-glycosylamide linkage.

In certain aspects, a multispecific binding protein of the disclosure comprises mutations or glycan modifications to modulate Fc mediated effector function. In certain aspects, a multispecific binding protein comprises one or more mutations or glycan modifications to modulate serum half-life.

### Additional Fc Mutations

In certain aspects, a multispecific binding protein of the disclosure comprises a modified Fc domain and can further comprise one or more, e.g., two or more, three or more, or four or more, amino acid substitutions that confers a multispecific binding protein with one or more biochemical characteristics other than glycan modification.

Exemplary modified Fc domain amino acid substitutions that can confer additional biochemical characteristics to the multispecific binding proteins described herein are disclosed in WO2021016571A2, which is incorporated by reference in its entirety.

In certain aspects, a modified Fc region of a multispecific binding protein of the current disclosure comprises one or more mutations to modulate half-life (See e.g., Dall'Acqua et al. (2006) J Biol Chem 281: 23514-24, Zalevsky et al. (2010) Nat Biotechnol 28: 157-9, Hinton et al. (2004) J Biol Chem 279: 6213-6, Hinton et al. (2006) J Immunol 176: 346-56, Shields et al. (2001) J Biol Chem 276: 6591-604, Petkova et al. (2006) Int Immunol 18: 1759-69, Datta-Mannan et al. (2007) Drug Metab Dispos 35: 86-94, Vaccaro et al. (2005) Nat Biotechnol 23: 1283-8, Yeung et al. (2010) Cancer Res 70: 3269-77 and Kim et al. (1999) Eur J Immunol 29: 2819-25. (e.g., T250Q, M252Y, I253A, S254T, T256E, P2571, T307A, D376V, E380A, M428L, H433K, N434S, N434A, N434H, N434F, H435A and/or H435R).

In certain aspects, a modified Fc region of a multispecific binding protein of the current disclosure can have enhanced FcRn binding affinities at both an acidic pH (e.g., less than about 7.0, no more than about 6.5, or no more than about 6.0) and a non-acidic pH (e.g., no less than about 7.0, or no less than about 7.4), as compared to its wild-type. In another example, a multispecific binding protein comprising a modified Fc can comprise one or more amino acid mutations (e.g., substitutions) which alter the effector functions (e.g., ADCC or CDC function) of the Fc domain, as compared to a corresponding wild-type molecule, e.g., a molecule having the same structure as the FcRn antagonist except that it has a wild-type Fc domain. In another example, a multispecific binding protein can comprise a modified Fc domain comprising one or more amino acid mutations (e.g., substitutions) which alter (e.g., increase or decrease) the circulating half-life (e.g., serum half-life) of the FcRn antagonist, as compared to the corresponding wild-type molecule.

In certain aspects, a multispecific binding protein described herein can comprise a modified Fc domain that alters serum half-life compared to a multispecific binding protein comprising a wild-type Fc domain. In certain aspects, a modified Fc domain has an increased serum half-life compared to a multispecific binding protein comprising a wild-type Fc domain. In certain aspects, a Fc domain is modified to alter FcRn binding affinity compared to a multispecific binding protein comprising a wild-type Fc domain. In certain aspects, a modified Fc domain has enhanced FcRn binding affinity compared to a multispecific binding protein comprising a wild-type Fc domain. In certain aspects, a Fc domain is modified to enhance the FcRn binding affinity at an acidic pH compared to a multispecific binding protein comprising a wild-type Fc domain.

In certain aspects, a multispecific binding protein can have a modified Fc domain. In certain aspects, a multispecific binding protein can have a tyrosine (Y) at amino acid position 252, according to EU numbering. In certain aspects, a multispecific binding protein can have an aspartic acid (D) or a glutamic acid (E) at amino acid position 256, according to EU numbering. In certain aspects, a multispecific binding protein can have a tryptophan (W) or a glutamine (Q) at amino acid position 307, according to EU numbering. In certain aspects, a multispecific binding protein can have a phenylalanine (F) or a tyrosine (Y) at amino acid position 434; according to EU numbering.

In certain aspects, a multispecific binding protein can have a modified Fc domain comprising any combination of the following four amino acid residues: a tyrosine (Y) at amino acid position 252, an aspartic acid (D) or a glutamic acid (E) at amino acid position 256, a tryptophan (W) or a glutamine (Q) at amino acid position 307, and a phenylalanine (F) or a tyrosine (Y) at amino acid position 434; according to EU numbering.

In certain aspects, a multispecific binding protein can comprise a modified Fc domain having a combination of amino acid residues selected from the group consisting of: a) a tyrosine (Y) at amino acid position 252, an aspartic acid (D) at amino acid position 256, a glutamine (Q) at amino acid position 307, and a tyrosine (Y) at amino acid position 434; b) a tyrosine (Y) at amino acid position 252, a glutamic acid (E) at amino acid position 256, a tryptophan (W) at amino acid position 307, and a tyrosine (Y) at amino acid position 434; c) a tyrosine (Y) at amino acid position 252, a glutamic acid (E) at amino acid position 256, a glutamine (Q) at amino acid position 307, and a tyrosine (Y) at amino acid position 434; d) a tyrosine (Y) at amino acid position 252, an aspartic acid (D) at amino acid position 256, a glutamine (Q) at amino acid position 307, and a phenylalanine (F) at amino acid position 434; e) a tyrosine (Y) at amino acid position 252, an aspartic acid (D) at amino acid position 256, a tryptophan (W) at amino acid position 307, and a tyrosine (Y) at amino acid position 434; and f) a tyrosine (Y) at amino acid position 252, an aspartic acid (D) at amino acid position 256, a tryptophan (W) at amino acid position 307, and a phenylalanine (F) at amino acid position 434; according to EU numbering.

In certain aspects, a multispecific binding protein can comprise a modified Fc domain comprising a quadruple amino acid substitution selected from the group consisting of: M252Y/T256D/T307Q/N434Y, M252Y/T256E/T307W/N434Y, M252Y/T256E/T307Q/N434Y, M252Y/T256D/T307Q/N434F, M252Y/T256D/T307W/N434Y, and M252Y/T256D/T307W/N434F; according to EU numbering.

### Knobs and Holes

Techniques for making multispecific binding proteins (e.g., multispecific antibodies) include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al., EMBO J. 10 (1991) 3655-3659), and “knob-in-hole” engineering (see, e.g., U.S. Pat. No. 5,731,168). Multispecific binding proteins may also be made by engineering electrostatic steering effects for making binding protein Fc-heterodimeric molecules (WO 2009/089004); cross-linking two or more antibodies or fragments (see, e.g., U.S. Pat. No. 4,676,980, and Brennan, M. et al., Science 229 (1985) 81-83); using leucine zippers to produce bi-specific antibodies (see, e.g., Kostelny, S. A. et al., J. Immunol. 148 (1992) 1547-1553; using “diabody” technology for making multispecific binding protein fragments (see, e.g., Holliger, P. et al., Proc. Natl. Acad. Sci. USA 90 (1993) 6444-6448); and using single-chain Fv (scFv) dimers (see, e.g., Gruber, M et al., J. Immunol. 152 (1994) 5368-5374); and preparing trispecific binding proteins as described, e.g., in Tutt, A. et al., J. Immunol. 147 (1991) 60-69).

A wide variety of recombinant multispecific binding protein formats have been developed, e.g., by fusion of, e.g., an IgG binding protein format and single chain domains (see Kontermann R E, mAbs 4:2, (2012) 1-16). Multispecific binding proteins wherein the variable domains V_{L} and V_{H} or the constant domains CL and CH1 are replaced by each other are described in WO2009080251 and WO2009080252.

An approach to circumvent the problem of mispaired byproducts, is known as “knobs-into-holes”, aims at forcing the pairing of two different binding protein heavy chains by introducing mutations into the CH3 domains to modify the contact interface. On one chain bulky amino acids can be replaced by amino acids with short side chains to create a “hole.” Conversely, amino acids with large side chains can be introduced into the other CH3 domain, to create a “knob.” By co-expressing these two heavy chains (and two identical light chains, which have to be appropriate for both heavy chains), high yields of heterodimer formation (“knob-hole”) versus homodimer formation (“hole-hole” or “knob-knob”) was observed (Ridgway J B, Presta L G, Carter P; and WO1996027011). The percentage of heterodimer could be further increased by remodeling the interaction surfaces of the two CH3 domains using a phage display approach and the introduction of a disulfide bridge to stabilize the heterodimers (Merchant A. M, et al, Nature Biotech 16 (1998) 677-681; ATwell S, Ridgway J B, Wells J A, Carter P., J Mol Biol 270 (1997) 26-35). New approaches for the knobs-into-holes technology are described in e.g., in EP 1870459A1. Xie, Z., et al, J Immunol Methods 286 (2005) 95-101 refers to a format of multispecific binding protein using scFvs in combination with knobs-into-holes technology for the Fc part.

In certain aspects, the CH3 domains of the heavy chains of a multispecific binding protein can be altered by the “knob-into-holes” technology, which is described in detail with several examples in e.g., WO 96/027011, WO 98/050431, Ridgway J. B. et al., Protein Eng. 9 (1996) 617-621, Merchant A. M. et al., Nat Biotechnol 16 (1998) 677-681. In this method, the interaction surfaces of the two CH3 domains are altered to increase the heterodimerization of both heavy chains containing said two CH3 domains. Each of the two CH3 domains (of the two heavy chains) can be the “knob,” while the other is the “hole.” The introduction of a disulfide bridge can be utilized to stabilize the heterodimers (Merchant A. M et al., Nature Biotech 16 (1998) 677-681, Atwell, S. et al., J. Mol. Biol. 270 (1997) 26-35), as well as to increase the yield.

The Fc domain of a multispecific binding protein can be engineered to promote heterodimerization over homodimerization. For example, the heavy chain constant region of the first heavy-light chain pair can comprise a different amino acid sequence from the heavy chain constant region of the second heavy-light chain pair, wherein the different amino acid sequences are engineered to promote heterodimerization of the heavy chain constant regions. Examples include knobs-into-holes mutations or charge pair mutations. Alternatively, the heavy chain constant region of the first heavy-light chain pair may be identical to the heavy chain constant region of the second heavy-light chain pair, in which case it is expected that both homodimers and heterodimers will assemble, and these will be subsequently separated using one or more purification steps in the antibody manufacturing process to isolate the desired heterodimer comprising one anti-IL-2Rβγ arm and one anti-target protein arm.

In certain aspects, a multispecific binding protein of the disclosure comprises a first and a second IgG Fc domain polypeptide that dimerize to form the bispecific binding protein. In certain aspects, the first and second IgG Fc domain polypeptides dimerize by knobs-into-holes interactions, Fab arm exchange (FAE), electrostatic steering interactions, hydrophobic interactions, or any combination thereof. In certain aspects, the first IgG Fc domain polypeptide comprises a knob substitution, and the second IgG Fc domain polypeptide comprises a hole substitution. In certain aspects, a multispecific binding protein comprises a knob substitution that is selected from the group consisting of arginine (R), phenylalanine (F), tyrosine (Y), and tryptophan (W). In certain aspects, the multispecific binding protein comprises a hole substitution that is selected from the group consisting of alanine (A), asparagine (N), aspartic acid (D), glycine (G), serine (S), threonine (T), and valine (V).

### IL-2Rβγ Binding Moieties

The natural ligand for the IL-2 receptor (IL-2R) is IL-2. IL-2 (also known as the T cell growth factor) is a cytokine with pleiotropic effects on immune system which is also dictated by receptor affinity and engagement. IL-2R is found on T cells (both activated and regulatory T cells), activated B cells, some thymocytes, myeloid precursors and oligodendrocytes.

IL-2R is a multisubunit receptor that includes three major IL-2 binding subunits, the IL-2Rα (CD25) subunit, the IL-2Rβ (CD122) subunit, and the IL-2Rγ (CD132; also called γ_{c}) chains. The three receptor subunits can assemble in different combinations and orders to generate a low-, intermediate-, and high-affinity IL-2 receptor.

When IL-2Rα subunit associates with IL-2Rβ and IL-2Rγ subunits (i.e., IL-2Rβγ) it forms a heterotrimeric complex that acts as the high-affinity receptor for IL-2. The high-affinity IL-2R (IL-2Rαβγ) is expressed constitutively on e.g., CD4⁺FoxP3⁺ T-regulatory cells and transiently on activated T cells.

When only the IL-2Rβ and IL-2Rγ subunits associate in the absence of IL-2Rα it forms a heterodimeric complex that acts as the low or intermediate-affinity receptor for IL-2. The intermediate-affinity receptor (IL-2Rβγ) is expressed e.g., on resting T cells, CD8+ memory T cells, and NK cells.

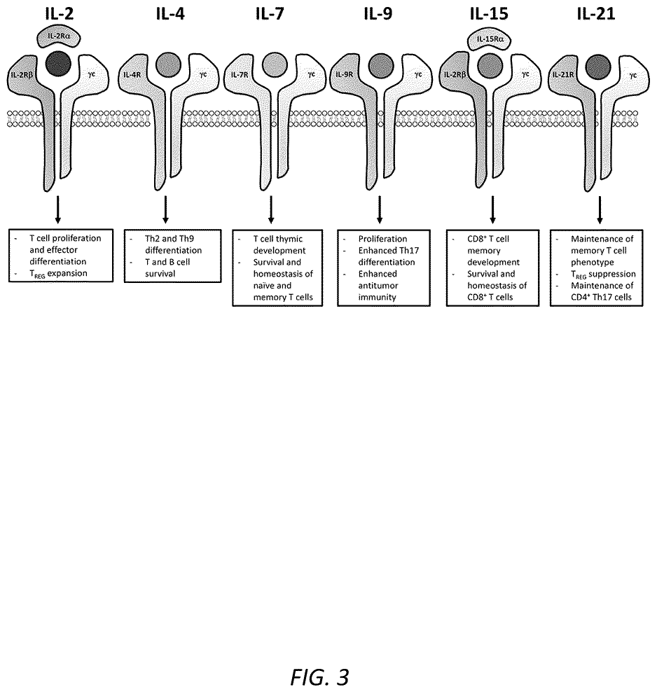

The IL-2Rβ and IL-2Rγ subunits can also combine with and in different combinations and orders to form functional receptors for other cytokines as shown in FIG. 3. For example, the IL-2Rβ and the IL2Rγ subunit can combine with IL-15Rα to form the heterotrimeric receptor for IL-15. In some aspects, a multispecific binding protein described herein (e.g., an immunoadhesin molecule) can bind to the IL2Rγ and/or the IL2Rβ subunit that is part of a IL-2R and/or IL-15R complex. In some aspects, a multispecific binding protein of the disclosure comprises a KD050 or KD050 variant thereof can bind to the IL2Rβ subunit or the IL2Rγ subunit that is part of a IL-2R or IL-15R complex.

In a different example, the IL-2Rγ subunit can combine with either IL-4Rα, IL-7Rα, IL-9Rα, or IL-21R to form the receptors for IL-4, IL-7, IL-9, and IL-21, respectively.

In some aspects, the IL-2Rβγ positive cell comprises either of a trimer consisting of the IL-2Rβ subunit, the IL-2Rγ subunit, and a CD25 (IL-2Rα) subunit (IL-2Rαβγ) or a dimer consisting of the IL-2Rβ subunit and the IL-2Rγ subunit (IL-2Rβγ).

In some aspects, a multispecific binding protein comprising a first cell surface binding moiety that binds to an IL-2Rβ subunit, an IL-2Rγ subunit, or both IL-2Rβγ subunits on the surface of the IL-2Rβγ positive cell, binds an extracellular domain of the IL-2Rβ subunit, the IL-2Rγ subunit, or both IL-2Rβγ subunits In some aspects, a first cell surface binding moiety comprises an IL-2Rβ subunit, an IL-2Rγ subunit, or both IL-2Rβγ subunits specific variable domain(s).

In certain aspects, methods for degrading a target protein are provided. The methods comprise contacting an IL-2Rβγ positive cell with a multispecific binding protein, wherein the multispecific binding protein comprises: a) a first cell surface binding moiety comprising an antibody or an antigen binding fragment linked to a first Fc domain polypeptide or variant thereof that binds to an IL-2Rβ subunit, an IL-2Rγ subunit, or both IL-2Rβγ subunits on the surface of the IL-2Rβγ positive cell; and b) a second binding moiety that specifically binds a target protein and comprises a second Fc domain polypeptide. In certain aspects the second binding moiety comprises an antibody or an antigen binding fragment linked to the second Fc domain polypeptide or a variant thereof. In certain aspects the first and/or second Fc domain can be modified.

In some aspects, an IL-2Rβ subunit, an IL-2Rγ subunit, or both IL-2Rβγ subunits specific variable domain(s) is operatively linked to a first Fc domain polypeptide. In some aspects, a first Fc domain polypeptide is an immunoglobulin G (IgG) Fc domain polypeptide.

In certain aspects, a multispecific binding protein of the disclosure comprises an IL-2Rβ subunit, an IL-2Rγ subunit, or both IL-2Rβγ subunits binding moiety, such as an antibody or an antigen binding fragment thereof that specifically binds an IL-2Rβ subunit, an IL-2Rγ subunit, or both IL-2Rβγ subunits on IL-2Rβγ positive cells to facilitate lysosomal targeting. In certain aspects, an anti-IL-2Rβ, an anti-IL-2Rγ, or an anti-IL-2Rβγ antibody or an antigen binding fragment thereof binds an extracellular domain of the IL-2Rβ subunit, the IL-2Rγ subunit, or both IL-2Rβγ subunits. In certain aspects, the IL-2Rβ subunit, the IL-2Rγ subunit, or both IL-2Rβγ subunits antibody or an antigen binding fragment thereof comprises a variable domain(s) that specifically binds to the IL-2Rβ subunit, the IL-2Rγ subunit, the IL-2Rβγ dimer, or the IL-2Rαβγ trimer. In certain aspects, the IL-2Rβ subunit, the IL-2Rγ subunit, or both IL-2Rβγ subunits antibody or an antigen binding fragment thereof comprising a variable domain is operatively linked to a first Fc domain polypeptide. In certain aspects, a first Fc domain polypeptide is an immunoglobulin G (IgG) Fc domain polypeptide.

In certain aspects, the multispecific binding protein of the disclosure comprises an amino acid sequence set forth in SEQ ID NO: 60. The amino acid sequence set forth in SEQ ID NO: 60 is:

DVQLVESGGGVVQPGGSLRLSCAATGFTLDYYVIGWFRQAPGKEREGVAC
LSSNGDRINYADSVKGRFTISRDIAKNTVYLQMNSLRPEDTALYHCAAGT
STTVRDMCGIMYLYDYWAQGTLVTVSSGGGGSGGGSEVQLVESGGGVVQP
GGSLRLSCAATGFTLDYYVIGWFRQAPGKEREGVACLSSNGDRINYADSV
KGRFTISRDIAKNTVYLQMNSLRPEDTALYHCAAGTSTTVRDMCGIMYLY
DYWAQGTLVTVSSGGGGSGGGGSGGGGSGGGGSEVQLVESGGGLVQAGDS
TTISCVASGGSFATYAFGWFRQAPGKEREFVATISQRGSTSYYSDSVEGR
FTISKDNAKSTVYLQMNSLQPEDTAVYYCAARYYGVDYRSTSYDFWGQGT
LVTVSSGGGGSGGGGSGGGGSGGGGSEVQLVESGGGLVQAGGSLRLSCVA
SGRTFSATNMGWFRQAPGKEREFVGIISESGRNTDYADSVKGRFTITRDN
AKSTVYLQMNNLKPEDTAVYYCALGRDYFATDTRQYNYWGLGTLVTVSSG
GGGSGGGGSGGGGSGGGGSEVQLVESGGGVVQPGGSLRLSCAASGFTFRS
FGMSWVRQAPGKGPEWVSSISGSGSDTLYADSVKGRFTISRDNSKNTLYL
QMNSLRPEDTALYYCTIGGSLSRSSQGTLVTVSSA.

In certain aspects, the multispecific binding protein of the disclosure comprises an amino acid sequence set forth in SEQ ID NO: 61. The amino acid sequence set forth in SEQ ID NO: 61 is:

DVQLVESGGGVVQPGGSLRLSCAATGFTLDYYVIGWFRQAPGKEREGVAC
LSSNGDRINYADSVKGRFTISRDIAKNTVYLQMNSLRPEDTALYHCAAGT
STTVRDMCGIMYLYDYWAQGTLVTVSSGGGGSGGGSEVQLVESGGGVVQP
GGSLRLSCAATGFTLDYYVIGWFRQAPGKEREGVACLSSNGDRINYADSV
KGRFTISRDIAKNTVYLQMNSLRPEDTALYHCAAGTSTTVRDMCGIMYLY
DYWAQGTLVTVSSGGGGGGGGSGGGGSGGGGSEVQLVESGGGWVQPGGSL
RLSCSPSERLSSIDIMGWYRQTPGKEREWVATITGDDTTHYENSVKGRFT
ISRDTAKNMVYLQMNSLEPEDTAVYYCNAGILPREEYWGQGTLVTVSSGG
GGSGGGGSGGGGSGGGGSEVQLVESGGGLVQAGGSLRLSCAASGRSFSSY
YMGWFRQAPGKEREFVAGINWSGRSTHYADSVKGRFTISRDNAKNTVYLQ
MNSLKPEDRAAYYCAADSAPSTRIGVRSLWDDYDYWGQGTLVTVSSGGGG
SGGGGSGGGGSGGGGSEVQLVESGGGVVQPGGSLRLSCAASGFTFRSFGM
SWVRQAPGKGPEWVSSISGSGSDTLYADSVKGRFTISRDNSKNTLYLQMN
SLRPEDTALYYCTIGGSLSRSSQGTLVTVSSA.

In certain aspects, the multispecific binding protein of the disclosure comprises an amino acid sequence set forth in SEQ ID NO: 62. The amino acid sequence set forth in SEQ ID NO: 62 is:

DVQLVESGGGVVQPGGSLRLSCAATGFTLDYYVIGWFRQAPGKEREGVAC
LSSNGDRINYADSVKGRFTISRDIAKNTVYLQMNSLRPEDTALYHCAAGT
STTVRDMCGIMYLYDYWAQGTLVTVSSGGGGSGGGSEVQLVESGGGVVQP
GGSLRLSCAATGFTLDYYVIGWFRQAPGKEREGVACLSSNGDRINYADSV
KGRFTISRDIAKNTVYLQMNSLRPEDTALYHCAAGTSTTVRDMCGIMYLY
DYWAQGTLVTVSSGGGGSGGGSEVQLVESGGGLVQAGTSLRLSCAASGTF
FRINDMGWYRQAPGRQRELVAYITSGGSTHYADSLKGRFTISRDNAKNTI
LLQMNSLKPEDTGVYYCYTDIEARLNEWRTVWGQGTLVTVSSGGGGSGGG
GSGGGGSGGGGSEVQLVESGGGSVQPGGSLRLSCTASGLTLDYYAIGWFR
QAPGKEREGVSCISSSSSDGSTLYADSVKGRFTISRDNDKNTVYLQLNSL
KPEDTGVYYCAADILNGECYSDYFEPLSGYDFDYWGQGTLVTVSSGGGGS
GGGGSGGGGSGGGGSEVQLVESGGGVVQPGGSLRLSCAASGFTFRSFGMS
WVRQAPGKGPEWVSSISGSGSDTLYADSVKGRFTISRDNSKNTLYLQMNS
LRPEDTALYYCTIGGSLSRSSQGTLVTVSSA.

In certain aspects, the multispecific binding protein of the disclosure comprises an amino acid sequence set forth in SEQ ID NO: 63. The amino acid sequence set forth in SEQ ID NO: 63 is:

DVQLVESGGGVVQPGGSLRLSCAATGFTLDYYVIGWFRQAPGKEREGVAC
LSSNGDRINYADSVKGRFTISRDIAKNTVYLQMNSLRPEDTALYHCAAGT
STTVRDMCGIMYLYDYWAQGTLVTVSSGGGGSGGGSEVQLVESGGGVVQP
GGSLRLSCAATGFTLDYYVIGWFRQAPGKEREGVACLSSNGDRINYADSV
KGRFTISRDIAKNTVYLQMNSLRPEDTALYHCAAGTSTTVRDMCGIMYLY
DYWAQGTLVTVSSGGGGSGGGSEVQLVESGGGLVQAGGALRLSCAVSRNI
FSNDVIAWYRQAPGKQREWIAQVSTDGSTQYAHSVKGRFTISRDSAGNTV
YLRMDSLKPEDTAVYLCNTYLPSIPWGQGTLVTVSSGGGGSGGGSEVQLV
ESGGGLVQAGGSLSLSCLGSGPTFSEYPIGWFRQAPGMEREFVAAISGSG
GRPYFADSVKGRFSVSRDNAKNTVYLQMNSLKPEDTAVYYCAALNYYSSA
GTHSTPAYWGQGTQVTVSSGGGGSGGGGSGGGGSGGGGSEVQLVESGGGV
VQPGGSLRLSCAASGFTFRSFGMSWVRQAPGKGPEWVSSISGSGSDTLYA
DSVKGRFTISRDNSKNTLYLQMNSLRPEDTALYYCTIGGSLSRSSQGTLV
TVSSA.

In certain aspects, the multispecific binding protein comprises at least three ISVDs, wherein the at least three ISVDs comprise: a first ISVD that specifically binds to IL-2Rβ, a second ISVD that specifically binds IL-2Rγ, and a third ISVD that binds to a target protein. In certain aspects, a combination of the first and the second ISVD has an agonistic effect on IL-2Rβγ.

In certain aspects, the two subunits of the IL-2Rβ and IL-2Rγ comprise amino acid sequences corresponding to amino acid sequences of SEQ ID NO: 390 and SEQ ID NO: 391, respectively.

The amino acid sequence set forth in SEQ ID NO: 390 is:

MAAPALSWRLPLLILLLPLATSWASAAVNGTSQFTCFYNSRANISCVWSQ
DGALQDTSCQVHAWPDRRRWNQTCELLPVSQASWACNLILGAPDSQKLTT
VDIVTLRVLCREGVRWRVMAIQDFKPFENLRLMAPISLQVVHVETHRCNI
SWEISQASHYFERHLEFEARTLSPGHTWEEAPLLTLKQKQEWICLETLTP
DTQYEFQVRVKPLQGEFTTWSPWSQPLAFRTKPAALGKDTIPWLGHLLVG
LSGAFGFIILVYLLINCRNTGPWLKKVLKCNTPDPSKFFSQLSSEHGGDV
QKWLSSPFPSSSFSPGGLAPEISPLEVLERDKVTQLLLQQDKVPEPASLS
SNHSLTSCFTNQGYFFFHLPDALEIEACQVYFTYDPYSEEDPDEGVAGAP
TGSSPQPLQPLSGEDDAYCTFPSRDDLLLFSPSLLGGPSPPSTAPGGSGA
GEERMPPSLQERVPRDWDPQPLGPPTPGVPDLVDFQPPPELVLREAGEEV
PDAGPREGVSFPWSRPPGQGEFRALNARLPLNTDAYLSLQELQGQDPTHL
V.

The amino acid sequence set forth in SEQ ID NO: 391 is:

MLKPSLPFTSLLFLQLPLLGVGLNTTILTPNGNEDTTADFFLTTMPTDSL
SVSTLPLPEVQCFVFNVEYMNCTWNSSSEPQPTNLTLHYWYKNSDNDKVQ
KCSHYLFSEEITSGCQLQKKEIHLYQTFVVQLQDPREPRRQATQMLKLQN
LVIPWAPENLTLHKLSESQLELNWNNRFLNHCLEHLVQYRTDWDHSWTEQ
SVDYRHKFSLPSVDGQKRYTFRVRSRFNPLCGSAQHWSEWSHPIHWGSNT
SKENPFLFALEAVVISVGSMGLIISLLCVYFWLERTMPRIPTLKNLEDLV
TEYHGNFSAWSGVSKGLAESLQPDYSERLCLVSEIPPKGGALGEGPGASP
CNQHSPYWAPPCYTLKPET.

In certain aspects, an IL-2Rβγ is an endogenous cell membrane-bound surface receptor expressed on an immune cell, a white blood cell, or a hematopoietic cell. In certain aspects, an IL-2Rβγ is a cell membrane-bound surface receptor expressed on a neoplastic cell. In certain aspects, an IL-2Rβγ is an endogenous cell membrane-bound surface receptor expressed on a T-cell or a NK cell or a B cell. In certain aspects, an IL-2Rβγ positive cell is an immune cell, a white blood cell, a hematopoietic cell, a neoplastic cell, a T-cell, a NK cell, or a B cell. In certain aspects an IL-2Rβγ positive cell is a CD4+ T cell, a CD8+ T cell, or a NK cell.

Exemplary IL-2Rβ subunit, IL-2Rγ subunit, or both IL-2Rβγ subunits binding moieties can be derived from the IL-2Rβ subunit, the IL-2Rγ subunit, or both IL-2Rβγ subunits antibodies that are obtained by immunizing mice with native IL-2Rβ subunit, IL-2Rγ subunit, or both IL-2Rβγ subunits or a full-length recombinant IL-2Rβ subunit, IL-2Rγ subunit, or both IL-2Rβγ subunits peptide(s).

Exemplary IL-2Rβ subunit, IL-2Rγ subunit, or both IL-2Rβγ subunits binding moieties can be derived from the IL-2Rβ subunit, the IL-2Rγ subunit, or both IL-2Rβγ subunits ISVDs (such as V_{HH}) that are obtained by immunizing llamas with native IL-2Rβ subunit, IL-2Rγ subunit, or both IL-2Rβγ subunits or a full-length recombinant IL-2Rβ subunit, IL-2Rγ subunit, or both IL-2Rβγ subunits peptide(s).

Exemplary IL-2Rβ subunit, IL-2Rγ subunit, or both IL-2Rβγ subunits binding moieties can be derived from the IL-2Rβ subunit, the IL-2Rγ subunit, or both IL-2Rβγ subunits antibodies that are obtained by immunizing mice with native IL-2Rβ subunit, IL-2Rγ subunit, or both IL-2Rβγ subunits or a full-length recombinant IL-2Rβ subunit, IL-2Rγ subunit, or both IL-2Rβγ subunits peptide(s).

Alternatively, the IL-2Rβ subunit, the IL-2Rγ subunit, or both IL-2Rβγ subunits or a fragment thereof can be produced using biochemical techniques and modified and used as immunogen. In certain aspects, an immunogen can be a peptide from the N-terminal or C-terminal end of the IL-2Rβ subunit, the IL-2Rγ subunit, or both IL-2Rβγ subunits. In certain aspects, an immunogen can be a recombinant IL-2Rβ subunit, IL-2Rγ subunit, or both IL-2Rβγ subunits peptide(s) expressed in a prokaryote, such as E. coli, or in eukaryotic cells or mammalian cells such as Chinese hamster ovary (CHO) cells. In certain aspects, an extracellular domain of the human IL-2Rβ subunit, the IL-2Rγ subunit, or both IL-2Rβγ subunits, the IL-2Rβγ dimer, or the IL-2Rαβγ trimer can be used to raise the anti-IL-2Rβ, the anti-IL-2Rγ subunit, or the anti-IL-2Rβγ antibody. In certain aspects, the anti-IL-2Rβ, the anti-IL-2Rγ, or the anti-IL-2Rβγ antibody can be obtained by immunizing a transgenic mouse that expresses the human immune repertoire (e.g., the VELOCIMMUNE® mouse from Regeneron). The VELOCIMMUNE® mouse comprises a genome comprising human heavy and light chain variable regions operably linked to endogenous mouse constant region loci such that the mouse produces antibodies comprising a human variable region and a mouse constant region in response to antigenic stimulation. The DNA encoding the variable regions of the heavy and light chains of the anti-IL-2Rβ, the anti-IL-2Rγ, or the anti-IL-2Rβγ antibody can be isolated. The amino acids making up the variable regions can be incorporated into the multispecific binding proteins of the disclosure.

In certain aspects, an IL-2Rβ subunit, an IL-2Rγ subunit, or both IL-2Rβγ subunits binding moiety comprises the variable domains of an anti-IL-2Rβ, an anti-IL-2Rγ, or an anti-IL-2Rβγ antibody known in the art. For example, the multispecific binding protein of the disclosure can comprise an IL-2Rβ subunit, an IL-2Rγ subunit, or both IL-2Rβγ subunits binding moiety comprising the amino acid sequences of a known, commercially available anti-IL-2Rβ, anti-IL-2Rγ subunit, or anti-IL-2Rβγ binding protein. In other aspects, an anti-IL-2Rβ, anti-IL-2Rγ subunit, or anti-IL-2Rβγ binding moiety is the bioequivalent of a known binding protein. A bioequivalent IL-2Rβ subunit, an IL-2Rγ subunit, or both IL-2Rβγ subunits binding protein can comprise amino acid sequences that vary from those of a known IL-2Rβ subunit, IL-2Rγ subunit, or both IL-2Rβγ subunits binding proteins, but that retain the ability to bind an IL-2Rβ subunit, an IL-2Rγ subunit, or both IL-2Rβγ subunits. Such variant IL-2Rβ subunit, IL-2Rγ subunit, or both IL-2Rβγ subunits binding proteins comprise one or more additions, deletions, or substitutions of amino acids when compared to a parent sequence but exhibit biological activity that is essentially equivalent to that of the known IL-2Rβ subunit, IL-2Rγ subunit, or both IL-2Rβγ subunits binding protein. Variant IL-2Rβ subunit, an IL-2Rγ subunit, or both IL-2Rβγ subunits binding proteins are considered bioequivalent if, for example, they are pharmaceutical equivalents or pharmaceutical alternatives whose rate and extent of absorption do not show a significant difference when administered at the same molar dose under similar experimental conditions, either as a single dose or multiple doses. Some IL-2Rβ subunit, IL-2Rγ subunit, or both IL-2Rβγ subunits binding proteins will be considered equivalents or pharmaceutical alternatives if they are equivalent in the extent of their absorption but not in their rate of absorption and yet can be considered bioequivalent because such differences in the rate of absorption are intentional and are reflected in the labeling, are not essential to the attainment of effective body drug concentrations on, e.g., chronic use, and are considered medically insignificant for the particular drug product studied. Bioequivalent variants of known IL-2Rβ subunit, IL-2Rγ subunit, or both IL-2Rβγ subunits antibodies can be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent IL-2Rβ subunit, IL-2Rγ subunit, or both IL-2Rβγ subunits binding moieties can include variants comprising amino acid changes, which modify the glycosylation characteristics of known IL-2Rβ subunit, IL-2Rγ subunit, or both IL-2Rβγ subunits binding proteins e.g., mutations that eliminate or remove glycosylation.

In general, multispecific binding proteins as described herein can function by binding to an IL-2Rβ subunit, an IL-2Rγ subunit, or both IL-2Rβγ subunits and a target protein with high affinity or avidity. In certain aspects, a multispecific binding protein can bind an IL-2Rβ subunit, an IL-2Rγ subunit, or both IL-2Rβγ subunits and/or a target protein with a K_{D} of less than about 1 μM as measured by surface plasmon resonance (e.g., at 25° C. or at 37° C.). In certain aspects, the multispecific binding proteins bind an IL-2Rβ subunit, an IL-2Rγ subunit, or both IL-2Rβγ subunits and/or the target protein with a K_{D} of less than about 40 nM, less than about 30 nM, less than about 20 nM, less than about 10 nM less than about 5 nM, less than about 2 nM or less than about 1 nM, as measured by surface plasmon resonance.

In certain aspects, multispecific binding proteins described herein bind IL-2Rβ subunit, an IL-2Rγ subunit, or both IL-2Rβγ subunits with a dissociative half-life (t½) of greater than about 1.1 minutes as measured by surface plasmon resonance at, e.g., about 25° C. or 37° C. In certain aspects, the multispecific binding proteins bind an IL-2Rβ subunit, an IL-2Rγ subunit, or both IL-2Rβγ subunits and the soluble target protein with a t½ of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1200 minutes, as measured by surface plasmon resonance at 25° C. or 37° C.

In certain aspects, the multispecific binding proteins described herein comprise a modified binding moiety to alter binding affinity compared to a multispecific binding protein comprising a wild-type binding moiety. In certain aspects, a modified binding moiety has an enhanced binding affinity compared to a multispecific binding protein comprising a wild-type binding moiety. In certain aspects, a binding moiety is modified to enhance the binding affinity at an acidic pH compared to a multispecific binding proteins comprising a wild-type binding moiety. In certain aspects, a binding moiety is modified to enhance the binding affinity at a basic pH compared to a multispecific binding proteins comprising a wild-type binding moiety. In certain aspects, a modified binding moiety has a decreased binding affinity compared to a multispecific binding protein comprising a wild-type binding moiety. In certain aspects, a binding moiety is modified to decrease the binding affinity at an acidic pH compared to a multispecific binding protein comprising a wild-type binding moiety. In certain aspects, a binding moiety is modified to decrease the binding affinity at a basic pH compared to a multispecific binding protein comprising a wild-type binding moiety. In certain aspects, the first cell surface binding moiety of the multispecific binding protein binds to the IL-2Rβ subunit, the IL-2Rγ subunit, or both the IL-2Rβγ subunits on the surface of a IL-2Rβγ positive cell with an affinity from about 100 pM to about 1 μM (e.g., about 100 pM to about 1,000 pM, about 1,000 pM to about 0.01 μM, about 0.01 μM to about 0.1 μM, or about 0.1 μM to about 1.0 μM). In certain aspects, the second binding moiety of the multispecific binding protein binds to a target protein with an affinity from about 100 pM to about 1 μM (e.g., about 100 pM to about 1,000 pM, about 1,000 pM to about 0.01 μM, about 0.01 μM to about 0.1 μM, or about 0.1 μM to about 1.0 μM).

In certain aspects, a multispecific binding protein of the disclosure binds to an IL-2Rβ subunit, an IL-2Rγ subunit, or both IL-2Rβγ subunits on an IL-2Rβγ positive cell with an affinity from about 100 pM to about 1 μM.

### pH Sensitive IL-2Rβγ Binding

In certain aspects, multispecific binding proteins of the disclosure comprise an IL-2Rβ subunit, an IL-2Rγ subunit, or both IL-2Rβγ subunits binding moiety which exhibits pH-sensitive binding to an IL-2Rβ subunit, an IL-2Rγ subunit, or both IL-2Rβγ subunits. In certain aspects, the pH-sensitive binding moiety facilitates dissociation from IL-2Rβγ within the lysosomal compartment, allowing IL-2Rβγ and/or the multispecific binding protein to recycle back to the cell surface where it can bind additional target protein for degradation.

In certain aspects, methods for degrading a target protein are provided.

The methods can comprise contacting an IL-2Rβγ positive cell with a multispecific binding protein, wherein the multispecific binding protein comprises: a) a first cell surface binding moiety that specifically binds to a CD122 (IL-2Rβ) subunit and/or a CD132 (IL-2Rγ) subunit, on the surface of an IL-2Rβγ positive cell, wherein the first cell surface binding moiety comprises an immunoglobulin domain; and b) a second binding moiety that is operatively linked to the first cell surface binding moiety and that specifically binds to a target protein, wherein specific binding of the multispecific binding protein to the IL-2Rβ subunit and/or the IL-2Rγ subunit facilitates internalization of the target protein bound to the multispecific binding protein into the IL-2Rβγ positive cell.

In certain aspects, the IL-2Rβγ positive cell comprises either of a trimer consisting of the IL-2Rβ subunit, the IL-2Rγ subunit, and an IL-2Rα subunit (IL-2Rαβγ) or a dimer consisting of the IL-2Rβ subunit and the IL-2Rγ subunit (IL-2Rβγ).

After dissociation in a pH-dependent manner the multispecific binding protein (e.g., an IL-2Rβ subunit, an IL-2Rγ subunit, or both IL-2Rβγ subunits antibody or an antigen binding fragment thereof) can be recycled back to the cell surface membrane whereby the multispecific binding protein (e.g., IL-2Rβ subunit, IL-2Rγ subunit, or both IL-2Rβγ subunits antibody or antigen binding fragment thereof or an IL-2Rβ subunit, an IL-2Rγ subunit, or both IL-2Rβγ subunits ISVD) is released back into the circulation. Concomitantly, during dissociation, the IL-2Rβ subunit, the IL-2Rγ subunit, and/or the IL-2Rβγ heterodimer is not recycled back to the surface of the cell independent of the target protein.

Like IL-2Rβγ, the target protein continues along the late endocytic pathway for ultimate lysosomal degradation. In this way, an IL-2Rβ subunit, an IL-2Rγ subunit, or both IL-2Rβγ subunits bound to the multispecific binding protein of the disclosure can act as a shuttling mechanism for taking target proteins to the lysosomal degradation pathway.

The IL-2Rβ subunit, IL-2Rγ subunit, or both IL-2Rβγ subunits binding moiety can comprise a Fab domain comprising one or more mutations which enhance or diminish binding to an IL-2Rβ subunit, an IL-2Rγ subunit, or both IL-2Rβγ subunits under different pH conditions e.g., at acidic pH as compared to neutral pH. For example, an IL-2Rβ subunit, an IL-2Rγ subunit, or both IL-2Rβγ subunits binding portion of the multispecific antibody can comprise a mutation in the CH1, CL, V_{H}, or the V_{L} region of the Fab domain. The mutation(s) can increase the affinity of the Fab domain to its antigen in an acidic environment (e.g., in tumor microenvironment where pH is about 7.2, 7.0, 6.8, 6.5, 6.3 or lower). Such mutations can result in an increase in serum half-life of the multispecific binding protein when administered to a subject.

In certain aspects, the sensitivity of IL-2Rβ subunit, IL-2Rγ subunit, or both IL-2Rβγ subunits binding at acidic pH may be increased, whereby the IL-2Rβ subunit, IL-2Rγ subunit, or both IL-2Rβγ subunits binding moiety demonstrates reduced binding to an IL-2Rβ subunit, an IL-2Rγ subunit, or both IL-2Rβγ subunits at lower pH. In certain aspects, binding to an IL-2Rβ subunit, an IL-2Rγ subunit, or both IL-2Rβγ subunits can be reduced at a pH that reflects the endosomal compartment. In certain aspects, binding to an IL-2Rβ subunit, an IL-2Rγ subunit, or both IL-2Rβγ subunits is reduced at pH 5.5 relative to binding at neutral pH (pH 7.0). Such reduced binding at pH 5.5 may be 50% or more of the IL-2Rβ subunit, the IL-2Rγ subunit, or both IL-2Rβγ subunits binding observed at neutral pH. A change, such as a reduction or increase, in an anti-IL-2Rβ, IL-2Rγ, or IL-2Rβγ activity described herein, such as binding, may be in comparison to a reference or wild-type antibody. The reference antibody may be an anti-IL-2Rβ, anti-IL-2Rγ, or anti-IL-2Rβγ antibody, ISVD, or conjugate known in the art. The change may also be relative between two different pH levels of a particular antibody composition described herein. pH sensitive anti-IL-2Rβ, anti-IL-2γ, or anti-IL-2Rβγ antibodies may be identified by testing the interaction between plate coated IL-2Rβ, IL-2Rγ, or IL-2Rβγ and soluble anti-IL-2Rβ, anti-IL-2Rγ, or anti-IL-2Rβγ antibodies over a pH range of 4.5 to 7.0, and selecting antibodies with increased pH sensitivity such that reduced binding is observed at acidic pH. Exemplary embodiments of an anti-IL-2Rβ, anti-IL-2Rγ, or anti-IL-2Rβγ antibody with reduced binding to an IL-2Rβ subunit, an IL-2Rγ subunit, or both IL-2Rβγ subunits at acidic pH comprises the replacement of tyrosine with histidine within or near one or more CDR1-3 regions of at least one of a light chain and heavy chain variable region of the antibody. See WO2020214748A1, incorporated by reference in its entirety. In certain aspects, a multispecific binding protein of the disclosure exhibits pH-dependent binding to an IL-2Rβ subunit, an IL-2Rγ subunit, or both IL-2Rβγ subunits on an IL-2Rβγ positive cell.

### Target Proteins

A multispecific binding protein of the disclosure comprise a binding moiety which binds a target protein of interest. As the skilled artisan will appreciate, when paired with the first IL-2Rβ subunit, IL-2Rγ subunit, or both IL-2Rβγ subunits binding moiety to form a multispecific binding protein of the disclosure, the second binding moiety of the multispecific binding protein can facilitate the internalization and lysosomal degradation of a target protein of interest to which it binds. By specifically binding to a target protein, the multispecific binding protein enables its internalization by the IL-2Rβγ positive cell. The internalized target protein is then transported to the lysosomal compartment, where it undergoes degradation. This mechanism provides a means to target and degrade various target proteins or target proteins.

As used herein a “target protein” can be a protein having a deleterious function and for which degradation can be therapeutically advantageous. In certain aspects, a target protein is a membrane-associated target protein, a soluble target protein, or both. In certain aspects, a target protein is a pathogenic protein or a peptide which causes a disease or symptom of disease. In certain aspects, a target protein is an immune checkpoint protein, a cancer antigen, and/or an immunomodulatory protein. In certain aspects, a target protein is associated with a disease selected from the group consisting of: a neoplastic disorder (e.g., cancer), an autoimmune disease, an inflammatory disorder, an infectious disease, and a neurodegenerative disorder.

In certain aspects, a target protein is a soluble protein. In certain aspects, a target protein is a membrane target protein. In certain aspects, a target protein is expressed on the surface of the same or a different IL-2Rβγ positive cell. In other aspects, the target protein is expressed on the surface of a non-IL-2Rβγ positive cell (e.g., an antigen presenting cell).

In certain aspects, a membrane-associated target protein is expressed on the surface of a neoplastic cell and/or an immune cell. In certain aspects, a membrane-associated target protein is expressed on a T-cell. In certain aspects, the T-cell is an activated T cell or a regulatory T (Treg) cell.

In other aspects, a target protein is a soluble protein. Exemplary target proteins or peptides include proteins or peptides secreted by tumors, inflammatory protein or peptides, signaling molecules including cytokines, interleukins, interferons, tumor necrosis factors, growth factors, hormones, neurotransmitters, lipid mediators, activating factors, extracellular matrix (ECM) proteins, Wnt proteins, members of the Transforming Growth Factor-beta (TGF-β) family, and Notch ligands. In certain aspects, the target protein is selected from the group consisting of: an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein.

In certain aspects, a target protein is an antigen. Antigens are molecules that can elicit an immune response. In certain aspects, an antigen is an autoantigen or self-antigen produced in the cell of a subject. For example, an antigen could be a surface marker expressed on specific cell types, allowing a multispecific binding protein of the disclosure to selectively target and modulate those cells. By engaging an IL-2Rβγ positive cell via an IL-2Rβ subunit, IL-2Rγ subunit, or both IL-2Rβγ subunits binding portion, antigen-targeting multispecific binding proteins of the disclosure can enhance immune responses, facilitate cell-mediated cytotoxicity, or regulate immune cell functions in immunotherapy.

In other aspects, a target protein is an antibody (e.g., an autoantibody) or fragment thereof. An autoantibody is an antibody that specifically binds to one or more antigens made or formed by a subject's own body. Autoantibodies mistakenly recognize and target self-antigens, leading to autoimmune diseases. By binding autoantibodies as the second binding moiety, a multispecific binding protein, or a binding fragment thereof can specifically bind to the self-antigens associated with autoimmune disorders. This approach offers the potential for targeted therapy by redirecting the immune response towards the autoreactive cells or molecules involved in the autoimmune process.

By targeting specific proteins, a multispecific binding protein of the disclosure can, e.g., interfere with protein-protein interactions, disrupt signaling pathways, or block protein-mediated cellular functions. Membrane proteins are a class of proteins located within or associated with cellular membranes, playing, e.g., roles in cell signaling, transport of molecules across membranes, and maintaining the structural integrity of the cell. Membrane proteins can be attached to a cell membrane.

Membrane proteins can be buried in a cell membrane or can be anchored on to a cell membrane. In certain aspects, a target protein is membrane protein.

In certain aspects, the target protein is a soluble protein. Soluble proteins are a class of proteins that readily dissolve in aqueous environments or in extracellular fluid, maintaining stability and performing diverse functions within both cellular and tissue contexts. Soluble proteins can be proteins that are not membrane bound. In certain aspects, a target protein is a soluble protein.

In certain aspects, a target protein is associated with a disease or disorder where aberrant protein signaling is involved, such as certain cancers or metabolic disorders. A multispecific binding protein of the disclosure may be designed to target proteins with high affinity and selectivity enabling precise modulation of the aberrant signaling pathway.

In certain aspects, a target protein is a pathogenic protein. Pathogenic proteins are those that are associated with disease development or progression. By facilitating degradation of target pathogenic protein a multispecific binding protein of the disclosure can neutralize their activity, inhibit their binding to receptors or other molecules, or facilitate their clearance from the body. This approach is relevant in the field of, e.g., infectious diseases, or chronic infectious diseases such as such as Herpes viral infection (HSV, CMV, EBV), HIV-1, and HBV infections. In some aspects, multispecific binding proteins may be used to treat chronic viral infection. In certain aspects, multispecific binding proteins of the current disclosure can be designed to target viral or bacterial proteins involved in pathogenesis. By blocking or neutralizing pathogenic proteins, a multispecific binding protein, can help control the spread of the disease and limit its impact on the host. In certain aspects, a multispecific binding protein can comprise the variable domains of, be co-administered with or fused to an agent targeting an infectious disease target of interest. In certain aspects, the agent can be Palivizumab (e.g., to target the fusion (F) glycoprotein).

In certain aspects, a target protein can be a protein secreted by tumors. Tumor cells can release proteins that contribute to tumor growth such as growth factors, angiogenesis, immune evasion, or metastasis. A multispecific binding protein that target tumor secreted protein can interfere with their function, inhibit tumor-promoting activities, or enhance anti-tumor immune responses. This approach offers the potential for targeted therapy against cancer by specifically neutralizing or modulating tumor-secreted proteins that play critical roles in tumorigenesis and progression. In certain aspects, a target protein secreted by tumors of the present disclosure is Vascular endothelial growth factor A (VEGFA). In certain aspects, a multispecific binding protein can further comprise or fused to an agent targeting a particular tumor. An exemplary agent can include pegaptanib, bevacizumab, ranibizumab, brolucizumab, aflibercept (e.g., to target VEGFA).

In certain aspects, a target protein can be an inflammatory protein. Inflammatory proteins are involved in the immune response and can contribute to chronic inflammation, autoimmune disorders, or tissue damage. A multispecific binding protein can be designed to target inflammatory proteins and help regulate the inflammatory cascade, suppress excessive immune responses, or modulate immune cell functions. By selectively binding and neutralizing inflammatory proteins, a multispecific binding protein of the disclosure can dampen inflammation and restore immune balance in various inflammatory conditions. Examples of inflammatory conditions include rheumatoid arthritis, dermatitis, and systemic lupus erythematosus (SLE). In certain aspects, a multispecific binding protein can comprise the variable domains of, be co-administered with, or fused to an agent targeting a proinflammatory protein of interest. An exemplary agent can include Eculizumab or Ravulizumab (e.g., to target complement component C5).

In certain aspects, a target protein can be an interleukin. In certain aspects, a multispecific binding protein can comprise the variable domain of, be co-administered with, or fused to an agent targeting an IL of interest. Interleukins (ILs) are a specific group of signaling molecules involved in immune responses and inflammation. A multispecific binding protein of the disclosure can be engineered to target specific ILs or their receptors thereby modulating their activity and downstream signaling. This approach can be applied to various immune-related disorders, such as autoimmune diseases, allergies, or inflammatory conditions. By interfering with IL signaling, a multispecific binding protein can regulate immune cell activation, cytokine production, or immune cell trafficking, providing a potential avenue for therapeutic intervention. An exemplary agent can include Siltuximab (e.g., to target IL-6), Mepolizumab or Reslizumab (e.g., to target IL-5), Secukinumab or Ixekizumab (e.g., to target IL-17A), Guselkumab, Tildrakizumab, or Risankizumab (e.g., to target the p19 subunit of IL-23), Rilonacept (e.g., to target IL-1A and IL-1B) Canakinumab (e.g., to target IL-1B), Ustekinumab (e.g., to target the p40 subunit of IL-12 and IL-23). In certain aspects, the interleukin can be IL-15, IL-4, IL-7, IL-9, and IL-21, or IL-2.

In certain aspects, a target protein can be a Wnt protein. Wnt proteins are a family of secreted signaling molecules that regulate cell proliferation, differentiation, and tissue development. Dysregulation of Wnt signaling is implicated in numerous diseases, including cancer, developmental disorders, and degenerative diseases. A multispecific binding protein targeting Wnt proteins can modulate their activity, block aberrant signaling pathways, or interfere with Wnt protein interactions. This approach offers potential therapeutic strategies for diseases driven by aberrant Wnt signaling. In certain aspects, a multispecific binding can comprise the variable domains, of, be co-administered with, or fused to an agent targeting an Wnt protein of interest. An exemplary agent can include Vantictumab (e.g., to target FZD1/2/5/7/8).

In certain aspects, a target protein can be a cytokine. In certain aspects, the cytokine can be member of the Transforming Growth Factor-beta (TGF-β). Members of the TGF-β family are a group of multifunctional cytokines involved in various cellular processes, including cell growth, differentiation, immune regulation, and tissue repair. Dysregulation of TGF-β signaling is associated with fibrosis, cancer progression, immune disorders, and other diseases. A multispecific binding protein designed to target TGF-β family members can modulate their signaling pathways, inhibit their effects on immune cells or stromal cells, or interfere with TGF-β ligand-receptor interactions. By modulating TGF-β signaling, a multispecific binding protein holds potential therapeutic value for a range of diseases associated with TGF-β dysregulation. In certain aspects, a TGF-β cytokine is a TGF-β1. In other aspects, the cytokine can be a member of the insulin-like growth factors (IGF). An exemplary IGF of the present disclosure includes IGF-1 and IGF-2. Other exemplary cytokines of the current disclosure include IgE and IgA. In certain aspects, a multispecific binding protein can comprise the variable domains of, be co-administered with, or fused to an agent targeting a cytokine of interest. An exemplary agent can include omalizumab (e.g., to target IgE).

In certain aspects, a target protein can be a Notch ligand. Notch ligands are cell surface proteins involved in cellular communication and tissue development. Dysregulation of Notch signaling is implicated in cancer, cardiovascular diseases, and neurodegenerative disorders. A multispecific binding protein of the disclosure can disrupt Notch signaling pathways, block ligand-receptor interactions, or modulate downstream gene expression. This approach offers potential therapeutic strategies for diseases driven by aberrant Notch signaling, with the goal of restoring normal cellular processes and tissue homeostasis.

In certain aspects, a target protein is expressed on a T-cell membrane (e.g., on the same IL-2Rβγ positive T-cell that is expressing the IL-2Rβ subunit, the IL-2Rγ subunit, or both IL-2Rβγ subunits). In other aspects, a target protein is expressed on activated T cells and/or Treg cells. In some aspects, the target protein comprises a membrane associated protein, including immune checkpoint proteins and receptors expressed on T cell surface.

In certain aspects, a target protein is an immune checkpoint protein and the second binding moiety comprises an agonist or an antagonist immune checkpoint modulator (e.g., an agonist or an antagonist immune checkpoint inhibitor). In some aspects, a second binding moiety comprises an agonist or an antagonistic antibody or antigen-binding fragment thereof against a receptor involved in immune modulation. In some aspects, a second binding moiety comprises an agonist or an antagonistic ISVD against a receptor involved in immune modulation.

In certain aspects, the immune checkpoint protein is a programmed cell death protein (“PD-1,” also known as “CD279”), a programmed cell death-ligand 1 (“PD-L1,” also known as “CD274” or “B7-H1”). In certain aspects, the second binding moiety is antagonistic modulator (i.e., an inhibitor) of an immune checkpoint protein.

Interaction of PD-L1 with its receptor PD1 on T cells delivers a signal that inhibits TCR-mediated activation of IL-2 production and T cell proliferation (e.g., antigen specific T cells in the lymph nodes) as well as simultaneously reducing apoptosis in regulatory T cells (e.g., anti-inflammatory, suppressive T cells). Dysregulation of the PD1/PD-L1 interaction and related signaling has been implicated in the pathogenesis of neoplastic disorders, inflammatory disorders, and autoimmune disorders.

In certain aspects, the second binding moiety blocks the interaction of a target protein with other proteins resulting in immune modulation. For example, in certain aspects, the second binding moiety blocks the interaction of the immune checkpoint protein with other proteins resulting in immune modulation. In certain aspects, the second binding moiety blocks the interaction between PD-1 and PD-1. PD-L1 is a membrane protein that forms a complex with its receptor PD1. Analytical measurement to determine whether the second binding moiety blocks the interaction between PD-1 and PD-L1 are known in the art, e.g., see PD-1:PD-L1 Cell-Based Inhibitor Screening Assay Kit (BPS Biosience; catalog #79377) or the methods described in Example 8. In certain aspects, a multispecific binding protein comprising: a) a first cell surface binding moiety that specifically binds to a CD122 (IL-2Rβ) subunit and/or a CD132 (IL-2Rγ) subunit, on the surface of an IL-2Rβγ positive cell, wherein the first cell surface binding moiety comprises an immunoglobulin domain; and b) a second binding moiety that is operatively linked to the first cell surface binding moiety and that specifically binds to a PD-L1. In certain aspects, specific binding of the multispecific binding protein to the IL-2Rβ subunit and/or the IL-2Rγ subunit facilitates internalization of the target protein bound to the multispecific binding protein into the IL-2Rβγ positive cell.

In certain aspects, the multispecific binding protein comprises at least three ISVDs,
wherein the at least three ISVDs comprise: a first ISVD that specifically binds to IL-2Rβ, a second ISVD that specifically binds IL-2Rγ, and a third ISVD that blocks PD1/PD-L1 interaction.

In certain aspects, a multispecific binding protein blocks the PD1/PD-L1 interaction as measured by TCR signaling in T cells expressing human PD-1 co-cultured with cells expressing PD-L1. In certain aspects, a multispecific binding protein blocks the PD1/PD-L1 interaction as measured in Jurkat T cells expressing human PD-1 and a reporter gene co-cultured with CHO-K1 cells expressing human PD-L1 and activating TCRs in an antigen independent manner. In certain aspects, the reporter gene is a luciferase reporter gene driven by a NFAT promoter. In certain aspects, a half-maximal inhibitory concentration (IC50 [M]) of the multispecific binding protein for blocking the PD1/PDL1 interaction as measured by TCR signaling in T cells expressing human PD-1 co-cultured with cells expressing PD-L1 is at least 5.0×10⁻⁹, 1.0×10⁻⁹, 5.0×10⁻¹⁰, 1.0×10⁻¹⁰, 5.0×10⁻¹¹, or 1.0×10⁻¹¹ M.

In certain aspects, the multispecific binding protein activates peripheral blood T cells. In certain aspects, the activation of the peripheral blood T cells is measured by cytokine production. In certain aspects, the cytokine is IFN-γ.

In certain aspects, the multispecific binding protein activates peripheral blood T cells in an antigen dependent manner. In certain aspects, the antigen dependent activation is measured within peripheral blood mononuclear cells (PBMCs). In certain aspects, a multispecific binding protein activates peripheral blood T cells as measured by IFNγ release in a tetanus toxoid recall assay. In certain aspects, a multispecific binding protein activates tetanus toxoid specific peripheral blood T cells as measured by IFNγ release.

In certain aspects, a multispecific binding protein comprises at least three ISVDs, wherein the at least three ISVDs comprise: a first ISVD that specifically binds to IL-2Rβ, a second ISVD that specifically binds IL-2Rγ, and a third ISVD that specifically binds to the target protein.

In certain aspects, a multispecific binding protein of the disclosure exhibits pH-dependent binding to a target protein. In certain aspects, a multispecific binding protein exhibits reduced binding at acidic pH. In certain aspects, the second binding moiety of a multispecific binding protein binds to a target protein with an affinity from about 100 pM to about 1 μM.

### Cell Internalization

One function of IL-2Rβγ is the uptake IL-2-bearing molecules or compositions and their internalization and transport to the lysosomes. For example, IL-2Rβγ can bind IL-2. IL-2Rβγ-mediated internalization of IL-2 requires the presence of the IL-2 receptor (i.e., requiring heterotrimerization of IL-2Rαβγ or heterodimerization of IL-2Rβγ) and is referred to herein as the IL-2 complex. Once IL-2 binds IL-2Rβγ, the IL-2 complex is internalized via endocytosis. IL-2Rβγ traffics with the IL-2 complex in early recycling compartments. Upon dissociation of the complex, IL-2Rα, if present, is recycled back to the cell surface membrane. Cendrowski et al. (2016), Cytokine Growth Factor Rev., vol. 32: 63-73. Differently, IL-2Rβγ subunits are found to traffic with IL-2 to the late endocytic compartments. Accordingly, along with IL-2, IL-2Rβγ subunits are ultimately degraded in the lysosome by lysosomal degradation. Hemar et al. (1995), J Cell Biol, vol. 129 (1): 55-64; Su et al. (2015), Sci Transl Med., vol. 7(311): 311ra170.

Therefore, multispecific binding proteins disclosed herein, which comprise an IL-2Rβ subunit, an IL-2Rγ subunit, or both IL-2Rβγ subunits targeting moiety, such as an IL-2Rβ subunit, an IL-2Rγ subunit, or both IL-2Rβγ subunits antibody or an antigen binding fragment thereof, can deliver and release a bound target protein to the endocytic compartment whereby an IL-2Rβ subunit, an IL-2Rγ subunit, or both IL-2Rβγ subunits, a target protein, and a multispecific binding protein dissociate.

After dissociation, in a pH-dependent manner the multispecific binding protein (e.g., an IL-2Rβ subunit, an IL-2Rγ subunit, or both IL-2Rβγ subunits antibody or an antigen binding fragment thereof) can be recycled back to the cell surface membrane whereby the multispecific binding protein (e.g., an IL-2Rβ subunit, an IL-2Rγ subunit, or both IL-2Rβγ subunits antibody or antigen binding fragment thereof or an IL-2Rβ subunit, an IL-2Rγ subunit, or both IL-2Rβγ subunits ISVD) is released back into the circulation. In one aspect, a multispecific binding protein is recycled back to the surface of the cell and released into the circulation where it can bind additional target protein.

Concomitantly, during dissociation, the IL-2Rβ subunit, the IL-2Rγ subunit, and/or the IL-2Rβγ heterodimer is not recycled back to the surface of the cell independent of the target protein. In some aspects, the IL-2Rβ subunit, the IL-2Rγ subunit, or the IL-2Rβγ dimer is present in a late endosome. In some aspects, the IL-2Rγ subunit, or the IL-2Rβγ dimer colocalizes with a rab-7 late endosomal marker. In some aspects, the IL-2Rβ subunit, the IL-2Rγ subunit, or the IL-2Rβγ dimer is degraded in the lysosome.

Like IL-2Rβγ subunits, the target protein continues along the late endocytic pathway for ultimate lysosomal degradation. In this way, IL-2Rβγ subunits bound to the multispecific binding protein of the disclosure can act as a shuttling mechanism for taking target proteins to the lysosomal degradation pathway.

Accordingly, in certain aspects, a multispecific binding protein of the disclosure can be internalized by an IL-2Rβγ positive cell. In certain aspects, the amount of multispecific binding protein internalized by the IL-2Rβγ positive cell is greater than the amount of a reference binding protein or polypeptide lacking the targeting moiety internalized by the cell. In certain aspects, the reference binding protein is encoded by an amino acid sequence set forth in SEQ ID NO: 59. The amino acid sequence set forth in SEQ ID NO: 59 is:

DVQLVESGGGVVQPGGSLRLSCAATGFTLDYYVIGWFRQAPGKEREGVAC
LSSNGDRINYADSVKGRFTISRDIAKNTVYLQMNSLRPEDTALYHCAAGT
STTVRDMCGIMYLYDYWAQGTLVTVSSGGGGSGGGSEVQLVESGGGVVQP
GGSLRLSCAATGFTLDYYVIGWFRQAPGKEREGVACLSSNGDRINYADSV
KGRFTISRDIAKNTVYLQMNSLRPEDTALYHCAAGTSTTVRDMCGIMYLY
DYWAQGTLVTVSSGGGGSGGGGSGGGGSGGGGSEVQLVESGGGVVQPGGS
LRLSCAASGFTFRSFGMSWVRQAPGKGPEWVSSISGSGSDTLYADSVKGR
FTISRDNSKNTLYLQMNSLRPEDTALYYCTIGGSLSRSSQGTLVTVSSA.

In certain aspects, the disclosure provides a multispecific binding protein comprising: a) a first cell surface binding moiety that specifically binds to an IL-2Rβ subunit, an IL-2Rγ subunit, or both IL-2Rβγ subunits on the surface of an IL-2Rβγ positive cell; and b) a second binding moiety that is operatively linked to the first cell surface binding moiety and that specifically binds to a target protein, wherein specific binding of the multispecific binding protein to the IL-2Rβγ positive cell facilitates internalization of the target protein bound to the multispecific binding protein.

In one aspect, the disclosure provides a multispecific binding protein comprising: a) a first cell surface binding moiety that specifically binds to an IL-2Rβ subunit, an IL-2Rγ subunit, or both IL-2Rβγ subunits on the surface of an IL-2Rβγ positive cell, wherein the first cell surface binding moiety comprises an immunoglobulin domain; and b) a second binding moiety that is operatively linked to the first cell surface binding moiety and that specifically binds to a target protein, wherein specific binding of the multispecific binding protein to the IL-2Rβ subunit, the IL-2Rγ subunit, or both IL-2Rβγ subunits facilitates internalization of the target protein bound to the multispecific binding protein into the IL-2Rβγ positive cell.

In certain aspects, the target protein traffics to lysosomes for degradation of the target protein. In certain aspects, an IL-2Rβ subunit, an IL-2Rγ subunit, or both IL-2Rβγ subunits is/are not recycled back to the surface while the multispecific binding protein is recycled back to the surface of the cell independent of the target protein. In certain aspects, an IL-2Rβ subunit, an IL-2Rγ subunit, or both IL-2Rβγ subunits multispecific targeting binding protein is recycled back to the surface of the cell where it can bind additional target protein for degradation.

In certain aspects, a multispecific binding protein of the disclosure exhibits increased degradation of the target protein compared to a reference binding protein. In certain aspects, the reference binding protein does not comprise the first cell surface binding moiety that specifically binds to the IL-2Rβ subunit, the IL-2Rγ subunit, or both IL-2Rβγ subunits, but is otherwise identical to the multispecific binding protein. In certain aspects, a multispecific binding protein exhibits increased degradation of the target protein by at least 2, 3, 4, 5, 10, 20, 30, 40, 50, 60, 70, 80, 90, or 100 percent compared to the reference binding protein. In certain aspects, a multispecific binding protein degrades the target protein at least 2, 3, 4, 5, 10, 24, 48, or 72 hours faster compared to the reference binding protein. In certain aspects, the reference binding protein sequence is set forth in SEQ ID NO: 59.

In certain aspects, the multispecific binding protein's maximal amount of degradation (DMax) of the target protein is 55, 60, 65, 75, 80, 85, 90, 95, or 100 percent.

The cell internalization of a multispecific binding protein of the disclosure can deplete a target protein from the circulation and/or a specific tissue. In one aspect, the method of depleting a target protein comprises administering to a subject an effective amount of the disclosed multispecific binding protein or a pharmaceutical composition comprising the same.

In certain aspects, upon administration of a multispecific binding protein described herein, the target protein is selectively depleted from the circulation or from a target tissue of the subject. In some aspects, administering the multispecific binding protein results in 10%, 20%, 30%, 40%, 50%, 75%, or 90% depletion of the target protein from the target tissue or circulation of the subject.

### Disease Indications

By exploiting the IL-2Rβγ shuttling mechanism, the methods enable the efficient and selective degradation of various target proteins, including pathogenic proteins, proteins secreted by tumors, autoantibodies, inflammatory proteins, interleukins, and signaling molecules. The disclosed methods can be used for the development of targeted therapies with precise control over the degradation of specific molecules for the treatment of diseases such as autoimmune disorders, cancer, and inflammatory conditions.

By facilitating the degradation of target proteins, the multispecific binding proteins of the disclosure are useful, inter alia, for the treatment, prevention and/or amelioration of any disease or disorder associated with or mediated by target protein expression, signaling, or activity, or treatable by IL-2Rβγ-mediated degradation of the target protein within the lysosome. For example, the present disclosure provides methods for treating autoimmune disease, cancer (tumor growth inhibition), chronic viral infections, and other diseases by administering the multispecific binding proteins described herein to a patient in need of such treatment.

The multispecific binding proteins of the present disclosure are useful for the treatment, prevention, and/or amelioration of disease or disorder or condition such as autoimmune disease, a viral infection, or cancer and/or for ameliorating at least one symptom associated with such disease, disorder, or condition. In the context of the methods of treatment described herein, the multispecific binding proteins can be administered as a monotherapy (i.e., as the only therapeutic agent) or in combination with one or more additional therapeutic agents.

In certain aspects, a multispecific binding protein described herein is useful for treating subjects suffering from a disease selected from a group consisting of: cancer, autoimmune disease, inflammatory disorder, infectious disease, and neurodegenerative disorder.

### Additional Terms

### Kabat, EU, and AbM Numbering

Amino acid positions in a heavy chain constant region, including amino acid positions in the CH1, hinge, CH2, CH3, and CL domains, can be numbered according to the Kabat index numbering system (see Kabat et al., in “Sequences of Proteins of Immunological Interest”, U.S. Dept. Health and Human Services, 5th edition, 1991). Alternatively, antibody amino acid positions can be numbered according to the EU index numbering system (see Kabat et al.).

In the CDR determination according to Kabat, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

CDR sequences can also be determined according to the AbM numbering as described in Kontermann and Dubel (2010), Antibody Engineering, vol. 2, Springer Verlag Heidelberg Berlin, Martin, Chapter 3, pp. 33-51. According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

### Specifically Binds

The term “specifically binds” as used herein, refers to the ability of an antibody or an antigen-binding fragment thereof to bind to an antigen with a dissociation constant (K_{D}) of at most about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or less, and/or to bind to an antigen with an affinity that is at least two-fold greater than its affinity for a nonspecific antigen. Specific binding of an antibody can be to a target antigen through the CDR sequences. An antibody can also specifically bind to FcRs, such as FcRn or FcγRIIIa through the Fc region.

### Dissociation Constant (K_{D})

The dissociation constant (K_{D}) of a binding protein can be determined, for example, by surface plasmon resonance. Generally, surface plasmon resonance analysis measures real-time binding interactions between ligand (a target antigen on a biosensor matrix) and analyte (a binding protein in solution) by surface plasmon resonance (SPR) using the Biacore system (Cytiva Life Sciences, Marlborough, MA) or Carterra LSA platform (Carterra, Salt Lake City, UT). Surface plasmon analysis can also be performed by immobilizing the analyte (binding protein on a biosensor matrix) and presenting the ligand (target antigen). The term “K_{D}” as used herein refers to the dissociation constant of the interaction between a particular binding protein and a target antigen.

### Degradation Constant (DC₅₀)

The half-maximal degradation constant (DC₅₀) is the concentration to achieve half-maximal degradation of the target protein. DC₅₀ is calculated as the abscissa of the half-maximal degradation point. In some aspects, the multispecific binding protein's DC₅₀ concentration of the target protein is 0.2, 0.3, 0.4, 0.5, 0.6, 0.7. 0.8, 0.9 nM. In some aspects, the multispecific binding protein's DC₅₀ concentration of the target protein is 20, 25, 30, 35, 40, 45, 50, 55, 60, 65, 75, or 80 percent. Degradation maximum (D_{Max}) is the maximal percentage of protein degraded where 100% indicates no target protein remains. In some aspects, the multispecific binding protein's maximal amount of degradation (DMax) of the target protein is 55, 60, 65, 75, 80, 85, 90, 95, or 100 percent.

### Half Maximal Inhibitory Concentration (IC50)

The half maximal inhibitory concentration (IC50) refers to the concentration of a multispecific binding protein which induces an inhibitory response halfway between the baseline and maximum after a specified exposure time. In the present context, it is used as a measure the antagonistic potency of a multispecific binding protein or its underlying binding elements, e.g., a first cell surface binding moiety or a second binding moiety that specifically binds to the target protein. The IC50 of a graded dose response curve represents the concentration of a compound where 50% of its maximal inhibitory effect is observed. Concentration is preferably expressed in molar units.

### Half Maximal Effective Concentration (EC50)

The half maximal effective concentration (EC50) refers to the concentration of a multispecific binding protein which induces a response halfway between the baseline and maximum after a specified exposure time. In the present context, it is used as a measure the agonist potency of a multispecific binding protein or its underlying binding elements, e.g., a first cell surface binding moiety or a second binding moiety that specifically binds to the target protein. The EC50 of a graded dose response curve represents the concentration of a compound where 50% of its maximal effect is observed. Concentration can be expressed in molar units.

### Valency

As used herein the term “valency” refers to the number of potential target binding sites in a multispecific binding protein. Each target binding site specifically binds one target molecule or specific site on a target molecule. When a polypeptide comprises more than one target binding site, each target binding site can specifically bind the same or different molecules (e.g., can bind to more than one target protein, or different epitopes on the same target protein). A subject multispecific binding protein has at least one binding site (e.g., 1, 2, 3, or more) specific for an IL-2Rβ subunit, an IL-2Rγ subunit, or both IL-2Rβγ subunits. A subject a multispecific binding protein has at least one binding site (e.g., 1, 2, 3, 4, or more) for a target protein.

### Specificity

The term “specificity” refers to the ability to specifically bind (e.g., immunoreact with) a given target antigen (e.g., an IL-2Rβ subunit, an IL-2Rγ subunit, or both IL-2Rβγ subunits, or a target protein). A subject multispecific binding protein contains two or more binding sites (e.g., 2, 3, 4, 5, or more) which specifically bind the same or different binding sites. In certain aspects, a subject multispecific binding protein is specific for two different (e.g., non-overlapping) target binding sites.

In certain aspects, the multispecific binding proteins of the disclosure employ one or more of an IL-2Rβ subunit, an IL-2Rγ subunit, or both IL-2Rβγ subunits binding moieties which bind to a human IL-2Rβ subunit, IL-2Rγ subunit, or both IL-2Rβγ subunits, but not to an IL-2Rβ subunit, an IL-2Rγ subunit, or both IL-2Rβγ subunits from other species. Alternatively, the multispecific binding protein employ an IL-2Rβ subunit, an IL-2Rγ subunit, or both IL-2Rβγ subunits binding moiety which binds to human and non-human species. For example, the multispecific binding protein can bind to a human IL-2Rβ subunit, IL-2Rγ subunit, or both IL-2Rβγ subunits and can bind or not bind, as the case can be, to one or more of mouse, rat, guinea pig, hamster, gerbil, pig, cat, dog, rabbit, goat, sheep, cow, horse, camel, cynomolgus, marmoset, rhesus or chimpanzee IL-2Rβ subunit, IL-2Rγ subunit, or both IL-2Rβγ subunits. In certain aspects, the multispecific binding protein can bind to a human IL-2Rβ subunit, IL-2Rγ subunit, or both IL-2Rβγ subunits, but do not bind to rat and mouse IL-2Rβ subunits, IL-2Rγ subunits, or both IL-2Rβγ subunits. In other aspects, the multispecific binding protein binds to human IL-2Rβ subunit, IL-2Rγ subunit, or both IL-2Rβγ subunits, and to rat and mouse IL-2Rβ subunits, IL-2Rγ subunits, or both IL-2Rβγ subunits, with similar binding affinities.

### About or Approximately

The term “about” or “approximately” means within about 20%, such as within about 10%, within about 5%, or within about 1% or less of a given value or range.

As used herein, “administer” or “administration” refers to the act of injecting or otherwise physically delivering a substance as it exists outside the body (e.g., a multispecific binding protein provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and can be continued chronically to defer or reduce the appearance or magnitude of disease-associated symptoms.

### Composition

As used herein, the term “composition” is intended to encompass a product containing the specified ingredients (e.g., a multispecific binding protein composition provided herein) in, optionally, the specified amounts, as well as any product which results, directly or indirectly, from combination of the specified ingredients in, optionally, the specified amounts.

### Effective Amount

“Effective amount” means the amount of active pharmaceutical agent (e.g., a multispecific binding protein of the present disclosure) sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

### Subject or Patient

As used herein, the terms “subject” and “patient” are used interchangeably. As used herein, a subject can be a mammal, such as a non-primate (e.g., cows, pigs, horses, cats, dogs, rats, etc.) or a primate (e.g., monkey and human). In certain aspects, the term “subject,” as used herein, refers to a vertebrate, such as a mammal. Mammals include, without limitation, humans, non-human primates, wild animals, feral animals, farm animals, sport animals, and pets.

### Therapy and Pharmaceutical Compositions

As used herein, the term “therapy” refers to any protocol, method and/or agent that can be used in the prevention, management, treatment and/or amelioration of a disease or a symptom related thereto. In some aspects, the term “therapy” refers to any protocol, method and/or agent that can be used in the modulation or depletion of a target protein from the circulation of a subject. In some aspects, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other aspects, the terms “therapies” and “therapy” refer to a biological therapy, supportive therapy, and/or other therapies useful in the modulation of an immune response to an inflammatory or autoimmune diseases in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as the administration of a multispecific binding protein provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

Multispecific binding proteins of the current disclosure are useful in a number of different applications. In certain aspects, a multispecific binding protein disclosed herein is effective in reducing the concentration of or eliminating a target protein in the circulation of a subject. In certain aspects, a multispecific binding protein can reduce inflammatory symptoms. In certain aspects, a multispecific binding protein can reduce tumor size. Methods of preparing and administering a multispecific binding protein of the current disclosure to a subject are well known to or are readily determined by those skilled in the art. The route of administration of a multispecific binding protein of the current disclosure can be oral, parenteral, by inhalation, or topical, or other suitable method. The term parenteral as used herein includes intravenous, intraarterial, intraperitoneal, intramuscular, subcutaneous, rectal, or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection can comprise a buffer (e.g., acetate, phosphate or citrate buffer), a surfactant (e.g., polysorbate), optionally a stabilizer agent (e.g., human albumin), etc. However, in other methods compatible with the teachings herein, the multispecific binding protein can be delivered directly to the site of the adverse tissue thereby increasing the exposure of the diseased tissue to the therapeutic agent. In certain aspects, a pharmaceutical composition comprises a multispecific binding protein described herein and a pharmaceutically acceptable carrier or diluent. In certain aspects, a method of depleting a target protein comprises administering to a subject an effective amount of a multispecific binding protein of the disclosure or the pharmaceutical composition comprising the multispecific binding protein. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M, e.g., 0.05M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will typically be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

In many cases, isotonic agents will be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin. In any case, sterile injectable solutions can be prepared by incorporating an active compound (e.g., a multispecific binding protein by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Such articles of manufacture will typically have labels or package inserts indicating that the associated compositions are useful for treating a subject suffering from, or predisposed to autoimmune or neoplastic disorders.

Effective doses of multispecific binding protein compositions of the present disclosure, for the treatment of the conditions described above vary depending upon many different factors, including means of administration, target site, physiological state of the patient, whether the patient is human or another animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

Multispecific binding proteins of the current disclosure can be administered on multiple occasions. Intervals between single dosages can be weekly, monthly or yearly. Intervals can also be irregular as indicated by measuring blood levels of a target protein in the patient. Alternatively, multispecific binding protein can be administered as a sustained release formulation, in which case less frequent administration is required. For multispecific binding protein, dosage and frequency vary depending on the half-life of the multispecific binding protein in the patient.

The dosage and frequency of administration can vary depending on whether the treatment is prophylactic or therapeutic. In prophylactic applications, compositions containing the present a multispecific binding protein or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from about 0.1 to about 25 mg per dose, especially about 0.5 to about 2.5 mg per dose. A relatively low dosage is administered at relatively infrequent intervals over a long period of time. Some patients continue to receive treatment for the rest of their lives. In therapeutic applications, a relatively high dosage (e.g., from about 1 to 400 mg/kg of a multispecific binding protein per dose) at relatively short intervals is sometimes required until progression of the disease is reduced or terminated, or until the patient shows partial or complete amelioration of disease symptoms. Thereafter, the patient can be administered a prophylactic regime.

A pharmaceutical composition in accordance with the present disclosure can comprise a pharmaceutically acceptable, non-toxic, sterile carrier such as physiological saline, nontoxic buffers, preservatives and the like. For the purposes of the instant application, a pharmaceutically effective amount of a multispecific binding protein disclosed herein, shall be held to mean an amount sufficient to achieve effective binding to a target protein and to achieve a benefit, e.g., to ameliorate symptoms of a disease or disorder.

### Determining Multispecific Binding Protein Binding and Specificity

The binding affinity of a first cell surface binding moiety comprising an IL-2Rβ subunit, IL-2Rγ subunit, or both IL-2Rβγ subunits antibody or an antigen binding fragment thereof that binds to IL-2Rβγ, or a second binding moiety that binds to the target protein can each be assessed using, e.g., surface plasmon resonance, ELISA, or other suitable method (see Shields et al. (2001) J. Biol. Chem., 276:6591-6604.)

In some aspects, the binding constant K_{D} of a multispecific binding protein for an IL-2Rβ subunit, an IL-2Rγ subunit, or both IL-2Rβγ subunits can be above that of the wild-type control by 1, 2, 3, 4, 5, 6, 7, 8, 9, 10, 11, 12, 13, 14, 15, 16, 17, 18, 19, or 20-fold or higher. The wild-type control can be an IL-2Rβ subunit, an IL-2Rγ subunit, or both IL-2Rβγ subunits antibody or an antigen binding fragment thereof. The binding constant K_{D} of a multispecific binding protein for an IL-2Rβ subunit, an IL-2Rγ subunit, or both IL-2Rβγ subunits or a target protein can be substantially the same (i.e., ±50%) as the wild-type control or above it.

In some aspects, the binding constant K_{D} of a multispecific binding protein of the disclosure for a target protein can be substantially the same (i.e., ±50%) as the wild-type control or below it.

In some aspects, the binding constant K_{D} of a multispecific binding protein for a target protein can be above that of the wild-type control by 1, 2, 3, 4, 5, 6, 7, 8, 9, 10, 11, 12, 13, 14, 15, 16, 17, 18, 19, or 20-fold or higher. The wild-type control can be an antibody or an antigen binding fragment thereof that binds to the target protein. The binding constant Kₚ of a multispecific binding protein for the target protein can be substantially the same (i.e., ±50%) as the wild-type control or above it.

The binding specificity of a multispecific binding protein to an IL-2Rβ subunit, an IL-2Rγ subunit, or both IL-2Rβγ subunits or a target protein can be determined by, e.g., flow cytometry, western blotting, or another suitable method. In some aspects, a multispecific binding protein is directed against or specifically binds an IL-2Rβ subunit, an IL-2Rγ subunit, or both IL-2Rβγ subunits. In some aspects, a multispecific binding protein is directed against or specifically binds a target protein. A multispecific binding protein can be either specific to a human an IL-2Rβ subunit, an IL-2Rγ subunit, or both IL-2Rβγ subunits or can cross-react with corresponding targets from other species. A multispecific binding protein can be either specific to a human target protein or can cross-react with corresponding targets from other species.

In some aspects, the binding constant K_{D} of a multispecific binding protein comprising an IL-2Rβ subunit, an IL-2Rγ subunit, or both IL-2Rβγ subunits antibody or an antigen binding fragment thereof can be above that of the wild-type control by 1, 2, 3, 4, 5, 6, 7, 8, 9, 10, 11, 12, 13, 14, 15, 16, 17, 18, 19, or 20-fold or higher. The wild-type control can either be an IL-2Rβ subunit, an IL-2Rγ subunit, or both IL-2Rβγ subunits antibody or an antigen binding fragment or an antibody or an antigen binding fragment that binds the target protein. The binding constant K_{D} of a multispecific binding protein for an IL-2Rβ subunit, an IL-2Rγ subunit, or both IL-2Rβγ subunits or a target protein can be substantially the same (i.e., 50%) as the wild-type control or above it.

The binding specificity of a multispecific binding protein to an IL-2Rβ subunit, IL-2Rγ subunit, or both IL-2Rβγ subunits or to a target protein can be determined by, e.g., flow cytometry, western blotting, or another suitable method. In some aspects, a multispecific binding protein specifically binds to an IL-2Rβ subunit, an IL-2Rγ subunit, or both IL-2Rβγ subunits and/or a target protein.

In some aspects, certain pharmacokinetic parameters of a multispecific binding protein of the disclosure are same or better that those of wild-type control. For example, in some aspects, elimination half-life (t_{1/2}) and/or the area under the concentration curve (AUC) can be substantially the same (i.e., 50%) as the wild-type control or above it. Pharmacokinetic parameters can be measured in humans or using an appropriate animal model (See, e.g., Shargel et al. (1995) Applied Biopharmaceutics and Pharmacokinetics, 4th ed., McGraw-Hill/Appleton.)

### Polynucleotides

In one aspect, polynucleotides (i.e., nucleic acid molecules) encoding a multispecific binding protein described herein or variants thereof are provided. A polynucleotide variant as used herein is about 50, 75, 80, 85, 90, 93, 95, 98, 99% or more identical to a polynucleotide that encodes a multispecific binding protein described herein.

In one aspect, nucleic acid molecules encode an amino acid sequence set forth in SEQ ID NO: 59-63.

Methods of making a multispecific binding protein comprising expressing these polynucleotides are also provided. Polynucleotides encoding a multispecific binding protein or variants thereof disclosed herein are typically inserted in an expression vector for introduction into host cells that can be used to produce the desired quantity of the claimed multispecific binding protein. Accordingly, in certain aspects, the disclosure provides expression vectors comprising polynucleotides disclosed herein and host cells comprising these vectors and polynucleotides.

In some aspects, nucleic acid molecules encode an amino acid sequence for a) a first cell surface binding moiety that specifically binds to an IL-2Rβ subunit, an IL-2Rγ subunit, or both IL-2Rβγ subunits on the surface of an IL-2Rβγ positive cell; and b) a second binding moiety that is operatively linked to the first cell surface binding moiety and that specifically binds to a target protein.

### Expression Vector and Host Cell

The term “vector” or “expression vector” is used herein for the purposes of the specification and claims, to mean vectors used in accordance with the present disclosure as a vehicle for introducing into and expressing the polynucleotide sequence encoding a multispecific binding protein polypeptide in a cell. As known to those skilled in the art, such vectors can easily be selected from the group consisting of plasmids, phages, viruses and retroviruses. In general, vectors compatible with the instant disclosure will comprise a selection marker, appropriate restriction sites to facilitate cloning of the desired gene and the ability to enter and/or replicate in eukaryotic or prokaryotic cells.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

One or more genes encoding a multispecific binding protein can also be expressed in non-mammalian cells such as bacteria, yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides can become part of inclusion bodies. The polypeptides can be isolated, purified, and then assembled into functional molecules.

In addition to prokaryotes, eukaryotic cells can also be used. Saccharomyces cerevisiae, or common baker's yeast, is the most commonly used, although a number of other strains are commonly available.

In some aspects, a vector comprises nucleic acid molecules encoding an amino acid sequence for a) a first cell surface binding moiety that specifically binds to an IL-2Rβ subunit, an IL-2Rγ subunit, or both IL-2Rβγ subunits on the surface of an IL-2Rβγ positive cell; and b) a second binding moiety that is operatively linked to the first cell surface binding moiety and that specifically binds to a target protein.

In some aspects, at least two vectors comprise nucleic acid molecules encoding an amino acid sequence for a) a first cell surface binding moiety that specifically binds to an IL-2Rβ subunit, an IL-2Rγ subunit, or both IL-2Rβγ subunits on the surface of an IL-2Rβγ positive cell; and b) a second binding moiety that is operatively linked to the first cell surface binding moiety and that specifically binds to a target protein.

In some aspects, two vectors comprise polynucleotides encoding a multispecific binding protein of the disclosure. In some aspects, the first vector comprises a nucleic acid molecule encoding an amino acid sequence for a) a first cell surface binding moiety that specifically binds to an IL-2Rβ subunit, an IL-2Rγ subunit, or both IL-2Rβγ subunits on the surface of an IL-2Rβγ positive cell. In some aspects, the second vector comprises a polynucleotide encoding a second binding moiety that is operatively linked to the first cell surface binding moiety and that specifically binds to a target protein. In some aspects, two vectors comprise a multispecific binding protein of the disclosure.

In some aspects, a cell comprises a vector comprising a nucleic acid molecule encoding an amino acid sequence for a) a first cell surface binding moiety that specifically binds to an IL-2Rβ subunit, an IL-2Rγ subunit, or both IL-2Rβγ subunits on the surface of an IL-2Rβγ positive cell; and b) a second binding moiety that is operatively linked to the first cell surface binding moiety and that specifically binds to a target protein.

In some aspects, a cell comprises two vectors comprising nucleic acid molecules encoding an amino acid sequence for a) a first cell surface binding moiety that specifically binds to an IL-2Rβ subunit, an IL-2Rγ subunit, or both IL-2Rβγ subunits on the surface of an IL-2Rβγ positive cell; and b) a second binding moiety that is operatively linked to the first cell surface binding moiety and that specifically binds to a target protein.

In some aspects, a cell comprises at least two vectors comprising nucleic acid molecules encoding an amino acid sequence for a) a first cell surface binding moiety that specifically binds to an IL-2Rβ subunit, an IL-2Rγ subunit, or both IL-2Rβγ subunits on the surface of an IL-2Rβγ positive cell; and b) a second binding moiety that is operatively linked to the first cell surface binding moiety and that specifically binds to a target protein.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicant reserves the right to physically incorporate into this application any and all materials and information from any such articles, patents, patent applications, or other physical and electronic documents.

While the present disclosure has been described with reference to the specific aspects thereof, it should be understood by those skilled in the art that various changes can be made and equivalents can be substituted without departing from the true spirit and scope of the application. It will be readily apparent to those skilled in the art that other suitable modifications and adaptations of the methods described herein can be made using suitable equivalents without departing from the scope of the aspects disclosed herein. In addition, many modifications can be made to adapt a particular situation, material, composition of matter, process, process step or steps, to the objective, spirit and scope of the present disclosure. All such modifications are intended to be within the scope of the claims appended hereto. Having now described certain aspects in detail, the same will be more clearly understood by reference to the following examples, which are included for purposes of illustration only and are not intended to be limiting.

The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. Any single term, single element, single phrase, group of terms, group of phrases, or group of elements described herein can each be specifically excluded from the claims.

### EXAMPLES

The present disclosure is further illustrated by the following examples which should not be construed as further limiting. The contents of the Sequence Listing, figures and all references, patents, and published patent applications cited throughout this application are expressly incorporated herein by reference.

### Example 1: Generation of the ISVD Monovalent Constructs Targeting IL-2Rβ and/or IL-2Rγ

### Introduction

This Example summarizes the design and construction of a multispecific binding protein (e.g., a multispecific binding protein comprising several ISVDs) that can specifically bind to IL-2Rβ and/or IL-2Rγ at the cell surface of an IL-2Rβγ positive cell.

### Methods

After approval of the Ethical Committee (EC2018 #1), 3 llamas were immunized with DNA encoding for IL2Rβγ receptor according to standard protocols. From blood samples collected from each animal, PBMCs were prepared using Ficoll-Hypaque according to the manufacturer's instructions (Amersham Biosciences, Piscataway, NJ, USA). For each immunized llama, libraries were constructed by pooling the total RNA isolated from these blood samples. In short, RNA extracted from PBMC was used as starting material for RT-PCR to amplify V_{HH} encoding gene fragments. The PCR-amplified V_{HH} repertoire was cloned via specific restriction sites into a vector designed to facilitate phage display of the V_{HH} library. The vector was derived from pUC119. In frame with the V_{HH} coding sequence, the vector encodes a C-terminal 3×FLAG and His6 (SEQ ID NO: 470) tag. Phages were prepared according to standard protocols described in e.g., WO 2004/041865, WO 2004/041863, WO 2004/062551, and WO 2005/044858A1 which are herein incorporated by reference in their entirety. V_{HH} repertoires obtained from all llamas and cloned as phage library were used in different selection strategies, applying a multiplicity of selection conditions. IL-2Rβ or IL-2Rγ antigen preparations were presented at multiple concentrations. After 2 h incubation with the phage libraries, followed by extensive washing, bound phages were eluted with trypsin (1 mg/mL) for 15 minutes. The trypsin protease activity was immediately neutralized by applying 0.8 mM protease inhibitor ABSF. As control, selections without antigen were performed in parallel. Phage outputs were used to infect E. coli for analysis of individual V_{HH} clones. Single colonies were picked from the agar plates and grown in 1 mL 96-deep-well plates. LacZ-controlled V_{HH} expression was induced by addition of IPTG (1 mM final) in the absence of glucose. Periplasmic extracts were prepared according to standard protocols described in e.g., WO 2003/035694, WO 2004/041865, WO 2004/041863, and WO 2004/062551, which are herein incorporated in their entirety.

### Results

The DNA sequence of the positive clones was determined. Table 4 are amino acid sequences of framework (FR) and CDR regions of the ISVDs according to Abm CDR definition. Table 4B are amino acid sequences of framework (FR) and CDR regions of the ISVDs according to Kabat CDR definition.

TABLE 4
Sequences for CDRs and framework (FR) regions, using Abm definition
(“ID” refers to the SEQ ID No. attributed to each sequence).

| | | Sequence | |
|---|---|---|---|
| ID | ISVD | Type | Sequence |
| 104 | A0291159F01 | FR1 | EVQLVESGGGLVQAGDSTTISCVAS |
| 97 | A0291031G03 | FR1 | EVQLVESGGGLVQAGGSLRLSCVAS |
| 330 | A0291142F04 | FR1 | EVQLVESGGGWVQPGGSLRLSCSPS |
| 337 | A0291033D02 | FR1 | EVQLVESGGGLVQAGGSLRLSCAAS |
| 121 | A0291159F01 | CDR1 | GGSFATYAFG |
| 114 | A0291031G03 | CDR1 | GRTFSATNMG |
| 331 | A0291142F04 | CDR1 | ERLSSIDIMG |
| 338 | A0291033D02 | CDR1 | GRSFSSYYMG |
| 138 | A0291159F01 | FR2 | WFRQAPGKEREFVA |
| 131 | A0291031G03 | FR2 | WFRQAPGKEREFVG |
| 332 | A0291142F04 | FR2 | WYRQTPGKEREWVA |
| 339 | A0291033D02 | FR2 | WFRQAPGKEREFVA |
| 155 | A0291159F01 | CDR2 | TISQRGSTSY |
| 148 | A0291031G03 | CDR2 | IISESGRNTD |
| 334 | A0291142F04 | CDR2 | TITGDDTTH |
| 340 | A0291033D02 | CDR2 | GINWSGRSTH |
| 172 | A0291159F01 | FR3 | YSDSVEGRFTISKDNAKSTVYLQMNSLQPEDTAVYYCAA |
| 165 | A0291031G03 | FR3 | YADSVKGRFTITRDNAKSTVYLQMNNLKPEDTAVYYCAL |
| 335 | A0291142F04 | FR3 | YENSVKGRFTISRDTAKNMVYLQMNSLEPEDTAVYYCNA |
| 341 | A0291033D02 | FR3 | YADSVKGRFTISRDNAKNTVYLQMNSLKPEDRAAYYCAA |
| 189 | A0291159F01 | CDR3 | RYYGVDYRSTSYDF |
| 182 | A0291031G03 | CDR3 | GRDYFATDTRQYNY |
| 336 | A0291142F04 | CDR3 | GILPREEY |
| 342 | A0291033D02 | CDR3 | DSAPSTRIGVRSLWDDYDY |
| 435 | A0291159F01 | FR4 | WGQGTLVTVSS |
| 436 | A0291031G03 | FR4 | WGLGTLVTVSS |
| 437 | A0291142F04 | FR4 | WGQGTLVTVSS |
| 438 | A0291033D02 | FR4 | WGQGTLVTVSS |

TABLE 4B
Sequences for CDRs and framework (FR) regions, using Kabat definition
(“ID” refers to the SEQ ID No. attributed to each sequence).

| | | Sequence | |
|---|---|---|---|
| ID | ISVD | Type | Sequence |
| 439 | A0291159F01 | FR1 | EVQLVESGGGLVQAGDSTTISCVASGGSFA |
| 440 | A0291031G03 | FR1 | EVQLVESGGGLVQAGGSLRLSCVASGRTFS |
| 441 | A0291142F04 | FR1 | EVQLVESGGGWVQPGGSLRLSCSPSERLSS |
| 442 | A0291033D02 | FR1 | EVQLVESGGGLVQAGGSLRLSCAASGRSFS |
| 443 | A0291159F01 | CDR1 | TYAFG |
| 444 | A0291031G03 | CDR1 | ATNMG |
| 445 | A0291142F04 | CDR1 | IDIMG |
| 446 | A0291033D02 | CDR1 | SYYMG |
| 447 | A0291159F01 | FR2 | WFRQAPGKEREFVA |
| 448 | A0291031G03 | FR2 | WFRQAPGKEREFVG |
| 449 | A0291142F04 | FR2 | WYRQTPGKEREWVA |
| 450 | A0291033D02 | FR2 | WFRQAPGKEREFVA |
| 451 | A0291159F01 | CDR2 | TISQRGSTSYYSDSVEG |
| 452 | A0291031G03 | CDR2 | IISESGRNTDYADSVKG |
| 453 | A0291142F04 | CDR2 | TITGDDTTHYENSVKG |
| 454 | A0291033D02 | CDR2 | GINWSGRSTHYADSVKG |
| 455 | A0291159F01 | FR3 | RFTISKDNAKSTVYLQMNSLQPEDTAVYYCAA |
| 456 | A0291031G03 | FR3 | RFTITRDNAKSTVYLQMNNLKPEDTAVYYCAL |
| 457 | A0291142F04 | FR3 | RFTISRDTAKNMVYLQMNSLEPEDTAVYYCNA |
| 458 | A0291033D02 | FR3 | RFTISRDNAKNTVYLQMNSLKPEDRAAYYCAA |
| 459 | A0291159F01 | CDR3 | RYYGVDYRSTSYDF |
| 460 | A0291031G03 | CDR3 | GRDYFATDTRQYNY |
| 461 | A0291142F04 | CDR3 | GILPREEY |
| 462 | A0291033D02 | CDR3 | DSAPSTRIGVRSLWDDYDY |
| 463 | A0291159F01 | FR4 | WGQGTLVTVSS |
| 464 | A0291031G03 | FR4 | WGLGTLVTVSS |
| 465 | A0291142F04 | FR4 | WGQGTLVTVSS |
| 466 | A0291033D02 | FR4 | WGQGTLVTVSS |

The amino acid sequences of the anti-IL-2Rβ3 and anti-IL-2Rγ ISVDs are presented in Table 5 below and as set forth in SEQ ID NOs: 354-360.

TABLE 5
Amino acid sequences of individual anti-IL2βR and anti-IL2γR ISVDs (“ID”
refers to the SEQ ID NO. attributed to each sequence).

| ISVD | ID | Sequence |
|---|---|---|
| A0291159F01 | 354 | EVQLVESGGGLVQAGDSTTISCVASGGSFATYAFGWFRQAPGKEREFV |
| (anti-IL2Rγ) | | ATISQRGSTSYYSDSVEGRFTISKDNAKSTVYLQMNSLQPEDTAVYYC |
| | | AARYYGVDYRSTSYDFWGQGTLVTVSS |
| A0291031G03 | 347 | EVQLVESGGGLVQAGGSLRLSCVASGRTFSATNMGWFRQAPGKEREFV |
| (anti-IL2Rβ) | | GIISESGRNTDYADSVKGRFTITRDNAKSTVYLQMNNLKPEDTAVYYC |
| | | ALGRDYFATDTRQYNYWGLGTLVTVSS |
| A0291142F04 | 359 | EVQLVESGGGWVQPGGSLRLSCSPSERLSSIDIMGWYRQTPGKEREWV |
| (anti-IL2Rγ) | | ATITGDDTTHYENSVKGRFTISRDTAKNMVYLQMNSLEPEDTAVYYCN |
| | | AGILPREEYWGQGTLVTVSS |
| A0291033D02 | 360 | EVQLVESGGGLVQAGGSLRLSCAASGRSFSSYYMGWFRQAPGKEREFV |
| (anti-IL2β) | | AGINWSGRSTHYADSVKGRFTISRDNAKNTVYLQMNSLKPEDRAAYYC |
| | | AADSAPSTRIGVRSLWDDYDYWGQGTLVTVSS |

The SEQ ID NOs for the CDR sequences referred to above in Table 4 are based on the CDR definition according to the AbM definition. It is noted that the CDR sequences defined according to the Kabat definition, Chotia definition or IMGT definition can likewise be used. Accordingly, the ISVDs provided by the present technology, specifically binding to IL2Rγ or IL2Rβ3 as described above using the AbM definition, can be also described using the Kabat definition, Chotia definition or IMGT definition.

In one embodiment, the ISVD comprise CDRs (Kabat definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360.

In one embodiment, the ISVD comprise CDRs (Chothia definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360.

In one embodiment, the ISVD comprise CDRs (IMGT definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360.

### Conclusion

This example displays the construction of novel IVSDs that target either to IL-2Rβ or IL-2Rγ subunits of IL-2R.

### Example 2: Preparation of Multivalent Anti-IL-2Rβγ ISVD Constructs as Well as Engineering Additional Domains

### Introduction

This example demonstrates the design strategy of engineering multivalent anti-IL-2Rβγ ISVD constructs including the incorporation of other ISVDs into the existing design to bind a target protein of interest, e.g., a PD-L1 binding ISVD or a PD-1 binding ISVD or adding moieties that enhance efficacy like the addition of a half-life extender domain, e.g., an albumin binding ISVD.

### Methods

To obtain multivalent constructs capable of activating both IL-2R subunits IL-2Rβ and IL-2Rγ, an IL-2Rβ binding ISVD was linked to an IL-2Rγ binding ISVD using a Gly-Ser linker (e.g., SEQ ID NO: 58 also labeled “20GS”). The specific order of the respective ISVDs was varied within the different constructs.

For some constructs, the anti-ILR2β/anti-IL-2Rγ constructs were further linked with a bivalent anti-PD-L1 ISVD (see e.g., labeled “A0255010F11(E1D)-20GS (SEQ ID NO: 58)-A0255010F11” in Table 6 below) at the N-terminal position using the ISVD constructs binding to IL-2Rβ and IL-2Rγ linked via a 20GS (SEQ ID NO: 58) linker.

To increase the in vivo half-life, the multivalent ISVD constructs were additionally extended with a C-terminal albumin binding ISVD (ALB23002; SEQ ID NO: 90) using a 20GS (SEQ ID NO: 58) linker. Multivalent ISVD constructs designed without a C-terminal albumin binding ISVD included a terminal FLAG3His6 (SEQ ID NO: 470) Tag and were expressed in Pichia pastoris and purified according to standard protocols. Multivalent ISVD constructs designed with a C-terminal albumin binding ISVD were purified according to a standard protein A purification protocol.

### Results

The name and a description of the multivalent ISVD constructs generated are set forth in Table 7 and sequences are provided in SEQ ID NOs: 361-386.

### Conclusion

This Example demonstrates that multivalent anti-IL-2Rβγ ISVD constructs can be engineered to include additional modular moieties, for example, that specifically bind to a target protein of interest and/or that can enhance the therapeutic efficacy of the multispecific binding proteins of the disclosure (e.g., increasing serum half-life).

TABLE 6
Description of multivalent ISVD constructs (BB = ISVD building block; LL = linker)

| Sample ID | BB1 (target) | LL1 | BB2 (target) | LL2 | BB3 | LL3 | BB4 | LL-BB4 |
|---|---|---|---|---|---|---|---|---|
| A030900012 | A0255010F11 | 20GS | A0255010F11 | 20GS | A0291031G03 | 20GS | A0291142F04 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 58) | | |
| A030900015 | A0255010F11 | 20GS | A0255010F11 | 20GS | A0291142F04 | 20GS | A0291031G03 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 58) | | |
| A029100132 | / | / | / | / | A0291031G03 | 20GS | A0291159F01 | 20GS(SEQ ID NO: |
| | | | | | | (SEQ ID | | 58)-ALB23002-A |
| | | | | | | NO: 58) | | |
| A030900014 | A0255010F11 | 20GS | A0255010F11 | 20GS | A0291031G03 | 20GS | A0291159F01 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 58) | | |
| A029100133 | / | / | / | / | A0291159F01 | 20GS | A0291031G03 | 20GS (SEQ ID NO: |
| | | | | | | (SEQ ID | | 58)-ALB23002-A |
| | | | | | | NO: 58) | | |
| A030900004 | A0255010F11 | 20GS | A0255010F11 | 20GS | A0291159F01 | 20GS | A0291031G03 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 58) | | |
| A030900032 | A029400162 | 9GS | A029400162 | 20GS | A0291159F01 | 20GS | A0291031G03 | 20GS (SEQ ID NO: |
| | (PD1) | (SEQ ID | (PD1) | (SEQ ID | | (SEQ ID | | 58)-ALB23002-A |
| | | NO: 67) | | NO: 58) | | NO: 58) | | |
| A030900007 | A0255010F11 | 20GS | A0255010F11 | 20GS | A0291033D02 | 20GS | A0291142F04 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 58) | | |
| A029100151 | / | / | / | / | A0291142F04 | 20GS | A0291033D02 | 20GS (SEQ ID NO: |
| | | | | | | (SEQ ID | | 58)-ALB23002-A |
| | | | | | | NO: 58) | | |
| A030900009 | A0255010F11 | 20GS | A0255010F11 | 20GS | A0291142F04 | 20GS | A0291033D02 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 58) | | |
| A030900034 | A029400162 | 9GS | A029400162 | 20GS | A0291142F04 | 20GS | A0291033D02 | 20GS (SEQ ID NO: |
| | (PD1) | (SEQ ID | (PD1) | (SEQ ID | | (SEQ ID | | 58)-ALB23002-A |
| | | NO: 67) | | NO: 58) | | NO: 58) | | |
| A030900024 | A025501511 | 20GS | A025501511 | 20GS | A0291142F04 | 9GS | A0291033D02 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 67) | | |
| A030900026 | A025501511 | 20GS | A025501511 | 20GS | A0291142F04 | 5GS | A0291033D02 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 6) | | |
| A029100150 | / | / | / | / | A0291033D02 | 20GS | A0291159F01 | 20GS (SEQ ID NO: |
| | | | | | | (SEQ ID | | 58)-ALB23002-A |
| | | | | | | NO: 58) | | |
| A030900008 | A0255010F11 | 20GS | A0255010F11 | 20GS | A0291033D02 | 20GS | A0291159F01 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 58) | | |
| A030900035 | A029400162 | 9GS | A029400162 | 20GS | A0291033D02 | 20GS | A0291159F01 | 20GS (SEQ ID NO: |
| | (PD1) | (SEQ ID | (PD1) | (SEQ ID | | (SEQ ID | | 58)-ALB23002-A |
| | | NO: 67) | | NO: 58) | | NO: 58) | | |
| A030900021 | A025501511 | 20GS | A025501511 | 20GS | A0291033D02 | 9GS | A0291159F01 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 67) | | |
| A030900023 | A025501511 | 20GS | A025501511 | 20GS | A0291033D02 | 5GS | A0291159F01 | 20GS (SEQ ID NO: |
| | (PDL1) | (SEC | (PDL1) | (SEQ ID | | (SEQ ID | | 58)-ALB23002-A |
| | | NO: 58) | | NO: 58) | | NO: 6) | | |
| A029100152 | / | / | / | / | A0291159F01 | 20GS | A0291033D02 | 20GS (SEQ ID NO: |
| | | | | | | (SEQ ID | | 58)-ALB23002-A |
| | | | | | | NO: 58) | | |
| A030900011 | A0255010F11 | 20GS | A0255010F11 | 20GS | A0291159F01 | 20GS | A0291033D02 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 58) | | |
| A030900036 | A029400162 | 9GS | A029400162 | 20GS | A0291159F01 | 20GS | A0291033D02 | 20GS (SEQ ID NO: |
| | (PD1) | (SEQ ID | (PD1) | (SEQ ID | | (SEQ ID | | 58)-ALB23002-A |
| | | NO: 67) | | NO: 58) | | NO: 58) | | |
| A030900025 | A025501511 | 20GS | A025501511 | 20GS | A0291159F01 | 9GS | A0291033D02 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 67) | | |
| A030900027 | A025501511 | 20GS | A025501511 | 20GS | A0291159F01 | 5GS | A0291033D02 | 20GS (SEQ ID NO: |
| | (PDL1) | (SEQ ID | (PDL1) | (SEQ ID | | (SEQ ID | | 58)-ALB23002-A |
| | | NO: 58) | | NO: 58) | | NO: 6) | | |

TABLE 7
Sequences of individual anti-PD-1 VHH and multivalent anti-IL-2Rβγ
constructs (“ID” refers to the SEQ ID NO. attributed to each sequence)

| Name | ID | Sequence |
|---|---|---|
| A0294047E02 | 361 | EVQLVESGGGLVQSGGSLRLSCAATGFTLDYYVIGWFRQAPGKEREGVACL |
| (anti-PD-1 | | SSNGDRINYADSVKGRFTISRDIAKNTVYLQMNSLKPEDTAVYHCAAGTST |
| VHH) | | TVRDMCGIMYLYDYWAQGTLVTVSS |
| A0294047E02 (E1D, | 362 | DVQLVESGGGVVQPGGSLRLSCAATGFTLDYYVIGWFRQAPGKEREGVACL |
| L11V, S14P, K83R, | | SSNGDRINYADSVKGRFTISRDIAKNTVYLQMNSLRPEDTALYHCAAGTST |
| V89L) | | TVRDMCGIMYLYDYWAQGTLVTVSS |
| A0294047E02 (L11V, | 363 | EVQLVESGGGVVQPGGSLRLSCAATGFTLDYYVIGWFRQAPGKEREGVACL |
| S14P, K83R, V89L) | | SSNGDRINYADSVKGRFTISRDIAKNTVYLQMNSLRPEDTALYHCAAGTST |
| | | TVRDMCGIMYLYDYWAQGTLVTVSS |
| A030900012 | 364 | DVQLVESGGGLVQPGGSLRLSCAASGRTFSGNTMGWFRQAPGKEREFVAAI |
| | | SSSGRNTNYADPVEVRFTISRDNAKNTVYLQMNSLQPEDTAVYYCAASSWA |
| | | AAAGTIANIYDYWGQGTLVTVSSGGGGSGGGGSGGGGSGGGGSEVQLVESG |
| | | GGLVQPGGSLRLSCAASGRTFSGNTMGWFRQAPGKEREFVAAISSSGRNTN |
| | | YADPVEVRFTISRDNAKNTVYLQMNSLQPEDTAVYYCAASSWAAAAGTIAN |
| | | IYDYWGQGTLVTVSSGGGGSGGGGSGGGGSGGGGSEVQLVESGGGLVQAGG |
| | | SLRLSCVASGRTFSATNMGWFRQAPGKEREFVGIISESGRNTDYADSVKGR |
| | | FTITRDNAKSTVYLQMNNLKPEDTAVYYCALGRDYFATDTRQYNYWGLGTL |
| | | VTVSSGGGGSGGGGSGGGGSGGGGSEVQLVESGGGWVQPGGSLRLSCSPSE |
| | | RLSSIDIMGWYRQTPGKEREWVATITGDDTTHYENSVKGRFTISRDTAKNM |
| | | VYLQMNSLEPEDTAVYYCNAGILPREEYWGQGTLVTVSSGGGGSGGGGSGG |
| | | GGSGGGGSEVQLVESGGGVVQPGGSLRLSCAASGFTFRSFGMSWVRQAPGK |
| | | GPEWVSSISGSGSDTLYADSVKGRFTISRDNSKNTLYLQMNSLRPEDTALY |
| | | YCTIGGSLSRSSQGTLVTVSSA |
| A030900015 | 365 | DVQLVESGGGLVQPGGSLRLSCAASGRTFSGNTMGWFRQAPGKEREFVAAI |
| | | SSSGRNTNYADPVEVRFTISRDNAKNTVYLQMNSLQPEDTAVYYCAASSWA |
| | | AAAGTIANIYDYWGQGTLVTVSSGGGGSGGGGSGGGGSGGGGSEVQLVESG |
| | | GGLVQPGGSLRLSCAASGRTFSGNTMGWFRQAPGKEREFVAAISSSGRNTN |
| | | YADPVEVRFTISRDNAKNTVYLQMNSLQPEDTAVYYCAASSWAAAAGTIAN |
| | | IYDYWGQGTLVTVSSGGGGSGGGGSGGGGSGGGGSEVQLVESGGGWVQPGG |
| | | SLRLSCSPSERLSSIDIMGWYRQTPGKEREWVATITGDDTTHYENSVKGRF |
| | | TISRDTAKNMVYLQMNSLEPEDTAVYYCNAGILPREEYWGQGTLVTVSSGG |
| | | GGSGGGGSGGGGSGGGGSEVQLVESGGGLVQAGGSLRLSCVASGRTFSATN |
| | | MGWFRQAPGKEREFVGIISESGRNTDYADSVKGRFTITRDNAKSTVYLQMN |
| | | NLKPEDTAVYYCALGRDYFATDTRQYNYWGLGTLVTVSSGGGGSGGGGSGG |
| | | GGSGGGGSEVQLVESGGGVVQPGGSLRLSCAASGFTFRSFGMSWVRQAPGK |
| | | GPEWVSSISGSGSDTLYADSVKGRFTISRDNSKNTLYLQMNSLRPEDTALY |
| | | YCTIGGSLSRSSQGTLVTVSSA |
| A029100132 | 366 | DVQLVESGGGLVQAGGSLRLSCVASGRTFSATNMGWFRQAPGKEREFVGII |
| | | SESGRNTDYADSVKGRFTITRDNAKSTVYLQMNNLKPEDTAVYYCALGRDY |
| | | FATDTRQYNYWGLGTLVTVSSGGGGSGGGGSGGGGSGGGGSEVQLVESGGG |
| | | LVQAGDSTTISCVASGGSFATYAFGWFRQAPGKEREFVATISQRGSTSYYS |
| | | DSVEGRFTISKDNAKSTVYLQMNSLQPEDTAVYYCAARYYGVDYRSTSYDF |
| | | WGQGTLVTVSSGGGGSGGGGSGGGGSGGGGSEVQLVESGGGVVQPGGSLRL |
| | | SCAASGFTFRSFGMSWVRQAPGKGPEWVSSISGSGSDTLYADSVKGRFTIS |
| | | RDNSKNTLYLQMNSLRPEDTALYYCTIGGSLSRSSQGTLVTVSSA |
| A030900014 | 367 | DVQLVESGGGLVQPGGSLRLSCAASGRTFSGNTMGWFRQAPGKEREFVAAI |
| | | SSSGRNTNYADPVEVRFTISRDNAKNTVYLQMNSLQPEDTAVYYCAASSWA |
| | | AAAGTIANIYDYWGQGTLVTVSSGGGGGGGGSGGGGSGGGGSEVQLVESG |
| | | GGLVQPGGSLRLSCAASGRTFSGNTMGWFRQAPGKEREFVAAISSSGRNTN |
| | | YADPVEVRFTISRDNAKNTVYLQMNSLQPEDTAVYYCAASSWAAAAGTIAN |
| | | IYDYWGQGTLVTVSSGGGGSGGGGSGGGGSGGGGSEVQLVESGGGLVQAGG |
| | | SLRLSCVASGRTFSATNMGWFRQAPGKEREFVGIISESGRNTDYADSVKGR |
| | | FTITRDNAKSTVYLQMNNLKPEDTAVYYCALGRDYFATDTRQYNYWGLGTL |
| | | VTVSSGGGGSGGGGSGGGGSGGGGSEVQLVESGGGLVQAGDSTTISCVASG |
| | | GSFATYAFGWFRQAPGKEREFVATISQRGSTSYYSDSVEGRFTISKDNAKS |
| | | TVYLQMNSLQPEDTAVYYCAARYYGVDYRSTSYDFWGQGTLVTVSSGGGGS |
| | | GGGGSGGGGSGGGGSEVQLVESGGGVVQPGGSLRLSCAASGFTFRSFGMSW |
| | | VRQAPGKGPEWVSSISGSGSDTLYADSVKGRFTISRDNSKNTLYLQMNSLR |
| | | PEDTALYYCTIGGSLSRSSQGTLVTVSSA |
| A029100133 | 368 | DVQLVESGGGLVQAGDSTTISCVASGGSFATYAFGWFRQAPGKEREFVATI |
| | | SQRGSTSYYSDSVEGRFTISKDNAKSTVYLQMNSLQPEDTAVYYCAARYYG |
| | | VDYRSTSYDFWGQGTLVTVSSGGGGSGGGGSGGGGSGGGGSEVQLVESGGG |
| | | LVQAGGSLRLSCVASGRTFSATNMGWFRQAPGKEREFVGIISESGRNTDYA |
| | | DSVKGRFTITRDNAKSTVYLQMNNLKPEDTAVYYCALGRDYFATDTRQYNY |
| | | WGLGTLVTVSSGGGGSGGGGSGGGGSGGGGSEVQLVESGGGVVQPGGSLRL |
| | | SCAASGFTFRSFGMSWVRQAPGKGPEWVSSISGSGSDTLYADSVKGRFTIS |
| | | RDNSKNTLYLQMNSLRPEDTALYYCTIGGSLSRSSQGTLVTVSSA |
| A030900004 | 369 | DVQLVESGGGLVQPGGSLRLSCAASGRTFSGNTMGWFRQAPGKEREFVAAI |
| | | SSSGRNTNYADPVEVRFTISRDNAKNTVYLQMNSLQPEDTAVYYCAASSWA |
| | | AAAGTIANIYDYWGQGTLVTVSSGGGGSGGGGSGGGGSGGGGSEVQLVESG |
| | | GGLVQPGGSLRLSCAASGRTFSGNTMGWFRQAPGKEREFVAAISSSGRNTN |
| | | YADPVEVRFTISRDNAKNTVYLQMNSLQPEDTAVYYCAASSWAAAAGTIAN |
| | | IYDYWGQGTLVTVSSGGGGSGGGGSGGGGSGGGGSEVQLVESGGGLVQAGD |
| | | STTISCVASGGSFATYAFGWFRQAPGKEREFVATISQRGSTSYYSDSVEGR |
| | | FTISKDNAKSTVYLQMNSLQPEDTAVYYCAARYYGVDYRSTSYDFWGQGTL |
| | | VTVSSGGGGSGGGGSGGGGSGGGGSEVQLVESGGGLVQAGGSLRLSCVASG |
| | | RTFSATNMGWFRQAPGKEREFVGIISESGRNTDYADSVKGRFTITRDNAKS |
| | | TVYLQMNNLKPEDTAVYYCALGRDYFATDTRQYNYWGLGTLVTVSSGGGGS |
| | | GGGGSGGGGSGGGGSEVQLVESGGGVVQPGGSLRLSCAASGFTFRSFGMSW |
| | | VRQAPGKGPEWVSSISGSGSDTLYADSVKGRFTISRDNSKNTLYLQMNSLR |
| | | PEDTALYYCTIGGSLSRSSQGTLVTVSSA |
| A030900032 = | 370 | DVQLVESGGGVVQPGGSLRLSCAATGFTLDYYVIGWFRQAPGKEREGVACL |
| A0294047E02 (E1D, | | SSNGDRINYADSVKGRFTISRDIAKNTVYLQMNSLRPEDTALYHCAAGTST |
| L11V, S14P, K83R, | | TVRDMCGIMYLYDYWAQGTLVTVSSGGGGSGGGSEVQLVESGGGVVQPGGS |
| V89L)-9GS | | LRLSCAATGFTLDYYVIGWFRQAPGKEREGVACLSSNGDRINYADSVKGRF |
| (SEQ ID NO: | | TISRDIAKNTVYLQMNSLRPEDTALYHCAAGTSTTVRDMCGIMYLYDYWAQ |
| 67) - | | GTLVTVSSGGGGSGGGGSGGGGSGGGGSEVQLVESGGGLVQAGDSTTISCV |
| A0294047E02 (L11V, | | ASGGSFATYAFGWFRQAPGKEREFVATISQRGSTSYYSDSVEGRFTISKDN |
| S14P, K83R, | | AKSTVYLQMNSLQPEDTAVYYCAARYYGVDYRSTSYDFWGQGTLVTVSSGG |
| V89L)-20GS (SEQ | | GGSGGGGSGGGGSGGGGSEVQLVESGGGLVQAGGSLRLSCVASGRTFSATN |
| ID NO: 58) - | | MGWFRQAPGKEREFVGIISESGRNTDYADSVKGRFTITRDNAKSTVYLQMN |
| A0291159F01- | | NLKPEDTAVYYCALGRDYFATDTRQYNYWGLGTLVTVSSGGGGSGGGGSGG |
| 20GS (SEQ ID | | GGSGGGGSEVQLVESGGGVVQPGGSLRLSCAASGFTFRSFGMSWVRQAPGK |
| NO: 58) - | | GPEWVSSISGSGSDTLYADSVKGRFTISRDNSKNTLYLQMNSLRPEDTALY |
| A0291031G03- | | YCTIGGSLSRSSQGTLVTVSSA |
| 20GS (SEQ ID | | |
| NO: 58) - | | |
| ALB23002-A] | | |
| A030900007 | 371 | DVQLVESGGGLVQPGGSLRLSCAASGRTFSGNTMGWFRQAPGKEREFVAAI |
| | | SSSGRNTNYADPVEVRFTISRDNAKNTVYLQMNSLQPEDTAVYYCAASSWA |
| | | AAAGTIANIYDYWGQGTLVTVSSGGGGSGGGGSGGGGSGGGGSEVQLVESG |
| | | GGLVQPGGSLRLSCAASGRTFSGNTMGWFRQAPGKEREFVAAISSSGRNTN |
| | | YADPVEVRFTISRDNAKNTVYLQMNSLQPEDTAVYYCAASSWAAAAGTIAN |
| | | IYDYWGQGTLVTVSSGGGGSGGGGSGGGGSGGGGSEVQLVESGGGLVQAGG |
| | | SLRLSCAASGRSFSSYYMGWFRQAPGKEREFVAGINWSGRSTHYADSVKGR |
| | | FTISRDNAKNTVYLQMNSLKPEDRAAYYCAADSAPSTRIGVRSLWDDYDYW |
| | | GQGTLVTVSSGGGGSGGGGSGGGGSGGGGSEVQLVESGGGWVQPGGSLRLS |
| | | CSPSERLSSIDIMGWYRQTPGKEREWVATITGDDTTHYENSVKGRFTISRD |
| | | TAKNMVYLQMNSLEPEDTAVYYCNAGILPREEYWGQGTLVTVSSGGGGSGG |
| | | GGSGGGGSGGGGSEVQLVESGGGVVQPGGSLRLSCAASGFTFRSFGMSWVR |
| | | QAPGKGPEWVSSISGSGSDTLYADSVKGRFTISRDNSKNTLYLQMNSLRPE |
| | | DTALYYCTIGGSLSRSSQGTLVTVSSA |
| A029100151 | 372 | DVQLVESGGGWVQPGGSLRLSCSPSERLSSIDIMGWYRQTPGKEREWVATI |
| | | TGDDTTHYENSVKGRFTISRDTAKNMVYLQMNSLEPEDTAVYYCNAGILPR |
| | | EEYWGQGTLVTVSSGGGGSGGGGSGGGGSGGGGSEVQLVESGGGLVQAGGS |
| | | LRLSCAASGRSFSSYYMGWFRQAPGKEREFVAGINWSGRSTHYADSVKGRF |
| | | TISRDNAKNTVYLQMNSLKPEDRAAYYCAADSAPSTRIGVRSLWDDYDYWG |
| | | QGTLVTVSSGGGGSGGGGSGGGGSGGGGSEVQLVESGGGVVQPGGSLRLSC |
| | | AASGFTFRSFGMSWVRQAPGKGPEWVSSISGSGSDTLYADSVKGRFTISRD |
| | | NSKNTLYLQMNSLRPEDTALYYCTIGGSLSRSSQGTLVTVSSA |
| A030900009 | 373 | DVQLVESGGGLVQPGGSLRLSCAASGRTFSGNTMGWFRQAPGKEREFVAAI |
| | | SSSGRNTNYADPVEVRETISRDNAKNTVYLQMNSLQPEDTAVYYCAASSWA |
| | | AAAGTIANIYDYWGQGTLVTVSSGGGGSGGGGSGGGGSGGGGSEVQLVESG |
| | | GGLVQPGGSLRLSCAASGRTFSGNTMGWFRQAPGKEREFVAAISSSGRNTN |
| | | YADPVEVRFTISRDNAKNTVYLQMNSLQPEDTAVYYCAASSWAAAAGTIAN |
| | | IYDYWGQGTLVTVSSGGGGSGGGGSGGGGSGGGGSEVQLVESGGGWVQPGG |
| | | SLRLSCSPSERLSSIDIMGWYRQTPGKEREWVATITGDDTTHYENSVKGRF |
| | | TISRDTAKNMVYLQMNSLEPEDTAVYYCNAGILPREEYWGQGTLVTVSSGG |
| | | GGSGGGGSGGGGSGGGGSEVQLVESGGGLVQAGGSLRLSCAASGRSFSSYY |
| | | MGWFRQAPGKEREFVAGINWSGRSTHYADSVKGRFTISRDNAKNTVYLQMN |
| | | SLKPEDRAAYYCAADSAPSTRIGVRSLWDDYDYWGQGTLVTVSSGGGGSGG |
| | | GGSGGGGSGGGGSEVQLVESGGGVVQPGGSLRLSCAASGFTFRSFGMSWVR |
| | | QAPGKGPEWVSSISGSGSDTLYADSVKGRFTISRDNSKNTLYLQMNSLRPE |
| | | DTALYYCTIGGSLSRSSQGTLVTVSSA |
| A030900034 | 374 | DVQLVESGGGVVQPGGSLRLSCAATGFTLDYYVIGWFRQAPGKEREGVACL |
| A0294047E02 (E1D, | | SSNGDRINYADSVKGRFTISRDIAKNTVYLQMNSLRPEDTALYHCAAGTST |
| L11V, S14P, | | TVRDMCGIMYLYDYWAQGTLVTVSSGGGGSGGGSEVQLVESGGGVVQPGGS |
| K83R,V89L)-9GS | | LRLSCAATGFTLDYYVIGWFRQAPGKEREGVACLSSNGDRINYADSVKGRF |
| (SEQ ID NO: | | TISRDIAKNTVYLQMNSLRPEDTALYHCAAGTSTTVRDMCGIMYLYDYWAQ |
| 67) - | | GTLVTVSSGGGGSGGGGSGGGGSGGGGSEVQLVESGGGWVQPGGSLRLSCS |
| A0294047E02 (L11V, | | PSERLSSIDIMGWYRQTPGKEREWVATITGDDTTHYENSVKGRFTISRDTA |
| S14P, K83R, | | KNMVYLQMNSLEPEDTAVYYCNAGILPREEYWGQGTLVTVSSGGGGSGGGG |
| V89L)-20GS (SEQ | | SGGGGSGGGGSEVQLVESGGGLVQAGGSLRLSCAASGRSFSSYYMGWFRQA |
| ID NO: 58) - | | PGKEREFVAGINWSGRSTHYADSVKGRFTISRDNAKNTVYLQMNSLKPEDR |
| A0291142F04- | | AAYYCAADSAPSTRIGVRSLWDDYDYWGQGTLVTVSSGGGGSGGGGSGGGG |
| 20GS (SEQ ID | | SGGGGSEVQLVESGGGVVQPGGSLRLSCAASGFTFRSFGMSWVRQAPGKGP |
| NO: 58) - | | EWVSSISGSGSDTLYADSVKGRFTISRDNSKNTLYLQMNSLRPEDTALYYC |
| A0291033D02 (C67F)- | | TIGGSLSRSSQGTLVTVSSA |
| 20GS (SEQ | | |
| ID NO: 58) - | | |
| ALB23002-A] | | |
| A030900024 | 375 | DVQLVESGGGVVQPGGSLRLSCAASGRTFSGNTMGWFRQAPGKEREFVAAI |
| | | SSTGRNTNYADSVEGRFTISRDNSKNTVYLQMNSLRPEDTALYYCAASSWA |
| | | AAAGTIANIYDYWGQGTLVTVSSGGGGSGGGGSGGGGSGGGGSEVQLVESG |
| | | GGVVQPGGSLRLSCAASGRTFSGNTMGWFRQAPGKEREFVAAISSTGRNTN |
| | | YADSVEGRETISRDNSKNTVYLQMNSLRPEDTALYYCAASSWAAAAGTIAN |
| | | IYDYWGQGTLVTVSSGGGGSGGGGSGGGGSGGGGSEVQLVESGGGWVQPGG |
| | | SLRLSCSPSERLSSIDIMGWYRQTPGKEREWVATITGDDTTHYENSVKGRF |
| | | TISRDTAKNMVYLQMNSLEPEDTAVYYCNAGILPREEYWGQGTLVTVSSGG |
| | | GGSGGGSEVQLVESGGGLVQAGGSLRLSCAASGRSFSSYYMGWFRQAPGKE |
| | | REFVAGINWSGRSTHYADSVKGRFTISRDNAKNTVYLQMNSLKPEDRAAYY |
| | | CAADSAPSTRIGVRSLWDDYDYWGQGTLVTVSSGGGGSGGGGSGGGGSGGG |
| | | GSEVQLVESGGGVVQPGGSLRLSCAASGFTFRSFGMSWVRQAPGKGPEWVS |
| | | SISGSGSDTLYADSVKGRFTISRDNSKNTLYLQMNSLRPEDTALYYCTIGG |
| | | SLSRSSQGTLVTVSSA |
| A030900026 | 376 | DVQLVESGGGVVQPGGSLRLSCAASGRTFSGNTMGWFRQAPGKEREFVAAI |
| | | SSTGRNTNYADSVEGRETISRDNSKNTVYLQMNSLRPEDTALYYCAASSWA |
| | | AAAGTIANIYDYWGQGTLVTVSSGGGGSGGGGSGGGGSGGGGSEVQLVESG |
| | | GGVVQPGGSLRLSCAASGRTFSGNTMGWFRQAPGKEREFVAAISSTGRNTN |
| | | YADSVEGRFTISRDNSKNTVYLQMNSLRPEDTALYYCAASSWAAAAGTIAN |
| | | IYDYWGQGTLVTVSSGGGGSGGGGSGGGGSGGGGSEVQLVESGGGWVQPGG |
| | | SLRLSCSPSERLSSIDIMGWYRQTPGKEREWVATITGDDTTHYENSVKGRF |
| | | TISRDTAKNMVYLQMNSLEPEDTAVYYCNAGILPREEYWGQGTLVTVSSGG |
| | | GGSEVQLVESGGGLVQAGGSLRLSCAASGRSFSSYYMGWFRQAPGKEREFV |
| | | AGINWSGRSTHYADSVKGRFTISRDNAKNTVYLQMNSLKPEDRAAYYCAAD |
| | | SAPSTRIGVRSLWDDYDYWGQGTLVTVSSGGGGSGGGGSGGGGSGGGGSEV |
| | | QLVESGGGVVQPGGSLRLSCAASGFTFRSFGMSWVRQAPGKGPEWVSSISG |
| | | SGSDTLYADSVKGRFTISRDNSKNTLYLQMNSLRPEDTALYYCTIGGSLSR |
| | | SSQGTLVTVSSA |
| A029100150 | 377 | DVQLVESGGGLVQAGGSLRLSCAASGRSFSSYYMGWFRQAPGKEREFVAGI |
| | | NWSGRSTHYADSVKGRFTISRDNAKNTVYLQMNSLKPEDRAAYYCAADSAP |
| | | STRIGVRSLWDDYDYWGQGTLVTVSSGGGGSGGGGSGGGGSGGGGSEVQLV |
| | | ESGGGLVQAGDSTTISCVASGGSFATYAFGWFRQAPGKEREFVATISQRGS |
| | | TSYYSDSVEGRFTISKDNAKSTVYLQMNSLQPEDTAVYYCAARYYGVDYRS |
| | | TSYDFWGQGTLVTVSSGGGGSGGGGSGGGGSGGGGSEVQLVESGGGVVQPG |
| | | GSLRLSCAASGFTFRSFGMSWVRQAPGKGPEWVSSISGSGSDTLYADSVKG |
| | | RFTISRDNSKNTLYLQMNSLRPEDTALYYCTIGGSLSRSSQGTLVTVSSA |
| A030900008 | 378 | DVQLVESGGGLVQPGGSLRLSCAASGRTFSGNTMGWFRQAPGKEREFVAAI |
| | | SSSGRNTNYADPVEVRFTISRDNAKNTVYLQMNSLQPEDTAVYYCAASSWA |
| | | AAAGTIANIYDYWGQGTLVTVSSGGGGSGGGGSGGGGSGGGGSEVQLVESG |
| | | GGLVQPGGSLRLSCAASGRTFSGNTMGWFRQAPGKEREFVAAISSSGRNTN |
| | | YADPVEVRFTISRDNAKNTVYLQMNSLQPEDTAVYYCAASSWAAAAGTIAN |
| | | IYDYWGQGTLVTVSSGGGGSGGGGSGGGGSGGGGSEVQLVESGGGLVQAGG |
| | | SLRLSCAASGRSFSSYYMGWFRQAPGKEREFVAGINWSGRSTHYADSVKGR |
| | | FTISRDNAKNTVYLQMNSLKPEDRAAYYCAADSAPSTRIGVRSLWDDYDYW |
| | | GQGTLVTVSSGGGGSGGGGSGGGGSGGGGSEVQLVESGGGLVQAGDSTTIS |
| | | CVASGGSFATYAFGWFRQAPGKEREFVATISQRGSTSYYSDSVEGRFTISK |
| | | DNAKSTVYLQMNSLQPEDTAVYYCAARYYGVDYRSTSYDFWGQGTLVTVSS |
| | | GGGGSGGGGSGGGGSGGGGSEVQLVESGGGVVQPGGSLRLSCAASGFTERS |
| | | FGMSWVRQAPGKGPEWVSSISGSGSDTLYADSVKGRFTISRDNSKNTLYLQ |
| | | MNSLRPEDTALYYCTIGGSLSRSSQGTLVTVSSA |
| A030900035 | 379 | DVQLVESGGGVVQPGGSLRLSCAATGFTLDYYVIGWFRQAPGKEREGVACL |
| | | SSNGDRINYADSVKGRFTISRDIAKNTVYLQMNSLRPEDTALYHCAAGTST |
| | | TVRDMCGIMYLYDYWAQGTLVTVSSGGGGSGGGSEVQLVESGGGVVQPGGS |
| | | LRLSCAATGFTLDYYVIGWFRQAPGKEREGVACLSSNGDRINYADSVKGRF |
| | | TISRDIAKNTVYLQMNSLRPEDTALYHCAAGTSTTVRDMCGIMYLYDYWAQ |
| | | GTLVTVSSGGGGSGGGGSGGGGSGGGGSEVQLVESGGGLVQAGGSLRLSCA |
| | | ASGRSFSSYYMGWFRQAPGKEREFVAGINWSGRSTHYADSVKGRFTISRDN |
| | | AKNTVYLQMNSLKPEDRAAYYCAADSAPSTRIGVRSLWDDYDYWGQGTLVT |
| | | VSSGGGGSGGGGSGGGGSGGGGSEVQLVESGGGLVQAGDSTTISCVASGGS |
| | | FATYAFGWFRQAPGKEREFVATISQRGSTSYYSDSVEGRFTISKDNAKSTV |
| | | YLQMNSLQPEDTAVYYCAARYYGVDYRSTSYDFWGQGTLVTVSSGGGGSGG |
| | | GGSGGGGSGGGGSEVQLVESGGGVVQPGGSLRLSCAASGFTFRSFGMSWVR |
| | | QAPGKGPEWVSSISGSGSDTLYADSVKGRFTISRDNSKNTLYLQMNSLRPE |
| | | DTALYYCTIGGSLSRSSQGTLVTVSSA |
| A030900021 | 380 | DVQLVESGGGVVQPGGSLRLSCAASGRTFSGNTMGWFRQAPGKEREFVAAI |
| | | SSTGRNTNYADSVEGRFTISRDNSKNTVYLQMNSLRPEDTALYYCAASSWA |
| | | AAAGTIANIYDYWGQGTLVTVSSGGGGSGGGGSGGGGSGGGGSEVQLVESG |
| | | GGVVQPGGSLRLSCAASGRTFSGNTMGWFRQAPGKEREFVAAISSTGRNTN |
| | | YADSVEGRFTISRDNSKNTVYLQMNSLRPEDTALYYCAASSWAAAAGTIAN |
| | | IYDYWGQGTLVTVSSGGGGSGGGGSGGGGSGGGGSEVQLVESGGGLVQAGG |
| | | SLRLSCAASGRSFSSYYMGWFRQAPGKEREFVAGINWSGRSTHYADSVKGR |
| | | FTISRDNAKNTVYLQMNSLKPEDRAAYYCAADSAPSTRIGVRSLWDDYDYW |
| | | GQGTLVTVSSGGGGSGGGSEVQLVESGGGLVQAGDSTTISCVASGGSFATY |
| | | AFGWFRQAPGKEREFVATISQRGSTSYYSDSVEGRFTISKDNAKSTVYLQM |
| | | NSLQPEDTAVYYCAARYYGVDYRSTSYDFWGQGTLVTVSSGGGGSGGGGSG |
| | | GGGSGGGGSEVQLVESGGGVVQPGGSLRLSCAASGFTFRSFGMSWVRQAPG |
| | | KGPEWVSSISGSGSDTLYADSVKGRFTISRDNSKNTLYLQMNSLRPEDTAL |
| | | YYCTIGGSLSRSSQGTLVTVSSA |
| A030900023 | 381 | DVQLVESGGGVVQPGGSLRLSCAASGRTFSGNTMGWFRQAPGKEREFVAAI |
| | | SSTGRNTNYADSVEGRFTISRDNSKNTVYLQMNSLRPEDTALYYCAASSWA |
| | | AAAGTIANIYDYWGQGTLVTVSSGGGGSGGGGSGGGGSGGGGSEVQLVESG |
| | | GGVVQPGGSLRLSCAASGRTFSGNTMGWFRQAPGKEREFVAAISSTGRNTN |
| | | YADSVEGRFTISRDNSKNTVYLQMNSLRPEDTALYYCAASSWAAAAGTIAN |
| | | IYDYWGQGTLVTVSSGGGGSGGGGSGGGGSGGGGSEVQLVESGGGLVQAGG |
| | | SLRLSCAASGRSFSSYYMGWFRQAPGKEREFVAGINWSGRSTHYADSVKGR |
| | | FTISRDNAKNTVYLQMNSLKPEDRAAYYCAADSAPSTRIGVRSLWDDYDYW |
| | | GQGTLVTVSSGGGGSEVQLVESGGGLVQAGDSTTISCVASGGSFATYAFGW |
| | | FRQAPGKEREFVATISQRGSTSYYSDSVEGRFTISKDNAKSTVYLQMNSLQ |
| | | PEDTAVYYCAARYYGVDYRSTSYDFWGQGTLVTVSSGGGGSGGGGSGGGGS |
| | | GGGGSEVQLVESGGGVVQPGGSLRLSCAASGFTFRSFGMSWVRQAPGKGPE |
| | | WVSSISGSGSDTLYADSVKGRFTISRDNSKNTLYLQMNSLRPEDTALYYCT |
| | | IGGSLSRSSQGTLVTVSSA |
| A029100152 | 382 | DVQLVESGGGLVQAGDSTTISCVASGGSFATYAFGWFRQAPGKEREFVATI |
| | | SQRGSTSYYSDSVEGRFTISKDNAKSTVYLQMNSLQPEDTAVYYCAARYYG |
| | | VDYRSTSYDFWGQGTLVTVSSGGGGSGGGGSGGGGSGGGGSEVQLVESGGG |
| | | LVQAGGSLRLSCAASGRSFSSYYMGWFRQAPGKEREFVAGINWSGRSTHYA |
| | | DSVKGRFTISRDNAKNTVYLQMNSLKPEDRAAYYCAADSAPSTRIGVRSLW |
| | | DDYDYWGQGTLVTVSSGGGGSGGGGSGGGGSGGGGSEVQLVESGGGVVQPG |
| | | GSLRLSCAASGFTFRSFGMSWVRQAPGKGPEWVSSISGSGSDTLYADSVKG |
| | | RFTISRDNSKNTLYLQMNSLRPEDTALYYCTIGGSLSRSSQGTLVTVSSA |
| A030900011 | 383 | DVQLVESGGGLVQPGGSLRLSCAASGRTFSGNTMGWFRQAPGKEREFVAAI |
| | | SSSGRNTNYADPVEVRFTISRDNAKNTVYLQMNSLQPEDTAVYYCAASSWA |
| | | AAAGTIANIYDYWGQGTLVTVSSGGGGSGGGGSGGGGSGGGGSEVQLVESG |
| | | GGLVQPGGSLRLSCAASGRTFSGNTMGWFRQAPGKEREFVAAISSSGRNTN |
| | | YADPVEVRFTISRDNAKNTVYLQMNSLQPEDTAVYYCAASSWAAAAGTIAN |
| | | IYDYWGQGTLVTVSSGGGGSGGGGSGGGGSGGGGSEVQLVESGGGLVQAGD |
| | | STTISCVASGGSFATYAFGWFRQAPGKEREFVATISQRGSTSYYSDSVEGR |
| | | FTISKDNAKSTVYLQMNSLQPEDTAVYYCAARYYGVDYRSTSYDFWGQGTL |
| | | VTVSSGGGGSGGGGSGGGGSGGGGSEVQLVESGGGLVQAGGSLRLSCAASG |
| | | RSFSSYYMGWFRQAPGKEREFVAGINWSGRSTHYADSVKGRFTISRDNAKN |
| | | TVYLQMNSLKPEDRAAYYCAADSAPSTRIGVRSLWDDYDYWGQGTLVTVSS |
| | | GGGGSGGGGSGGGGSGGGGSEVQLVESGGGVVQPGGSLRLSCAASGFTFRS |
| | | FGMSWVRQAPGKGPEWVSSISGSGSDTLYADSVKGRFTISRDNSKNTLYLQ |
| | | MNSLRPEDTALYYCTIGGSLSRSSQGTLVTVSSA |
| A030900036 | 384 | DVQLVESGGGVVQPGGSLRLSCAATGFTLDYYVIGWFRQAPGKEREGVACL |
| | | SSNGDRINYADSVKGRFTISRDIAKNTVYLQMNSLRPEDTALYHCAAGTST |
| | | TVRDMCGIMYLYDYWAQGTLVTVSSGGGGSGGGSEVQLVESGGGVVQPGGS |
| | | LRLSCAATGFTLDYYVIGWFRQAPGKEREGVACLSSNGDRINYADSVKGRF |
| | | TISRDIAKNTVYLQMNSLRPEDTALYHCAAGTSTTVRDMCGIMYLYDYWAQ |
| | | GTLVTVSSGGGGSGGGGSGGGGSGGGGSEVQLVESGGGLVQAGDSTTISCV |
| | | ASGGSFATYAFGWFRQAPGKEREFVATISQRGSTSYYSDSVEGRFTISKDN |
| | | AKSTVYLQMNSLQPEDTAVYYCAARYYGVDYRSTSYDFWGQGTLVTVSSGG |
| | | GGSGGGGSGGGGSGGGGSEVQLVESGGGLVQAGGSLRLSCAASGRSFSSYY |
| | | MGWFRQAPGKEREFVAGINWSGRSTHYADSVKGRFTISRDNAKNTVYLQMN |
| | | SLKPEDRAAYYCAADSAPSTRIGVRSLWDDYDYWGQGTLVTVSSGGGGSGG |
| | | GGSGGGGSGGGGSEVQLVESGGGVVQPGGSLRLSCAASGFTFRSFGMSWVR |
| | | QAPGKGPEWVSSISGSGSDTLYADSVKGRFTISRDNSKNTLYLQMNSLRPE |
| | | DTALYYCTIGGSLSRSSQGTLVTVSSA |
| A030900025 | 385 | DVQLVESGGGVVQPGGSLRLSCAASGRTFSGNTMGWFRQAPGKEREFVAAI |
| | | SSTGRNTNYADSVEGRFTISRDNSKNTVYLQMNSLRPEDTALYYCAASSWA |
| | | AAAGTIANIYDYWGQGTLVTVSSGGGGSGGGGSGGGGSGGGGSEVQLVESG |
| | | GGVVQPGGSLRLSCAASGRTFSGNTMGWFRQAPGKEREFVAAISSTGRNTN |
| | | YADSVEGRFTISRDNSKNTVYLQMNSLRPEDTALYYCAASSWAAAAGTIAN |
| | | IYDYWGQGTLVTVSSGGGGSGGGGSGGGGSGGGGSEVQLVESGGGLVQAGD |
| | | STTISCVASGGSFATYAFGWFRQAPGKEREFVATISQRGSTSYYSDSVEGR |
| | | FTISKDNAKSTVYLQMNSLQPEDTAVYYCAARYYGVDYRSTSYDFWGQGTL |
| | | VTVSSGGGGSGGGSEVQLVESGGGLVQAGGSLRLSCAASGRSFSSYYMGWF |
| | | RQAPGKEREFVAGINWSGRSTHYADSVKGRFTISRDNAKNTVYLQMNSLKP |
| | | EDRAAYYCAADSAPSTRIGVRSLWDDYDYWGQGTLVTVSSGGGGSGGGGSG |
| | | GGGSGGGGSEVQLVESGGGVVQPGGSLRLSCAASGFTFRSFGMSWVRQAPG |
| | | KGPEWVSSISGSGSDTLYADSVKGRFTISRDNSKNTLYLQMNSLRPEDTAL |
| | | YYCTIGGSLSRSSQGTLVTVSSA |
| A030900027 | 386 | DVQLVESGGGVVQPGGSLRLSCAASGRTFSGNTMGWFRQAPGKEREFVAAI |
| | | SSTGRNTNYADSVEGRFTISRDNSKNTVYLQMNSLRPEDTALYYCAASSWA |
| | | AAAGTIANIYDYWGQGTLVTVSSGGGGSGGGGSGGGGSGGGGSEVQLVESG |
| | | GGVVQPGGSLRLSCAASGRTFSGNTMGWFRQAPGKEREFVAAISSTGRNTN |
| | | YADSVEGRFTISRDNSKNTVYLQMNSLRPEDTALYYCAASSWAAAAGTIAN |
| | | IYDYWGQGTLVTVSSGGGGSGGGGSGGGGSGGGGSEVQLVESGGGLVQAGD |
| | | STTISCVASGGSFATYAFGWFRQAPGKEREFVATISQRGSTSYYSDSVEGR |
| | | FTISKDNAKSTVYLQMNSLQPEDTAVYYCAARYYGVDYRSTSYDFWGQGTL |
| | | VTVSSGGGGSEVQLVESGGGLVQAGGSLRLSCAASGRSFSSYYMGWFRQAP |
| | | GKEREFVAGINWSGRSTHYADSVKGRFTISRDNAKNTVYLQMNSLKPEDRA |
| | | AYYCAADSAPSTRIGVRSLWDDYDYWGQGTLVTVSSGGGGSGGGGSGGGGS |
| | | GGGGSEVQLVESGGGVVQPGGSLRLSCAASGFTFRSFGMSWVRQAPGKGPE |
| | | WVSSISGSGSDTLYADSVKGRFTISRDNSKNTLYLQMNSLRPEDTALYYCT |
| | | IGGSLSRSSQGTLVTVSSA |

### Example 3: Activity of the Multivalent Constructs in an IL2Rβγ STAT5 Reporter Assay

### Introduction

This Example tests the activity of the multivalent anti-IL-2Rβγ ISVD constructs when compared to a reference IL-2 variant via the IL-2/JAK/STAT5 signal transduction pathway. The IL-2 variant is set forth in SEQ ID NO: 419 and has the following amino acid sequence:

MYRMQLLSCIALSLALVTNSAPASSSTKKTQLQLEHLLLDLQMILNGINN
YKNPKLTRMLTAKFAMPKKATELKHLQCLEEELKPLEEVLNGAQSKNFHL
RPRDLISNINVIVLELKGSETTFMCEYADETATIVEFLNRWITFAQSIIS
TLTHHHHHH.

### Method

The parental STAT5 Leeporter™ Luciferase Reporter-Ba/F3 Cell Line (Abeomics, 14-135AGL) was transfected with either human or cynomolgus IL2Rγ-F2A-β (see Table 8). The reporter cell line contains a firefly luciferase gene driven by the STAT5 response element located upstream of the minimal TATA promoter. Upon activation of the IL2 receptor complex, endogenous STAT5 binds to the DNA response elements, inducing transcription of the luciferase reporter gene.

TABLE 8
STAT5 Leeporter ™ Luciferase Reporter-Ba/F3 Cell Line
transfected with human IL-2R, cyno IL-2R or human IL-21R

| Cell line name | Source sequence |
|---|---|
| Ba/F3 STAT5 Leeporter Luciferase | huIL2Rb: UniProtKB, P14784 |
| reporter human IL2Rg-F2A-b clone | huIL2Rg: UniProtKB, P31785 |
| 2D8 | |
| Ba/F3 STAT5 Leeporter Luciferase | cyIL2Rb: UniProtKB, Q38J85 |
| reporter cyno IL2Rg-F2A-b clone | (IL2RB_MACFA, similar as |
| 7A4 | rhesus IL2RbNP_001244989.1 |
| | cyIL2Rg UniProtKB - G7Q2Z6 |
| | (G7Q2Z6_MACFA). |
| Ba/F3 STAT5 Leeporter Luciferase | huIL21Rb UniProtKB, Q9HBE5 |
| reporter human IL21Rg_human | huIL2Rg: UniProtKB, P31785 |
| IL21Rb clone 9E7_clone 9E7 | |

Ba/F3 human or cynomolgus IL-2Rβγ transfected pSTAT5 reporter cells (5×10⁵ cells/mL) were incubated in T75 flasks in assay medium (RPMI 1640, GlutaMAX Supplement (Life technologies—Gibco, 61870)) supplemented with 10% heat inactivated FBS (Sigma, F7524) and incubated overnight in a 5% CO₂ atmosphere at 37° C. for starvation. After the overnight starvation, cells were harvested, washed with assay medium and transferred to white 96-well flat-bottom plates (Costar, 3917) in assay medium (4×10⁵ cells/ml). For analysis of concentration dependent STAT5 phosphorylation, serial dilutions of multivalent ISVD constructs in assay medium were added to the cells and incubated for 16 h in a 5% CO₂ atmosphere at 37° C. After 16 h of incubation, Leeporter™ Luciferase Assay reagent was diluted according to the instructions of the supplier. Briefly, 500 μl Substrate Reconstitution Solution was added to the vial with lyophilized substrate. The substrate was then diluted 1/100 in assay buffer (=complete assay solution). After incubation, the plates were equilibrated to ambient temperature for 10 to 15 minutes. 50 μl complete Assay solution was added per well. Luminescence was read using the Envision within 1-5 minutes after addition of complete assay solution to all wells.

### Results

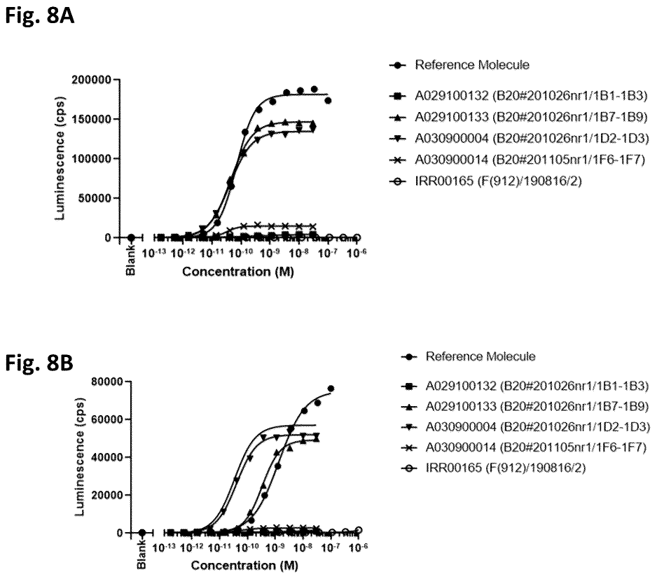

The STAT5 activity for some of the multivalent anti-IL-2Rβγ ISVD constructs are graphically displayed by luminescence read out in either Ba/F3 human (FIG. 8A) or cynomolgus (FIG. 8B) IL-2Rβγ transfected pSTAT5 reporter cells. Table 9 below displays the corresponding Ere (M) values and percent inhibition normalized to the IL-2v reference molecule. The IL-2v reference molecule is set forth in: SEQ ID NO: 420 and has the following amino acid sequence:

MYRMQLLSCIALSLALVTNSAPASSSTKKTQLQLEHLLLDLQMILNGINN
YKNPKLTRMLTAKFAMPKKATELKHLQCLEEELKPLEEVLNGAQSKNFHL
RPRDLISNINVIVLELKGSETTFMCEYADETATIVEFLNRWITFAQSIIS
TLT.

The majority of multivalent anti-IL-2Rβγ constructs exhibited agonist activity in the STAT5 reporter assay. Although the efficacy in the Ba/F3 cynomolgus IL2Rβγ STAT5 reporter assay was lower compared to the Ba/F3 human IL2Rβγ STAT5 reporter assay, a similar ranking of the multivalent constructs was seen and the difference in potency towards human or cynomolgus IL-2R was less than 10-fold.

TABLE 9
Global EC50 (M) and % inhibition (normalized to the IL-2 reference molecule)
of multivalent constructs in the Ba/F3 IL2Rβγ STAT5 reporter assay.

| | Ba/F3 human IL2Rβγ STAT5 reporter | | | | Ba/F3 cynomolgus IL2Rβγ STAT5 reporter | | | |
|---|---|---|---|---|---|---|---|---|
| | | LCI | UCI | | | LCI | UCI | |
| | EC50 | EC50 | EC50 | % of | EC50 | EC50 | EC50 | % of |
| Name | (M) | (M) | (M) | reference | (M) | (M) | (M) | reference |
| Human IL2v | 1.8E−10 | 1.5E−10 | 2.2E−10 | 100 | 1.4E−09 | 1.1E−09 | 2.0E−09 | 100 |
| A029100132 | 1.1E−09 | 8.0E−10 | 1.8E−09 | 5 | 9.7E−10 | 4.6E−10 | 5.2E−09 | 1 |
| A029100133 | 4.1E−11 | 3.6E−11 | 4.6E−11 | 88 | 3.4E−10 | 2.9E−10 | 3.9E−10 | 65 |
| A029100150 | 5.6E−11 | 4.8E−11 | 6.6E−11 | 71 | | | | |
| A029100151 | 6.5E−11 | 5.9E−11 | 7.1E−11 | 73 | | | | |
| A029100152 | 8.5E−11 | 8.1E−11 | 8.9E−11 | 61 | | | | |
| A030900004 | 3.8E−11 | 3.2E−11 | 4.6E−11 | 94 | 3.2E−11 | 2.7E−11 | 3.6E−11 | 79 |
| A030900007 | 1.3E−11 | 6.2E−12 | 2.0E−11 | 0 | | | | |
| A030900008 | 4.4E−11 | 3.4E−11 | 5.7E−11 | 45 | 2.8E−11 | 2.1E−11 | 3.8E−11 | 64 |
| A030900009 | 4.0E−11 | 3.4E−11 | 4.8E−11 | 71 | 2.6E−11 | 1.9E−11 | 3.4E−11 | 73 |
| A030900011 | 8.0E−11 | 6.5E−11 | 9.9E−11 | 63 | 4.3E−11 | 3.7E−11 | 5.1E−11 | 48 |
| A030900014 | 4.3E−11 | 3.5E−11 | 5.2E−11 | 10 | 6.0E−11 | 4.2E−11 | 8.9E−11 | 3 |
| A030900015 | 3.6E−11 | 3.2E−11 | 4.0E−11 | 72 | 3.4E−11 | 2.7E−11 | 4.3E−11 | 76 |
| A030900021 | 2.7E−10 | 2.4E−10 | 3.1E−10 | 40 | 1.2E−10 | 9.3E−11 | 1.4E−10 | 74 |
| A030900023 | 6.8E−11 | 4.2E−11 | 1.1E−10 | 33 | 2.6E−11 | 2.0E−11 | 3.5E−11 | 66 |
| A030900024 | 8.5E−11 | 4.4E−11 | 1.7E−10 | 74 | 2.8E−11 | 2.0E−11 | 3.7E−11 | 88 |
| A030900025 | 3.1E−10 | 2.4E−10 | 3.9E−10 | 22 | 1.0E−10 | 7.1E−11 | 1.5E−10 | 23 |
| A030900026 | 5.3E−11 | 4.2E−11 | 6.5E−11 | 67 | 2.8E−11 | 2.2E−11 | 3.3E−11 | 77 |
| A030900027 | 4.7E−10 | 3.5E−10 | 6.1E−10 | 8 | 1.8E−10 | 1.1E−10 | 2.7E−10 | 9 |
| A030900032 | 7.9E−11 | 6.5E−11 | 9.8E−11 | 86 | | | | |
| A030900034 | 5.4E−11 | 4.6E−11 | 6.3E−11 | 77 | | | | |
| A030900035 | 1.5E−10 | 1.3E−10 | 1.7E−10 | 50 | | | | |
| A030900036 | 1.1E−10 | 1.0E−10 | 1.3E−10 | 69 | | | | |
| Human IL-2 | 2.5E−10 | 2.0E−10 | 3.0E−10 | 94 | 2.7E−10 | 2.0E−10 | 3.9E−10 | 100 |

### Example 4: Activity of the Multivalent Constructs in IL21R STAT5 Reporter Assay

### Introduction

IL-2Rβ and IL-2γ subunits can also combine with and in different combinations and orders to form functional receptors for other cytokines as shown in FIG. 3. For example, the IL-2Rγ subunit can combine with either IL-4Rα, IL-7Rα, IL-9Rα, or IL-21R to form the receptors for IL-4, IL-7, IL-9, and IL-21, respectively.

This Example tests the specificity of the multivalent anti-IL-2Rβγ ISVD constructs to bind and activate a different receptor in the IL-2Rγ subunit family, e.g., IL-21R.

### Method

The parental STAT5 Leeporter™ Luciferase Reporter-Ba/F3 Cell Line (Abeomics, 14-135ACL) was transfected with human IL-21R (see Table 10). The reporter cell line contains a firefly luciferase gene driven by the STAT5 response element located upstream of the minimal TATA promoter. After activation of the IL-21R, endogenous STAT5 binds to the DNA response elements, inducing transcription of the luciferase reporter gene. The assay was performed as described in Example 3.

### Results

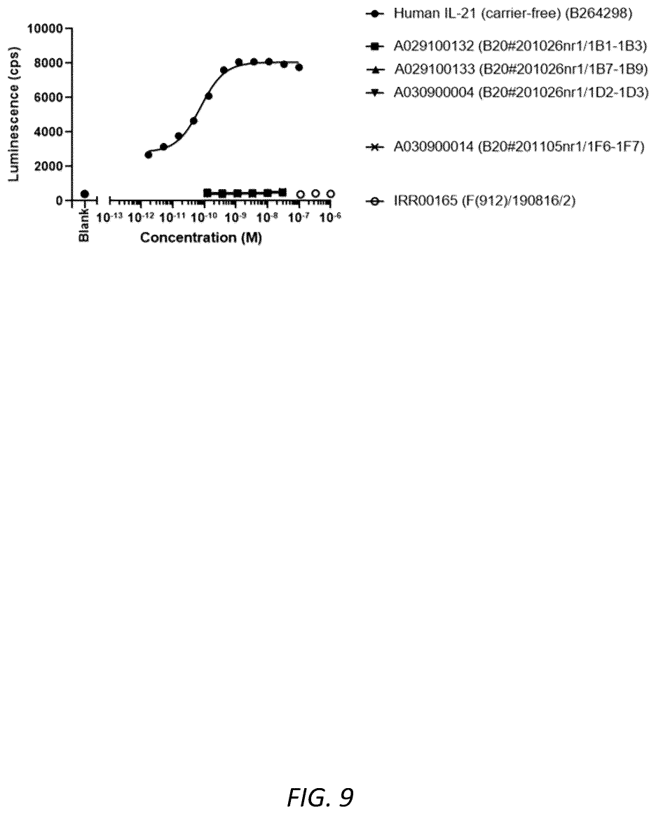

The IL-21R reporter activity for the different multivalent anti-IL-2Rβγ ISVD constructs is shown in FIG. 9 and summarized Table 10 below. The IL-21 reference molecule showed a dose dependent STAT5 activation while none of the multivalent ISVD constructs that were tested in the Ba/F3 human IL21 Rβγ STAT5 reporter assay were able to signal through the IL-21 receptor.

TABLE 10
Activity of multivalent constructs in the Ba/F3
human IL-21Rβγ STAT5 reporter assay.

| Name | Description |
|---|---|
| A029100132 | No functionality |
| A029100133 | No functionality |
| A030900004 | No functionality |
| A030900007 | No functionality |
| A030900008 | No functionality |
| A030900009 | No functionality |
| A030900011 | No functionality |
| A030900012 | No functionality |
| A030900014 | No functionality |
| A030900015 | No functionality |
| A030900021 | No functionality |
| A030900023 | No functionality |
| A030900024 | No functionality |
| A030900025 | No functionality |
| A030900026 | No functionality |
| A030900027 | No functionality |
| Human IL2v | No functionality |

### Conclusion

The multivalent anti-IL-2Rβγ ISVDs described herein are specific to receptors that contain both the IL-2Rβ and the IL-2Rγ subunits.

### Example 5: Multispecific Binding Proteins Bind to IL-2Rβγ Positive Cells and Degrade Target Protein

### Introduction

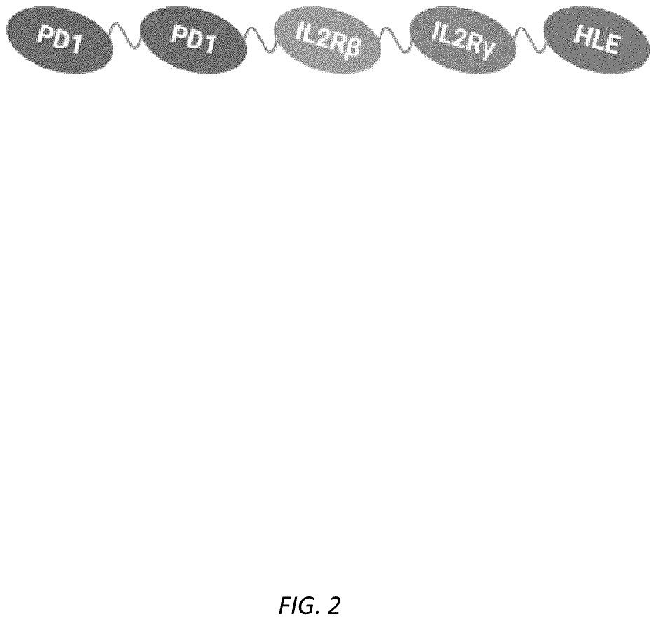

In this Example two different PD-1/IL-2Rβγ ISVD constructs as graphically displayed in FIG. 2 (amino acid sequences provided in Table 11) were analyzed for the ability to specifically bind to IL-2Rβγ at the cell surface of an IL-2Rβγ positive cell. A control molecule was used comprising PD-1 binding VHH but no VHH IL2Rβ or IL2Rγ binding domains). Experiments were performed using a Human Embryonic Kidney Cells (HEK) cell line that was transfected with the human IL-2 complex (IL-2 alpha (α), beta (β), and gamma (γ) chains) (HEK-IL2).

TABLE 11
Sequences of various multispecific ISVD constructs studied in Example 5

| SEQ ID | | |
|---|---|---|
| NO. | Construct | Amino Acid Sequence |
| 421 | A029403748 | DVQLVESGGGVVQPGGSLRLSCAATGFTLDYYVIGWFRQAPGKEREGVACLS |
| | (PD-1 binding | SNGDRINYADSVKGRFTISRDIAKNTVYLQMNSLRPEDTALYHCAAGTSTTV |
| | VHH molecule | RDMCGIMYLYDYWAQGTLVTVSSGGGGSGGGSEVQLVESGGGVVQPGGSLRL |
| | with a half- | SCAATGFTLDYYVIGWFRQAPGKEREGVACLSSNGDRINYADSVKGRFTISR |
| | life extension | DIAKNTVYLQMNSLRPEDTALYHCAAGTSTTVRDMCGIMYLYDYWAQGTLVT |
| | domain that | VSSGGGGSGGGGSGGGGSGGGGSEVQLVESGGGVVQPGGSLRLSCAASGFTF |
| | binds albumin) | RSFGMSWVRQAPGKGPEWVSSISGSGSDTLYADSVKGRFTISRDNSKNTLYL |
| | (PD-1-PD-1-HLE) | QMNSLRPEDTALYYCTIGGSLSRSSQGTLVTVSSA |
| 422 | A030900032 | DVQLVESGGGVVQPGGSLRLSCAATGFTLDYYVIGWFRQAPGKEREGVACLS |
| | (ISVD1 | SNGDRINYADSVKGRFTISRDIAKNTVYLQMNSLRPEDTALYHCAAGTSTTV |
| | construct 1- | RDMCGIMYLYDYWAQGTLVTVSSGGGGSGGGSEVQLVESGGGVVQPGGSLRL |
| | construct | SCAATGFTLDYYVIGWFRQAPGKEREGVACLSSNGDRINYADSVKGRFTISR |
| | comprising PD-1 | DIAKNTVYLQMNSLRPEDTALYHCAAGTSTTVRDMCGIMYLYDYWAQGTLVT |
| | binding domains | VSSGGGGSGGGGSGGGGSGGGGSEVQLVESGGGLVQAGDSTTISCVASGGSF |
| | and IL2Rβ and | ATYAFGWFRQAPGKEREFVATISQRGSTSYYSDSVEGRFTISKDNAKSTVYL |
| | IL2Rγ binding | QMNSLQPEDTAVYYCAARYYGVDYRSTSYDFWGQGTLVTVSSGGGGSGGGGS |
| | domains, and a | GGGGSGGGGSEVQLVESGGGLVQAGGSLRLSCVASGRTFSATNMGWFRQAPG |
| | half-life | KEREFVGIISESGRNTDYADSVKGRFTITRDNAKSTVYLQMNNLKPEDTAVY |
| | extension | YCALGRDYFATDTRQYNYWGLGTLVTVSSGGGGSGGGGSGGGGSGGGGSEVQ |
| | domain that | LVESGGGVVQPGGSLRLSCAASGFTFRSFGMSWVRQAPGKGPEWVSSISGSG |
| | binds albumin) | SDTLYADSVKGRFTISRDNSKNTLYLQMNSLRPEDTALYYCTIGGSLSRSSQ |
| | | GTLVTVSSA |
| 423 | A030900034 | DVQLVESGGGVVQPGGSLRLSCAATGFTLDYYVIGWFRQAPGKEREGVACLS |
| | (ISVD construct | SNGDRINYADSVKGRFTISRDIAKNTVYLQMNSLRPEDTALYHCAAGTSTTV |
| | 2- construct | RDMCGIMYLYDYWAQGTLVTVSSGGGGSGGGSEVQLVESGGGVVQPGGSLRL |
| | comprising PD-1 | SCAATGFTLDYYVIGWFRQAPGKEREGVACLSSNGDRINYADSVKGRFTISR |
| | binding domains | DIAKNTVYLQMNSLRPEDTALYHCAAGTSTTVRDMCGIMYLYDYWAQGTLVT |
| | and IL2Rβ and | VSSGGGGSGGGGSGGGGSGGGGSEVQLVESGGGWVQPGGSLRLSCSPSERLS |
| | IL2Rγ binding | SIDIMGWYRQTPGKEREWVATITGDDTTHYENSVKGRFTISRDTAKNMVYLQ |
| | domains, and a | MNSLEPEDTAVYYCNAGILPREEYWGQGTLVTVSSGGGGSGGGGSGGGGSGG |
| | half-life | GGSEVQLVESGGGLVQAGGSLRLSCAASGRSFSSYYMGWFRQAPGKEREFVA |
| | extension | GINWSGRSTHYADSVKGRFTISRDNAKNTVYLQMNSLKPEDRAAYYCAADSA |
| | domain that | PSTRIGVRSLWDDYDYWGQGTLVTVSSGGGGSGGGGSGGGGSGGGGSEVQLV |
| | binds albumin) | ESGGGVVQPGGSLRLSCAASGFTFRSFGMSWVRQAPGKGPEWVSSISGSGSD |
| | | TLYADSVKGRFTISRDNSKNTLYLQMNSLRPEDTALYYCTIGGSLSRSSQGT |
| | | LVTVSSA |

### Methods

HEK-IL2 cells were plated in 6-well plates the day before and grown to ~60% confluency before treatment. Cells were treated with ISVD constructs (provided in Table 11) in complete growth medium. After incubation at 37° C. for the designated amount of time, cells were washed with PBS, trypsinized and collected by centrifugation at 300 g for 5 min at 4° C. Samples were then tested by western blotting or flow cytometry to quantify protein levels.

### Results

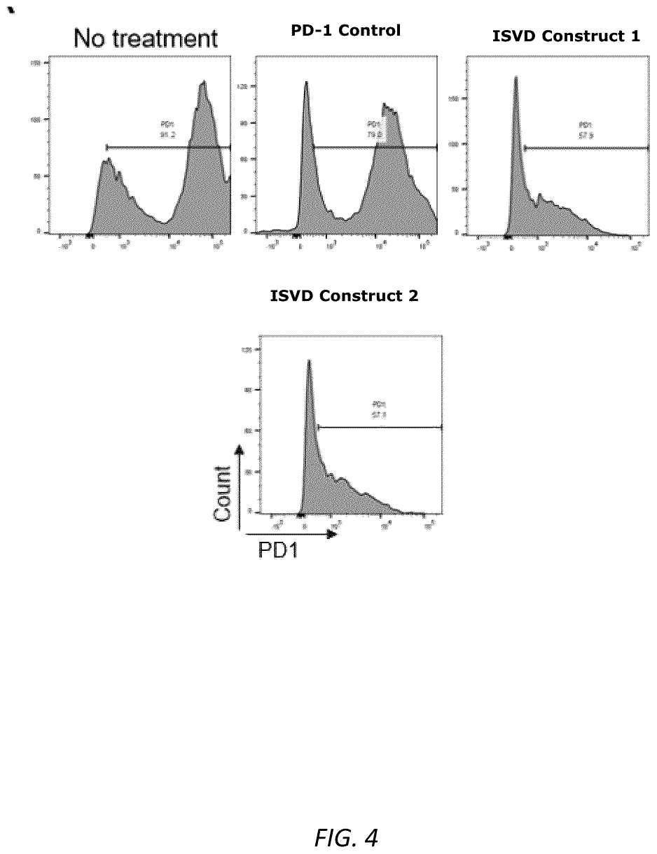

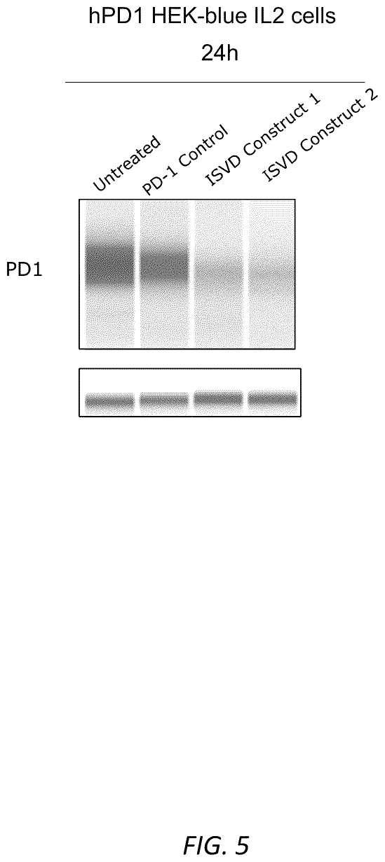

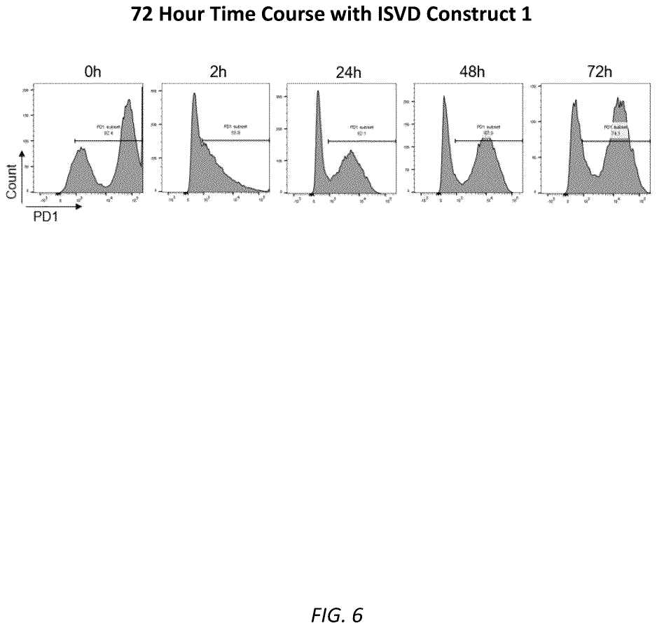

As shown in FIG. 4 and FIG. 5, after incubating the exemplary PD-1/IL-2Rβγ bispecific ISVD constructs in HEK-L2 cells expressing PD-1, the amount of PD-1 after 24-hours were analyzed by western blot and flow cytometry, respectively. PD-1 degradation by the PD-1 control ISVD construct was present but not as significant as the PD-1 degradation displayed by any of the PD-1/IL-2Rβγ ISVD constructs of the disclosure. Table 12 tabulates the half-maximal degradation concentration (DC₅₀) and a maximum degradation efficacy (D_{MAX}) for the constructs tested in FIG. 6.

TABLE 12
PD-1/IL-2Rβγ binding ISVD constructs degradation profile

| Name | DC50 (nM) | D_{MAX} (%) |
|---|---|---|
| A029403748 | 0.13 | 53.78 |
| A030900032 | 0.42 | 75.74 |
| A030900034 | 0.28 | 75.82 |

To further interrogate the timing of PD-1 degradation, ISVD construct 1 was incubated with HEK-L2 cells for a 72-hour time course and the level of PD-1 was analyzed at 0, 2, 24, 48, and 74 hours. As shown in FIG. 6, PD-1 was degraded beginning at 2 hours post incubation and then recovered in expression level after 24 hours.

### Conclusion

The multispecific binding proteins designed herein can bind IL-2Rβγ on IL-2Rβγ positive cells, e.g., HEK-L2 cells, and facilitate the degradation of the target protein (e.g., PD-1).

### Example 6: Multispecific Binding Proteins Induce Target Protein Degradation in Activated T Cells

### Introduction

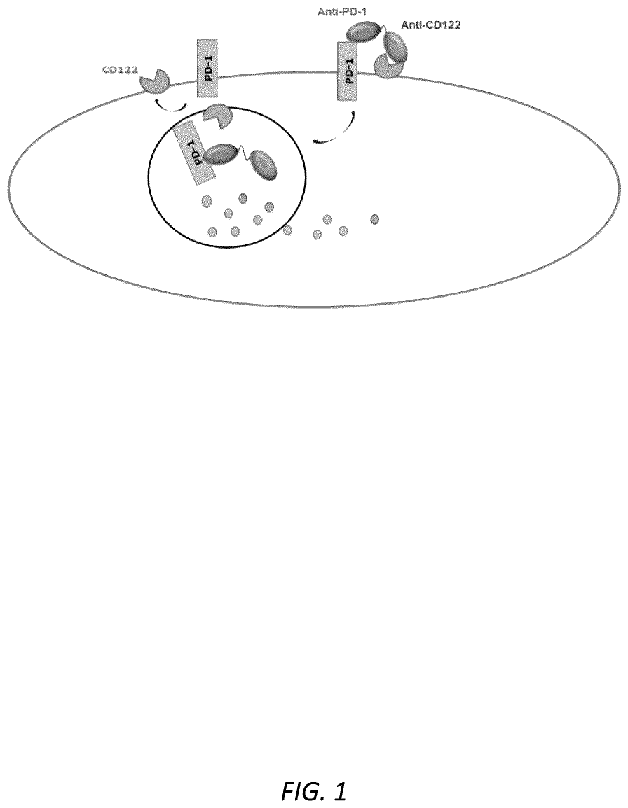

Example 6 tests the hypothesis that exemplary IL-2Rβγ targeting multispecific binding proteins of the disclosure (e.g., ISVD constructs provided in Table 11) can be utilized to shuttle target proteins to IL-2βγ expressing cells for internalization and ultimately for degradation of the target protein (e.g., PD-1) via the lysosomal pathway in immune cells (e.g., activated T cells). Accordingly, the different PD-1/IL-2Rβγ ISVD constructs described in Example 5 were analyzed for the ability to bind PD-1 and subsequently be internalized by IL-2Rβγ as part of the IL2R complex in activated T cells. A schematic of the binding and internalization steps of PD-1 via binding of a PD-1/IL-2Rβγ ISVD construct with trafficking steps of binding and subsequent internalization are shown in FIG. 1.

As shown in FIG. 1, the PD-1/IL-2Rβγ ISVD construct binds to PD-1 and to an IL-2Rβ subunit, an IL-2γ subunit, or both IL-2Rβγ subunits that is part of the human IL-2R complex on IL-2Rβγ positive cells. Subsequently, the PD-1/IL-2Rβγ ISVD construct plus the PD-1 (i.e., the target protein) bound to IL-2R complex is internalized for degradation.

### Methods

T cells isolation and expansion: T cells were isolated from frozen PBMCs using Stemcell T cell isolation Kit. For T cells expansion, 10M isolated cells were resuspended at 0.3-0.5×10⁶/mL in CTS™ AIM V™ SFM medium+50 ng/ml IL-2 in T25 flask. 25 μL/mL of ImmunoCult™ Human CD3/CD28 T Cell Activator was added to the cell suspension and cells were incubated at 37° C. and 5% CO₂ for up to 3 days. A viable cell count was performed every 2-3 days and the cell density adjusted to 5×10⁵ cells/mL whenever the viable cell density reached 2×10⁶ cells/mL. Cells were incubated until the desired cell number was obtained or for up to 12 days.

The degradation assay was similar to what is described in the methods of Example 5.

### Results

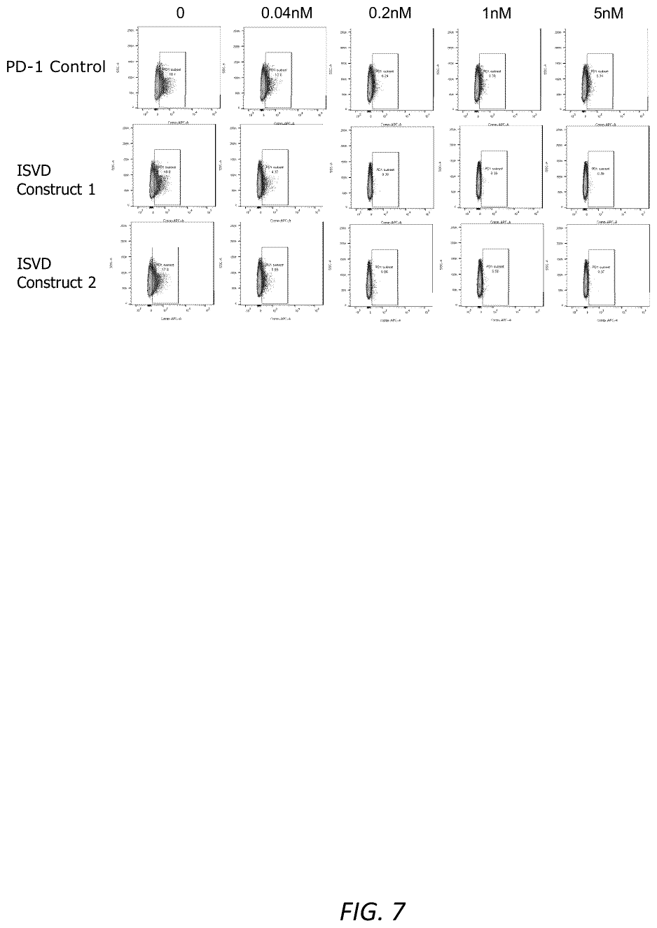

As shown in FIG. 7, PD-1 was internalized after incubation with either the PD-1 ISVD construct control or a PD-1/IL-2Rβγ ISVD construct at low concentrations (e.g., 0.04 nM). At increasing concentrations of the ISVD constructs (0.2 nM-5 nM), the amount of PD-1 signal was diminished. PD-1 signal was completely degraded in T cells after a 24-hour incubation with 5 nM of a PD-1/IL-2Rβγ ISVD construct but not with the PD-1 ISVD construct control. In activated T cells, PD1 degradation was also in a dose-dependent manner with a DC₅₀<0.04 nM.

### Conclusion

The IL-2Rβγ targeting multispecific binding proteins designed herein bind to a target protein, PD-1, and to human IL-2R complex containing IL-2Rβγ. The IL-2R complex containing IL-2Rβγ plus the PD-1/IL-2Rβγ ISVD construct bound to the PD-1, internalized, and degraded in IL-2Rβγ positive immune cells.

1. A method for degrading a target protein, comprising:
contacting an IL-2Rβγ positive cell with a multispecific binding protein, wherein the multispecific binding protein comprises:
a) a first cell surface binding moiety that specifically binds to a CD122 (IL-2Rβ) subunit and/or a CD132 (IL-2Rγ) subunit, on a surface of the IL-2Rβγ positive cell, wherein the first cell surface binding moiety comprises at least one immunoglobulin domain or a fragment thereof; and
b) a second binding moiety that is operatively linked to the first cell surface binding moiety and that specifically binds to the target protein,
wherein specific binding of the multispecific binding protein to the IL-2Rβ subunit and/or the IL-2Rγ subunit facilitates the internalization of the target protein into the IL-2Rβγ positive cell.

2. The method of claim 1, wherein
the IL-2Rβγ positive cell comprises either of a trimer consisting of the IL-2Rβ subunit, the IL-2Rγ subunit, and a CD25 (IL-2Rα) subunit (IL-2Rαβγ) or a dimer consisting of the IL-2Rβ subunit and the IL-2Rγ subunit (IL-2Rβγ);
the target protein traffics to lysosomes for degradation of the target protein;
the IL-2Rβγ positive cell is an immune cell, a white blood cell, or a hematopoietic cell;
the IL-2Rβγ positive cell is a neoplastic cell;
the IL-2Rβγ positive cell is a T-cell or a NK cell;
the first and second binding moiety form a heterodimer, optionally wherein the heterodimer comprises a fragment crystallizable (Fc) domain or variant thereof;
the first cell surface binding moiety binds to an IL-2Rβ subunit epitope, to an IL-2Rγ subunit epitope, or to both an IL-2Rβ subunit epitope and an IL-2Rγ subunit epitope;
the IL-2Rβ subunit epitope and an IL-2Rγ subunit epitope are different epitopes, or the IL-2Rβ subunit epitope and an IL-2Rγ subunit epitope are the same epitopes;
the multispecific binding protein's maximal amount of degradation (DMax) of the target protein is 55, 60, 65, 75, 80, 85, 90, 95, or 100 percent;
the first cell surface binding moiety binds an extracellular domain of the IL-2Rβ subunit, the IL-2Rγ subunit, or both IL-2Rβγ subunits; and/or
the multispecific binding protein exhibits increased degradation of the target protein compared to a reference binding protein, optionally wherein:
the reference binding protein does not comprise the first cell surface binding moiety that specifically binds to the IL-2Rβ subunit, the IL-2Rγ subunit, or both IL-2Rβγ subunits but is otherwise identical to the multispecific binding protein;
the multispecific binding protein exhibits increased degradation of the target protein by at least 2, 3, 4, 5, 10, 20, 30, 40, 50, 60, 70, 80, 90, or 100 percent compared to the reference binding protein; and/or
the multispecific binding protein degrades the target protein at least 2, 3, 4, 5, 10, 24, 48, or 72 hours faster compared to the reference binding protein.

3-19. (canceled)

20. The method of claim 1, wherein:
the first cell surface binding moiety comprises an IL-2Rβ subunit, an IL-2Rγ subunit, or both IL-2Rβγ subunits specific variable domain(s), optionally wherein the IL-2Rβ subunit, the IL-2Rγ subunit, or both IL-2Rβγ subunits specific variable domain(s) is operatively linked to a first Fc domain polypeptide, optionally wherein the first Fc domain polypeptide is an immunoglobulin G (IgG) Fc domain polypeptide;
the second binding moiety that specifically binds to the target protein comprises a target protein specific variable domain, optionally wherein the target protein specific variable domain is operatively linked to a second Fc domain polypeptide, optionally wherein the second Fc domain polypeptide is an immunoglobulin G (IgG) Fc domain polypeptide;
the second binding moiety that specifically binds to the target protein comprises an antibody or an antigen binding fragment thereof, optionally wherein the antibody or an antigen binding fragment thereof comprises an Fc domain or a variant thereof;
the first and second IgG Fc domain polypeptides dimerize to form the multispecific binding protein, optionally wherein the first and second IgG Fc domain polypeptides dimerize by knobs-into-holes interactions, Fab arm exchange (FAE), electrostatic steering interactions, hydrophobic interactions, or any combination thereof, optionally wherein the first IgG Fc domain polypeptide comprises a knob substitution, and the second IgG Fc domain polypeptide comprises a hole substitution or wherein the first IgG Fc domain polypeptide comprises a hole substitution, and the second IgG Fc domain polypeptide comprises a knob substitution, optionally wherein the knob substitution is selected from the group consisting of arginine (R), phenylalanine (F), tyrosine (Y), and tryptophan (W), and/or the hole substitution is selected from the group consisting of alanine (A), asparagine (N), aspartic acid (D), glycine (G), serine(S), threonine (T), and valine (V); and/or
the first cell surface binding moiety and the second binding moiety of the multispecific binding moiety each independently comprises a VH, a Fab, a Fab′, a F(ab′)2, a variable fragment (Fv), a disulfide-stabilized Fv (dsFv), a single-chain Fv (scFv), a diabody, a triabody, an immunoglobulin single variable domain (ISVD), or an AFFIBODY®, optionally wherein:
the scFV is a linear scFV or a tandem scFV;
the multispecific binding protein further comprises a Fc domain or a variant thereof;
the ISVD is a VHH, such as humanized VHH, a camelized VH, a domain antibody (dAb) or a VNAR; and/or
the multispecific binding protein comprises at least three immunoglobulin single variable domains (ISVDs), wherein the at least three ISVDs comprise: a first ISVD that specifically binds to IL-2Rβ, a second ISVD that specifically binds IL-2Rγ, and a third ISVD that specifically binds to the target protein.

21-37. (canceled)

38. The method of claim 1, wherein
the target protein is a membrane-associated target protein, a soluble target protein, or both;
the target protein is expressed on the surface of the same or a different IL-2Rβγ positive cell;
the target protein is expressed on the surface of a neoplastic cell and/or an immune cell;
the target protein is expressed on a T-cell, optionally wherein the T-cell is an activated T cell or a regulatory T (Treg) cell;
the target protein is an immune checkpoint protein, a cancer antigen, and/or an immunomodulatory protein, optionally wherein the immune checkpoint protein is a programmed death 1 (PD1), a programmed death-ligand 1 (PD-L1), or a protein that blocks the interaction between PD1 and PD-L1;
the target protein is selected from the group consisting of: an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein;
the target protein is associated with a disease selected from the group consisting of: a neoplastic disorder, an autoimmune disease, an inflammatory disorder, an infectious disease, and a neurodegenerative disorder;
the multispecific binding protein comprises at least three ISVDs, wherein the at least three ISVDs comprise: a first ISVD that specifically binds to IL-2Rβ, a second ISVD that specifically binds IL-2Rγ, and a third ISVD that blocks PD1/PD-L1 interaction;
the multispecific binding protein exhibits pH-dependent binding to the IL-2Rβ subunit, the IL-2Rγ subunit, the IL-2Rβγ dimer, or the IL-2Rαβγ trimer on the IL-2Rβγ positive cell;
the multispecific binding protein exhibits pH-dependent binding to the target protein;
the multispecific binding protein exhibits reduced binding at acidic pH;
the first cell surface binding moiety of the multispecific binding protein binds to the IL-2Rβ subunit, the IL-2Rγ subunit, the IL-2Rβγ dimer, or the IL-2Rαβγ trimer on the IL-2Rβγ positive cell with an affinity from about 100 pM to about 1 μM; and/or
the second binding moiety of the multispecific binding protein binds to the target protein with an affinity from about 100 pM to about 1 μM.

39-52. (canceled)

53. The method of claim 20,
wherein the first ISVD comprises four framework regions (FR1-FR4) and three complementarity determining regions (CDR1-CDR3), according to Abm numbering, wherein:
(i) the amino acid sequence of CDR1 is at least about 90% identical to an amino acid sequence selected from the group consisting of SEQ ID NOs: 114 and 338; or
(ii) the amino acid sequence of CDR2 is at least about 90% identical an amino acid sequence selected from the group consisting of SEQ ID NOs: 148 and 340; and
(iii) the amino acid sequence of CDR3 is at least about 90% identical to an amino acid sequence selected from the group consisting of SEQ ID NOs: 182 and 342,
optionally wherein
(i) an amino acid sequence of CDR1 comprises an amino acid sequence selected from the group consisting of SEQ ID NOs: 114 and 338;
(ii) an amino acid sequence of CDR2 comprises an amino acid sequence selected from the group consisting of SEQ ID NOs: 148 and 340; and
(iii) an amino acid sequence of CDR3 comprises an amino acid sequence selected from the group consisting of SEQ ID NOs: 182 and 342;
wherein the CDR1, CDR2, and CDR3 amino acid sequences comprise:
(i) the CDR1 corresponding to the amino acid sequence of SEQ ID NO: 114, a CDR2 corresponding to the amino acid sequence of SEQ ID NO: 148, and a CDR3 corresponding to the amino acid sequence of SEQ ID NO: 182; or
(ii) the CDR1 corresponding to the amino acid sequence of SEQ ID NO: 338, a CDR2 corresponding to the amino acid sequence of SEQ ID NO: 340, and a CDR3 corresponding to the amino acid sequence of SEQ ID NO: 342;
wherein the second ISVD comprises four framework regions (FR1-FR4) and three complementarity determining regions (CDR1-CDR3), according to Abm numbering, wherein:
(i) the amino acid sequence of CDR1 is at least about 90% identical to an amino acid sequence selected from the group consisting of SEQ ID NOs: 121 and 331;
(ii) the amino acid sequence of CDR2 is at least about 90% identical to an amino acid sequence selected from the group consisting of SEQ ID NOs: 155 and 334; and
(iii) the amino acid sequence of CDR3 is at least about 90% identical to an amino acid sequence selected from the group consisting of SEQ ID NOs: 189 and 336;
wherein:
(i) an amino acid sequence of CDR1 comprises an amino acid sequence selected from the group consisting of SEQ ID NOs: 121 and 331;
(ii) an amino acid sequence of CDR2 comprises an amino acid sequence selected from the group consisting of SEQ ID NOs: 155 and 334; and
(iii) an amino acid sequence of CDR3 comprises an amino acid sequence selected from the group consisting of SEQ ID NOs: 189 and 336;
wherein the CDR1, CDR2, and CDR3 amino acid sequences comprise:
(i) the CDR1 corresponding to the amino acid sequence of SEQ ID NO: 121, a CDR2 corresponding to the amino acid sequence of SEQ ID NO: 155, and a CDR3 corresponding to the amino acid sequence of SEQ ID NO: 189; or
(ii) the CDR1 corresponding to the amino acid sequence of SEQ ID NO: 331, a CDR2 corresponding to the amino acid sequence of SEQ ID NO: 334, and a CDR3 corresponding to the amino acid sequence of SEQ ID NO: 336;
wherein the first ISVD that specifically binds to IL-2Rβ comprises a sequence that has at least about 85%, about 90%, about 95%, or 100% identity to a sequence selected from the group consisting of SEQ ID Nos. 347 or 360;
wherein the second ISVD that specifically binds IL-2Rγ comprises a sequence that has at least about 85%, about 90%, about 95%, or 100% identity to a sequence selected from the group consisting of SEQ ID Nos. 354 or 359;
wherein the third ISVD that blocks PD1/PD-L1 interaction comprises a sequence that has at least about 85%, about 90%, about 95%, or 100% identity to SEQ ID NO: 361;
wherein the third ISVD that blocks PD1/PD-L1 interaction comprises a sequence that has at least about 85%, about 90%, about 95%, or 100% identity to SEQ ID NO: 362; or
wherein the third ISVD that blocks PD1/PD-L1 interaction comprises a sequence that has at least about 85%, about 90%, about 95%, or 100% identity to SEQ ID NO: 363.

54-63. (canceled)

64. The method of claim 53, wherein
the multispecific binding protein comprises a sequence that has at least about 85%, about 90%, about 95%, or 100% identity to any one of SEQ ID NOs: 364-386;
the multispecific binding protein comprises a human serum albumin binding domain, optionally wherein the human serum albumin domain comprises a sequence selected from the group consisting of SEQ ID Nos. 77-91 and 424-433;
the multispecific binding protein comprises an amino acid sequence that has at least about 85%, about 90%, about 95%, or 100% identity to SEQ ID NO: 422 or 423;
the multispecific binding protein comprises one or more mutations or glycan modifications to modulate Fc mediated effector function; and/or
the multispecific binding protein comprises one or more mutations to modulate serum half-life.

65-69. (canceled)

70. A multispecific binding protein comprising:
a) a first cell surface binding moiety that specifically binds to a CD122 (IL-2Rβ) subunit and/or a CD132 (IL-2Rγ) subunit, on the surface of an IL-2Rβγ positive cell, wherein the first cell surface binding moiety comprises an immunoglobulin domain; and
b) a second binding moiety that is operatively linked to the first cell surface binding moiety and that specifically binds to a target protein,
wherein specific binding of the multispecific binding protein to the IL-2Rβ subunit and/or the IL-2Rγ subunit facilitates internalization of the target protein bound to the multispecific binding protein into the IL-2Rβγ positive cell.

71. The multispecific binding protein of claim 70, wherein
the IL-2Rβγ positive cell comprises either of a trimer consisting of the IL-2Rβ subunit, the IL-2Rγ subunit, and an IL-2Rα subunit (IL-2Rαβγ) or a dimer consisting of the IL-2Rβ subunit and the IL-2Rγ subunit (IL-2Rβγ);
the target protein traffics to lysosomes for degradation of the target protein;
the IL-2Rβγ positive cell is an immune cell, a white blood cell, or a hematopoietic cell;
the IL-2Rβγ positive cell is a neoplastic cell;
the IL-2Rβγ positive cell is a T-cell or a NK cell;
the first and second binding moiety form a heterodimer, optionally wherein the heterodimer comprises a fragment crystallizable (Fc) domain or variant thereof;
the first cell surface binding moiety binds to an IL-2Rβ subunit epitope, an IL-2Rγ subunit epitope, or both an IL-2Rβ epitope and an IL-2Rγ subunit epitope;
the IL-2Rβ epitope and the IL-2Rγ subunit epitope are different epitopes or the IL-2Rβ epitope and the IL-2Rγ subunit epitope are the same epitopes;
the first cell surface binding moiety binds an extracellular domain of the IL-2Rβ subunit, the IL-2Rγ subunit, or both the IL-2Rβγ subunits;
the first cell surface binding moiety comprises an IL-2Rβ subunit, an IL-2Rγ subunit, or both IL-2Rβγ subunits specific variable domain(s), optionally wherein the IL-2Rγ subunit, or both IL-2Rβγ subunits specific variable domain is operatively linked to a first Fc domain polypeptide, optionally wherein the first Fc domain polypeptide is an IgG Fc domain polypeptide;
the second binding moiety that specifically binds to the target protein comprises a target protein specific variable domain, optionally wherein the target protein specific variable domain is operatively linked to a second Fc domain polypeptide, optionally wherein the second Fc domain polypeptide is IgG Fc domain polypeptide;
the second binding moiety that specifically binds to the target protein comprises an antibody or an antigen binding fragment thereof;
the first and second IgG Fc domain polypeptides dimerize to form the multispecific binding protein, optionally wherein the first and second IgG Fc domain polypeptides dimerize by knobs-into-holes interactions, FAE, electrostatic steering interactions, hydrophobic interactions, or any combination thereof, optionally wherein the first IgG Fc domain polypeptide comprises a knob substitution, and the second IgG Fc domain polypeptide comprises a hole substitution or wherein the first IgG Fc domain polypeptide comprises a hole substitution, and the second IgG Fc domain polypeptide comprises a knob substitution, optionally wherein the knob substitution is selected from the group consisting of arginine (R), phenylalanine (F), tyrosine (Y), and tryptophan (W), and/or the hole substitution is selected from the group consisting of alanine (A), asparagine (N), aspartic acid (D), glycine (G), serine(S), threonine (T), and valine (V);
the first cell surface binding moiety and the second binding moieties of the multispecific binding moiety each independently comprises a VH, a Fab, a Fab′, a F(ab′)2, a variable fragment (Fv), a disulfide-stabilized Fv (dsFv), a single-chain Fv (scFv), a diabody, a triabody, an ISVD, or an AFFIBODY®, optionally wherein:
the scFV is a linear scFV or a tandem scFV;
the ISVD is a VHH, humanized VHH, a camelized VH, a domain antibody (dAb), or a VNAR; and/or
the multispecific binding protein comprise at least three ISVDs, wherein the at least three ISVDs comprise: a first ISVD that specifically binds to IL-2Rβ, a second ISVD that specifically binds IL-2Rγ, and a third ISVD that specifically binds to the target protein;
the multispecific binding protein further comprises a Fc domain or a variant thereof;
the target protein is a membrane-associated target protein, a soluble target protein, or both;
the target protein is expressed on the surface of the same or a different IL-2Rβγ positive cell;
the target protein is expressed on the surface of a neoplastic cell and/or an immune cell;
the target protein is expressed on a T-cell, optionally wherein the T-cell is an activated T cell or a regulatory T (Treg) cell;
the target protein is an immune checkpoint protein, a cancer antigen, and/or an immunomodulatory protein, optionally wherein the immune checkpoint protein is PD1, PD-L1, or a protein that blocks the interaction between PD1 and PD-L1;
the target protein is selected from the group consisting of: an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein;
the target protein is associated with a disease selected from the group consisting of: a neoplastic disorder, an autoimmune disease, an inflammatory disorder, an infectious disease, and a neurodegenerative disorder;
the multispecific binding protein comprises at least three ISVDs, wherein the at least three ISVDs comprise: a first ISVD that specifically binds to IL-2Rβ, a second ISVD that specifically binds IL-2Rγ, and a third ISVD that blocks PD1/PD-L1 interaction;
a combination of the first and the second ISVD has an agonistic effect on IL-2Rβγ;
the two subunits of the IL-2Rβ and IL-2Rγ comprise amino acid sequences corresponding to amino acid sequences of SEQ ID NO: 390 and SEQ ID NO: 391, respectively; and/or
the third ISVD of the multispecific binding protein specifically binds to PD-L1.

72-113. (canceled)

114. The multispecific binding protein according claim 71,
wherein the first ISVD comprises four framework regions (FR1-FR4) and three complementarity determining regions (CDR1-CDR3), according to Abm numbering, wherein:
(i) the amino acid sequence of CDR1 is at least about 90% identical to an amino acid sequence selected from the group consisting of SEQ ID NOs: 114 and 338; or
(ii) the amino acid sequence of CDR2 is at least about 90% identical an amino acid sequence selected from the group consisting of SEQ ID NOs: 148 and 340; and
(iii) the amino acid sequence of CDR3 is at least about 90% identical to an amino acid sequence selected from the group consisting of SEQ ID NOs: 182 and 342,
optionally wherein
(i) an amino acid sequence of CDR1 comprises an amino acid sequence selected from the group consisting of SEQ ID NOs: 114 and 338;
(ii) an amino acid sequence of CDR2 comprises an amino acid sequence selected from the group consisting of SEQ ID NOs: 148 and 340; and
(iii) an amino acid sequence of CDR3 comprises an amino acid sequence selected from the group consisting of SEQ ID NOs: 182 and 342;
wherein the CDR1, CDR2, and CDR3 amino acid sequences comprise:
(i) the CDR1 corresponding to the amino acid sequence of SEQ ID NO: 114, a CDR2 corresponding to the amino acid sequence of SEQ ID NO: 148, and a CDR3 corresponding to the amino acid sequence of SEQ ID NO: 182; or
(ii) the CDR1 corresponding to the amino acid sequence of SEQ ID NO: 338, a CDR2 corresponding to the amino acid sequence of SEQ ID NO: 340, and a CDR3 corresponding to the amino acid sequence of SEQ ID NO: 342;
wherein the second ISVD comprises four framework regions (FR1-FR4) and three complementarity determining regions (CDR1-CDR3), according to Abm numbering, wherein:
(i) the amino acid sequence of CDR1 is at least about 90% identical to an amino acid sequence selected from the group consisting of SEQ ID NOs: 121 and 331;
(ii) the amino acid sequence of CDR2 is at least about 90% identical to an amino acid sequence selected from the group consisting of SEQ ID NOs: 155 and 334; and
(iii) the amino acid sequence of CDR3 is at least about 90% identical to an amino acid sequence selected from the group consisting of SEQ ID NOs: 189 and 336;
wherein:
(i) an amino acid sequence of CDR1 comprises an amino acid sequence selected from the group consisting of SEQ ID NOs: 121 and 331;
(ii) an amino acid sequence of CDR2 comprises an amino acid sequence selected from the group consisting of SEQ ID NOs: 155 and 334; and
(iii) an amino acid sequence of CDR3 comprises an amino acid sequence selected from the group consisting of SEQ ID NOs: 189 and 336;
wherein
the CDR1, CDR2, and CDR3 amino acid sequences comprise:
(i) the CDR1 corresponding to the amino acid sequence of SEQ ID NO: 121, a CDR2 corresponding to the amino acid sequence of SEQ ID NO: 155, and a CDR3 corresponding to the amino acid sequence of SEQ ID NO: 189; or
(ii) the CDR1 corresponding to the amino acid sequence of SEQ ID NO: 331, a CDR2 corresponding to the amino acid sequence of SEQ ID NO: 334, and a CDR3 corresponding to the amino acid sequence of SEQ ID NO: 336;
the first ISVD that specifically binds to IL-2Rβ comprises a sequence that has at least about 85%, about 90%, about 95%, or 100% identity to a sequence selected from the group consisting of SEQ ID Nos. 347 or 360;
wherein the second ISVD that specifically binds to IL-2Rγ comprises a sequence that has at least about 85%, about 90%, about 95%, or 100% identity to a sequence selected from the group consisting of SEQ ID Nos. 354 or 359;
wherein the third ISVD that blocks PD1/PD-L1 interaction comprises a sequence that has at least about 85%, about 90%, about 95%, or 100% identity to SEQ ID NO: 361;
wherein the third ISVD that blocks PD1/PD-L1 interaction comprises a sequence that has at least about 85%, about 90%, about 95%, or 100% identity to SEQ ID NO: 362;
wherein the third ISVD that blocks PD1/PD-L1 interaction comprises a sequence that has at least about 85%, about 90%, about 95%, or 100% identity to SEQ ID NO: 363;
wherein the first and second ISVD comprises a sequence that has at least about 85%, about 90%, about 95%, or 100% identity to the first and second ISVD in SEQ ID NO: 364-386;
wherein the multispecific binding protein comprises a human serum albumin binding domain;
wherein the human serum albumin domain comprises a sequence selected from the group consisting of SEQ ID Nos. 77-91 and 424-434;
wherein the multispecific binding protein comprises an amino acid sequence that has at least about 85%, about 90%, about 95%, or 100% identity to SEQ ID NO: 422 or 423;
wherein the relative order of the first, the second, and the third ISVD within the multispecific binding protein from the N-(N) to C-(C) terminal domain are selected from the following orders:
(i): (N)-first ISVD-second ISVD-third ISVD-(C);
(ii): (N)-second ISVD-first ISVD-third ISVD-(C);
(iii): (N)-third ISVD-first ISVD-second ISVD-(C);
(iv): (N)-third ISVD-second ISVD-first ISVD-(C);
(v): (N)-first ISVD-third ISVD-second ISVD-(C); or
(vi): (N)-second ISVD-third ISVD-first ISVD-(C);
wherein said multispecific binding protein further comprises one or more binding moieties operatively linked to the first, second, or third ISVD;
wherein an operative linker is a peptide linker; and/or
wherein the peptide linker is at 90% identical to a peptide linker encoded by an amino acid sequence set forth in SEQ ID NOs: 1-8, 56-58 or 64-76.

115-132. (canceled)

133. The multispecific binding protein according to claim 70, wherein:
the one or more binding moieties increases half-life compared to a reference multispecific binding protein without said one or more binding moieties, optionally wherein a one or more binding moieties facilitates binding to a FcRn, optionally wherein the binding moiety that facilitates binding to a FcRn is selected from a group consisting of: a Fc constant domain, a modified Fc domain, a serum albumin, a serum albumin binding moiety, a serum immunoglobulin, or a variant thereof, optionally wherein the serum albumin is a human serum albumin or the serum immunoglobulin is an IgG, optionally wherein the binding moiety is an ISVD that binds to the human serum albumin, optionally wherein the ISVD that binds to human serum albumin is located proximal to the N- or C-terminal end of the multispecific binding protein;
the multispecific binding protein comprises an amino acid sequence encoding the ISVD that binds to human serum albumin exhibits a sequence identity of more than 90% with an amino acid sequence selected from the group consisting of SEQ ID NOs: 77-91 or 424-433, optionally wherein the amino acid sequence is selected from the group consisting of SEQ ID NOs: 77, 78, 81, 83, or 90;
the multispecific binding protein exhibits pH-dependent binding to IL-2Rβγ on the IL-2Rβγ positive cell;
the multispecific binding protein exhibits pH-dependent binding to the target protein;
the multispecific binding protein exhibits reduced binding at acidic pH;
the first cell surface binding moiety of the multispecific binding protein binds to IL-2Rβγ on the IL-2Rβγ positive cell with an affinity from about 100 pM to about 1 μM;
the second binding moiety of the multispecific binding protein binds to the target protein with an affinity from about 100 pM to about 1 μM;
the multispecific binding protein comprises one or more mutations or glycan modifications to modulate Fc mediated effector function;
the multispecific binding protein comprises one or more mutations to modulate serum half-life; and/or
the multispecific binding protein comprises an amino acid sequence set forth in SEQ ID NO: 422 or 423.

134-148. (canceled)

149. A pharmaceutical composition comprising the multispecific binding protein of claim 70 and a pharmaceutically acceptable carrier or diluent.

150. A composition comprising:
a nucleic acid molecule encoding the multispecific binding protein of claim 70;
a vector comprising the nucleic acid molecule; or
a cell comprising the nucleic acid molecule or the vector.

151-152. (canceled)

153. A method of depleting a target protein comprising administering to a subject an effective amount of the multispecific binding protein of claim 70, optionally wherein:
the multispecific binding protein is internalized by the IL-2Rβγ positive cell, optionally wherein an amount of multispecific binding protein internalized by the IL-2Rβγ positive cell is greater than an amount of a reference binding protein that does not comprise the first cell surface binding moiety; and/or
the target protein is selectively depleted from a target tissue or circulation of the subject, optionally wherein administering the multispecific binding protein results in at least about 10%, 20, 30%, 40%, 50%, 75%, or 90% depletion of the target protein from the target tissue or circulation of the subject.

154-157. (canceled)

158. A method of treating a disease comprising administering an effective amount of a multispecific binding protein of claim 70 to a subject, optionally wherein the disease is selected from a group consisting of: a neoplastic disorder, an autoimmune disease, an inflammatory disorder, an infectious disease, or a neurodegenerative disorder, optionally wherein the composition according to claim 70 is used in a treatment of a checkpoint refractory solid tumor.

159-160. (canceled)

161. A method for producing a multispecific binding protein according to claim 70, said method at least comprising the steps of:
a) expressing, in a suitable host cell or host organism or in another suitable expression system, a nucleic acid expressing the multispecific binding protein;
optionally followed by:
b) isolating and/or purifying the multispecific binding protein.

162. An immunoglobulin single variable domain (ISVD) specifically binding human IL2Rgamma, that essentially consists of 4 framework regions (FR1 to FR4, respectively) and 3 complementarity determining regions (CDR1 to CDR3, respectively), in which:
CDR1 (AbM numbering) has an amino acid sequence selected from:
a) the amino acid sequence of SEQ ID NO: 121;
b) amino acid sequences that have at least 80% amino acid identity with the amino acid sequence of SEQ ID NO: 121; and
c) amino acid sequences that have 3, 2, or 1 amino acid difference with the amino acid sequences of SEQ ID NO: 121;
and
CDR2 (AbM numbering) has an amino acid sequence selected from:
d) the amino acid sequence of SEQ ID NO: 155;
e) amino acid sequences that have at least 80% amino acid identity with the amino acid sequence of SEQ ID NO: 155;
f) amino acid sequences that have 3, 2, or 1 amino acid difference with the amino acid sequence of SEQ ID NO: 155;
and:
CDR3 (AbM numbering) has an amino acid sequence selected from:
g) the amino acid sequence of SEQ ID NO: 189;
h) amino acid sequences that have at least 80% amino acid identity with the amino acid sequence of SEQ ID NO: 189;
i) amino acid sequences that have 3, 2, or 1 amino acid difference with the amino acid sequence of SEQ ID NO: 189; optionally
wherein the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 354; and/or
wherein
CDR1 consists of the amino acid sequence of SEQ ID NO: 121;
CDR2 consists of the amino acid sequences of SEQ ID NO: 155; and
CDR3 consists of the amino acid sequence of SEQ ID NO: 189; or
an ISVD specifically binding human IL2Rbeta, that essentially consists of 4 framework regions (FR1 to FR4, respectively) and 3 complementarity determining regions (CDR1 to CDR3, respectively), in which:
CDR1 (AbM numbering) has an amino acid sequence selected from:
a) the amino acid sequence of SEQ ID NO: 114;
b) amino acid sequences that have at least 80% amino acid identity with the amino acid sequence of SEQ ID NO: 114; and
c) amino acid sequences that have 3, 2, or 1 amino acid difference with the amino acid sequences of SEQ ID NO: 114;
and
CDR2 (AbM numbering) has an amino acid sequence selected from:
d) the amino acid sequence of SEQ ID NO: 148;
e) amino acid sequences that have at least 80% amino acid identity with the amino acid sequence of SEQ ID NO: 148;
f) amino acid sequences that have 3, 2, or 1 amino acid difference with the amino acid sequence of SEQ ID NO: 148;
and
CDR3 (AbM numbering) has an amino acid sequence selected from:
g) the amino acid sequence of SEQ ID NO: 182;
h) amino acid sequences that have at least 80% amino acid identity with the amino acid sequence of SEQ ID NO: 182;
i) amino acid sequences that have 3, 2, or 1 amino acid difference with the amino acid sequence of SEQ ID NO: 182; optionally
wherein the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NOs: 347; and/or
in which
CDR1 consists of the amino acid sequence of SEQ ID NO: 114;
CDR2 consists of the amino acid sequences of SEQ ID NO: 148; and
CDR3 consists of the amino acid sequence of SEQ ID NO: 182; or
an ISVD specifically binding human IL2Rgamma, that essentially consists of 4 framework regions (FR1 to FR4, respectively) and 3 complementarity determining regions (CDR1 to CDR3, respectively), in which:
CDR1 (AbM numbering) has an amino acid sequence selected from:
a) the amino acid sequence of SEQ ID NO: 331;
b) amino acid sequences that have at least 80% amino acid identity with the amino acid sequence of SEQ ID NO: 331; and
c) amino acid sequences that have 3, 2, or 1 amino acid difference with the amino acid sequences of SEQ ID NO: 331;
and
CDR2 (AbM numbering) has an amino acid sequence selected from:
d) the amino acid sequence of SEQ ID NO: 334;
e) amino acid sequences that have at least 80% amino acid identity with the amino acid sequence of SEQ ID NO: 334;
f) amino acid sequences that have 3, 2, or 1 amino acid difference with the amino acid sequence of SEQ ID NO: 334;
and
CDR3 (AbM numbering) has an amino acid sequence selected from:
g) the amino acid sequence of SEQ ID NO: 336;
h) amino acid sequences that have at least 80% amino acid identity with the amino acid sequence of SEQ ID NO: 336;
i) amino acid sequences that have 3, 2, or 1 amino acid difference with the amino acid sequence of SEQ ID NO: 336; optionally
wherein the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 359; and/or
wherein
CDR1 consists of the amino acid sequence of SEQ ID NO: 331;
CDR2 consists of the amino acid sequences of SEQ ID NO: 334; and
CDR3 consists of the amino acid sequence of SEQ ID NO: 336; or
an ISVD specifically binding human IL2Rbeta, that essentially consists of 4 framework regions (FR1 to FR4, respectively) and 3 complementarity determining regions (CDR1 to CDR3, respectively), in which:
CDR1 (AbM numbering) has an amino acid sequence selected from:
a) the amino acid sequence of SEQ ID NO: 338;
b) amino acid sequences that have at least 80% amino acid identity with the amino acid sequence of SEQ ID NO: 338; and
c) amino acid sequences that have 3, 2, or 1 amino acid difference with the amino acid sequences of SEQ ID NO: 338;
and
CDR2 (AbM numbering) has an amino acid sequence selected from:
d) the amino acid sequence of SEQ ID NO: 340;
e) amino acid sequences that have at least 80% amino acid identity with the amino acid sequence of SEQ ID NO: 340
f) amino acid sequences that have 3, 2, or 1 amino acid difference with the amino acid sequence of SEQ ID NO: 340;
and
CDR3 (AbM numbering) has an amino acid sequence selected from:
g) the amino acid sequence of SEQ ID NO: 342;
h) amino acid sequences that have at least 80% amino acid identity with the amino acid sequence of SEQ ID NO: 342;
i) amino acid sequences that have 3, 2, or 1 amino acid difference with the amino acid sequence of SEQ ID NO: 342; optionally
wherein the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 360; and/or
wherein
CDR1 consists of the amino acid sequence of SEQ ID NO: 338;
CDR2 consists of the amino acid sequences of SEQ ID NO: 340; and
CDR3 consists of the amino acid sequence of SEQ ID NO: 342; or
an ISVD specifically binding human IL2Rgamma, that essentially consists of 4 framework regions (FR1 to FR4, respectively) and 3 complementarity determining regions (CDR1 to CDR3, respectively), in which:
CDR1 (Kabat numbering) has an amino acid sequence selected from:
a) the amino acid sequence of SEQ ID NO:443;
b) amino acid sequences that have at least 80% amino acid identity with the amino acid sequence of SEQ ID NO: 443;
c) amino acid sequences that have 3, 2, or 1 amino acid difference with the amino acid sequences of SEQ ID NO: 443;
and
CDR2 (Kabat numbering) has an amino acid sequence selected from:
d) the amino acid sequence of SEQ ID NO: 451;
e) amino acid sequences that have at least 80% amino acid identity with the amino acid sequence of SEQ ID NO: 451;
f) amino acid sequences that have 3, 2, or 1 amino acid difference with the amino acid sequence of SEQ ID NO: 451;
and
CDR3 (Kabat numbering) has an amino acid sequence selected from:
g) the amino acid sequence of SEQ ID NO: 459;
h) amino acid sequences that have at least 80% amino acid identity with the amino acid sequence of SEQ ID NO: 459;
i) amino acid sequences that have 3, 2, or 1 amino acid difference with the amino acid sequence of SEQ ID NO: 459; optionally
wherein the amino acid sequences of the CDRs (Kabat numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 354; and/or
wherein
CDR1 consists of the amino acid sequence of SEQ ID NO: 443;
CDR2 consists of the amino acid sequences of SEQ ID NO: 451; and
CDR3 consists of the amino acid sequence of SEQ ID NO: 459; or
an ISVD specifically binding human IL2Rbeta, that essentially consists of 4 framework regions (FR1 to FR4, respectively) and 3 complementarity determining regions (CDR1 to CDR3, respectively), in which:
CDR1 (Kabat numbering) has an amino acid sequence selected from:
a) the amino acid sequence of SEQ ID NO: 444;
b) amino acid sequences that have at least 80% amino acid identity with the amino acid sequence of SEQ ID NO: 444;
c) amino acid sequences that have 3, 2, or 1 amino acid difference with the amino acid sequences of SEQ ID NO: 444;
and
CDR2 (Kabat numbering) has an amino acid sequence selected from:
d) the amino acid sequence of SEQ ID NO: 452;
e) amino acid sequences that have at least 80% amino acid identity with the amino acid sequence of SEQ ID NO: 452;
f) amino acid sequences that have 3, 2, or 1 amino acid difference with the amino acid sequence of SEQ ID NO: 452;
and
CDR3 (Kabat numbering) has an amino acid sequence selected from:
g) the amino acid sequence of SEQ ID NO: 460;
h) amino acid sequences that have at least 80% amino acid identity with the amino acid sequence of SEQ ID NO: 460;
i) amino acid sequences that have 3, 2, or 1 amino acid difference with the amino acid sequence of SEQ ID NO: 460; optionally
wherein the amino acid sequences of the CDRs (Kabat numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 347; and/or
wherein
CDR1 consists of the amino acid sequence of SEQ ID NO: 444;
CDR2 consists of the amino acid sequences of SEQ ID NO: 452; and
CDR3 consists of the amino acid sequence of SEQ ID NO: 460; or
an ISVD specifically binding human IL2Rgamma, that essentially consists of 4 framework regions (FR1 to FR4, respectively) and 3 complementarity determining regions (CDR1 to CDR3, respectively), in which:
CDR1 (Kabat numbering) has an amino acid sequence selected from:
a) the amino acid sequence of SEQ ID NO: 445;
b) amino acid sequences that have at least 80% amino acid identity with the amino acid sequence of SEQ ID NO: 445;
c) amino acid sequences that have 3, 2, or 1 amino acid difference with the amino acid sequences of SEQ ID NO: 445;
and
CDR2 (Kabat numbering) has an amino acid sequence selected from:
d) the amino acid sequence of SEQ ID NO: 453;
e) amino acid sequences that have at least 80% amino acid identity with the amino acid sequence of SEQ ID NO: 453;
f) amino acid sequences that have 3, 2, or 1 amino acid difference with the amino acid sequence of SEQ ID NO: 453;
and
CDR3 (Kabat numbering) has an amino acid sequence selected from:
g) the amino acid sequence of SEQ ID NO: 461;
h) amino acid sequences that have at least 80% amino acid identity with the amino acid sequence of SEQ ID NO: 461;
i) amino acid sequences that have 3, 2, or 1 amino acid difference with the amino acid sequence of SEQ ID NO: 461; optionally
wherein the amino acid sequences of the CDRs (Kabat numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 359; and/or
wherein
CDR1 consists of the amino acid sequence of SEQ ID NO: 445;
CDR2 consists of the amino acid sequences of SEQ ID NO: 453; and
CDR3 consists of the amino acid sequence of SEQ ID NO: 461; or
an ISVD specifically binding human IL2Rbeta, that essentially consists of 4 framework regions (FR1 to FR4, respectively) and 3 complementarity determining regions (CDR1 to CDR3, respectively), in which:
CDR1 (Kabat numbering) has an amino acid sequence selected from:
a) the amino acid sequence of SEQ ID NO: 446;
b) amino acid sequences that have at least 80% amino acid identity with the amino acid sequence of SEQ ID NO: 446;
c) amino acid sequences that have 3, 2, or 1 amino acid difference with the amino acid sequences of SEQ ID NO: 446;
and
CDR2 (Kabat numbering) has an amino acid sequence selected from:
d) the amino acid sequence of SEQ ID NO: 454;
e) amino acid sequences that have at least 80% amino acid identity with the amino acid sequence of SEQ ID NO: 454;
f) amino acid sequences that have 3, 2, or 1 amino acid difference with the amino acid sequence of SEQ ID NO: 454;
and
CDR3 (Kabat numbering) has an amino acid sequence selected from:
g) the amino acid sequence of SEQ ID NO: 462;
h) amino acid sequences that have at least 80% amino acid identity with the amino acid sequence of SEQ ID NO: 462;
i) amino acid sequences that have 3, 2, or 1 amino acid difference with the amino acid sequence of SEQ ID NO: 462; optionally
wherein the amino acid sequences of the CDRs (Kabat numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NO: 360; and/or
wherein
CDR1 consists of the amino acid sequence of SEQ ID NO: 446;
CDR2 consists of the amino acid sequences of SEQ ID NO: 454; and
CDR3 consists of the amino acid sequence of SEQ ID NO: 462.

163-185. (canceled)

186. The ISVD according to claim 162, in wherein the amino acid sequences of the CDRs (Chothia definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360;
wherein the amino acid sequences of the CDRs (IMGT definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 354, 347, 359, and 360;
wherein the amino acid sequence
i) has 80% amino acid sequence identity with one of the amino acid sequences of SEQ ID NOs: 354, 347, 359, and 360, in which for the purposes of determining the degree of amino acid identity, the amino acid residues that form the CDR sequences are disregarded;
and in which:
ii) preferably one or more of the amino acid residues at positions 11, 37, 44, 45, 47, 83, 84, 103, 104 and 108 according to the Kabat numbering are chosen from the Hallmark residues mentioned in Table 1;
wherein the ISVD consists essentially of a heavy chain variable domain sequence that is derived from a conventional four-chain antibody or that consists essentially of a heavy chain variable domain sequence that is derived from heavy chain antibody;
wherein the ISVD consists essentially of a VHH, a humanized VHH, a camelized VH, a domain antibody, a single domain antibody, or a dAb;
wherein the amino acid sequence is chosen from the group consisting of SEQ ID NOs: 354, 347, 359, and 360 or from the group of amino acid sequences that have more than 80%, preferably more than 90%, more preferably more than 95%, such as 99% or more amino acid sequence identity with at least one amino acid sequence selected from SEQ ID NOs: 354, 347, 359, and 360,
wherein the ISVD specifically binds to human and cyno IL2R-gamma and/or IL2R-beta and does not bind to IL21R or other members of the IL2R family; and/or
wherein the ISVD antagonizes an activity of IL2RBeta and/or IL2Rgamma.

187-193. (canceled)

194. A polypeptide or construct that comprises or essentially consists of one or more ISVDs according to claim 162, and optionally
wherein the polypeptide or construct further comprises one or more other groups, residues, moieties or binding units, optionally linked via one or more linkers;
wherein the one or more other groups, residues, moieties or binding units are amino acid sequences;
wherein the one or more linkers are one or more amino acid sequences;
wherein the one or more other groups, residues, moieties or binding units are immunoglobulin sequences;
wherein the one or more other groups, residues, moieties or binding units are ISVDs;
wherein the one or more other groups, residues, moieties or binding units are chosen from the group consisting of VHHs, humanized VHHs, camelized VHs, domain antibodies, single domain antibodies and dAbs;
wherein the polypeptide or construct is a multivalent construct;
wherein the polypeptide or construct is a multispecific construct;
wherein the one or more other groups, residues, moieties or binding units provide the polypeptide or construct with increased half-life, compared to the ISVD without the one or more other groups, residues, moieties or binding units;
wherein the one or more other groups, residues, moieties or binding units that provide the polypeptide or construct with increased half-life is chosen from the group consisting of a polyethylene glycol molecule (PEG), serum proteins or fragments thereof, binding units that specifically bind to serum proteins, an Fc portion, and small proteins or peptides that specifically bind to serum proteins;
wherein the one or more other groups, residues, moieties or binding units that provide the polypeptide or construct with increased half-life is chosen from the group consisting of human serum albumin or fragments thereof:
wherein the one or more other groups, residues, moieties or binding units that provides the polypeptide or construct with increased half-life are chosen from the group consisting of binding units that specifically bind to serum albumin (such as human serum albumin) or a serum immunoglobulin (such as IgG);
wherein the one or more other groups, residues, moieties or binding units that provides the polypeptide or construct with increased half-life are chosen from the group consisting of VHHs, humanized VHHs, camelized VHs, domain antibodies, single domain antibodies, or dAbs that specifically bind to serum albumin (such as human serum albumin) or a serum immunoglobulin (such as IgG);
wherein the one or more other groups, residues, moieties or binding units that provides the polypeptide or construct with increased half-life is an ISVD that specifically binds human serum albumin;
wherein the ISVD that specifically binds human serum albumin essentially consists of 4 framework regions (FR1 to FR4, respectively) and 3 complementarity determining regions (CDR1 to CDR3 respectively), in which:
CDR1 (AbM numbering) has an amino acid sequence selected from:
a) the amino acid sequence of SEQ ID NO: GFTFRSFGMS (SEQ ID NO: 467);
b) amino acid sequences that have at least 80% amino acid identity with the amino acid sequence of SEQ ID NO: GFTFRSFGMS (SEQ ID NO: 467); and
c) amino acid sequences that have 3, 2, or 1 amino acid difference with the amino acid sequences of SEQ ID NO: GFTFRSFGMS (SEQ ID NO: 467);
and
CDR2 (AbM numbering) has an amino acid sequence selected from:
d) the amino acid sequence of SEQ ID NO: SISGSGSDTL (SEQ ID NO: 468);
e) amino acid sequences that have at least 80% amino acid identity with the amino acid sequence of SEQ ID NO: SISGSGSDTL (SEQ ID NO: 468);
f) amino acid sequences that have 3, 2, or 1 amino acid difference with the amino acid sequence of SEQ ID NO: SISGSGSDTL (SEQ ID NO: 468);
and
CDR3 (AbM numbering) has an amino acid sequence selected from:
g) the amino acid sequence of SEQ ID NOs: GGSLSRS (SEQ ID NO: 469);
h) amino acid sequences that have at least 80% amino acid identity with the amino acid sequence of SEQ ID NO: GGSLSRS (SEQ ID NO: 469);
i) amino acid sequences that have 3, 2, or 1 amino acid difference with the amino acid sequence of SEQ ID NO: GGSLSRS (SEQ ID NO: 469);
wherein the CDR1 consists of the amino acid sequence of SEQ ID NO: GFTFRSFGMS (SEQ ID NO: 467), CDR2 consists of the amino acid sequence of SEQ ID NO: SISGSGSDTL (SEQ ID NO: 468), and CDR3 consists of the amino acid sequence of SEQ ID NO: GGSLSRS (SEQ ID NO: 469);
wherein the ISVD that specifically binds human serum albumin is selected from the group consisting of ALB8 (SEQ ID NO: 77), ALB23 (SEQ ID NO: 78), and ALB23002 (SEQ ID NO: 90);
wherein the linker is chosen from the group consisting of SEQ ID NOs: 1-8, 56-58, 64-73;
wherein the polypeptide or construct further comprises a C-terminal extension; and/or
wherein the C-terminal extension is a C-terminal extension (X)ₙ, in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).

195-213. (canceled)

214. A nucleic acid that encodes an ISVD according to claim 162, optionally wherein the nucleic acid is in the form of a genetic construct, and/or wherein a non-human host or host cell that expresses, or that under suitable circumstances is capable of expressing, an ISVD according to claim 162.

215-216. (canceled)

217. A method for producing an ISVD according to claim 162, at least comprising the steps of:
a) expressing, in a suitable (non-human) host cell or host organism or in another suitable expression system, a nucleic acid encoding the ISVD;
optionally followed by:
b) isolating and/or purifying the ISVD; or
a method for producing an ISVD according to claim 162, said method at least comprising the steps of:
a) cultivating and/or maintaining a non-human host or host cell under conditions that are such that said non-human host or host cell expresses and/or produces at least one ISVD according to claim 162;
optionally followed by:
b) isolating and/or purifying the ISVD according to claim 162.

218. (canceled)

219. A composition comprising at least one ISVD according to claim 162, optionally wherein
the composition is a pharmaceutical composition, and/or wherein the pharmaceutical composition, further comprises at least one pharmaceutically acceptable carrier, diluent or excipient and/or adjuvant, and
that optionally comprises one or more further pharmaceutically active polypeptides and/or compounds.

220-225. (canceled)

226. A method for the diagnosis, prevention and/or treatment of at least one disease and/or disorder, comprising the administration, to a subject in need thereof, of a pharmaceutically active amount of an ISVD according to claim 219, optionally wherein
the at least one disease and/or disorder is cancer or associated with IL2R beta and/or IL2R-gamma, with its biological or pharmacological activity, and/or with the biological pathways or signalling in which IL2R beta and/or IL2R-gamma is involved.

227-228. (canceled)
